(19) United States
(12) Patent Application Publication
(10) Pub. No.: US 2020/0056206 A1
(43) Pub. Date: Feb. 20, 2020

(54) CRISPR-BASED TREATMENT OF FRIEDREICH ATAXIA

(71) Applicant: UNIVERSITÉ LAVAL, QUÉBEC (CA)

(72) Inventors: JACQUES P. TREMBLAY, QUÉBEC (CA); DOMINIQUE L. OUELLET, BELMONT, MA (US)

(21) Appl. No.: 16/464,870

(22) PCT Filed: Dec. 1, 2017
(86) PCT No.: PCT/CA2017/051448

§ 371 Date: May 29, 2019

Related U.S. Application Data
(60) Provisional application No. 62/428,809, filed on Dec. 1, 2016.

(51) Int. Cl. C12N 15/90; A61K 31/7105; A61P 25/14; A61P 25/28; C07K 14/47; C12N 15/113; C12N 9/22; A61K 48/00

(52) U.S. Cl. CPC C12N 15/907; A61K 31/7105; A61P 25/14; A61P 25/28; C07K 14/47; C12N 15/113; C12N 2750/14143; A61K 48/0016; A61K 48/005; A01K 2217/075; A01K 2227/105; A01K 2267/0306; C12N 2310/20; C12N 9/22

(57) ABSTRACT

Methods of modifying a frataxin gene are disclosed, comprising removing some or all of endogenous GAA trinucleotide repeats within the frataxin gene, e.g., within an intron (e.g., intron 1) of the frataxin gene. The removal may be effected using a CRISPR/CAS nuclease system. Such modification may be used to increase frataxin expression in the cell, and also to treat a subject suffering from Friedreich ataxia. Reagents, kits and uses of the method are also disclosed, for example to modify a frataxin gene and to treat a subject suffering from Friedreich ataxia.

### CROSS REFERENCE TO RELATED APPLICATIONS

This application is a National Stage Application of PCT Application No. PCT/CA2017/051448 filed on Dec. 1, 2017 and published in English under PCT Article 21(2), which claims the benefit of US provisional application Ser. No. 62/428,809, filed on Dec. 1, 2016. All documents above are incorporated herein by reference in their entirety.

### FIELD OF THE INVENTION

The present invention relates to the targeted modification of an endogenous mutated frataxin (FXN) gene to restore or increase FXN expression in mutated cells, such as cells of subjects suffering from Friedreich ataxia (FRDA). More specifically, the present invention is concerned with removing abnormal GAA repeats in intron 1 of a mutated frataxin gene by targeting polynucleotide sequences close to the endogenous GM repeat extension.

### REFERENCE TO SEQUENCE LISTING

Pursuant to 37 C.F.R. 1.821(c), a sequence listing is submitted herewith as an ASCII compliant text file named “G11229-397-SL-ST25-v2.txt”, created on May 28, 2019 and having a size of about 262 Kbytes 264 KB, which is incorporated herein by reference in its entirety.

### BACKGROUND OF THE INVENTION

Friedreich ataxia (FRDA) is an inherited autosomal recessive neurodegenerative disease with symptoms appearing usually within the second decade of life. The phenotypic expression is characterized by a progressive ataxia with uncoordinated movements, weakened muscle strength and balance problems (1-5). Some FRDA patients also have systemic impairments including, but not restricted to, cardiomyopathy, diabetes mellitus and scoliosis (6). Early death in FRDA subjects results from cardiomyopathy or associated arrhythmias (3).

The FXN protein is essential for adequate mitochondrial functioning. It is involved in the incorporation of iron into heme and iron-sulfur clusters (14). When FXN is deficient, iron is misdirected and this leads to oxidative stress. In FRDA, reduced levels of frataxin (FXN) protein in the mitochondria cause oxidative damages and iron deficiencies at the cellular level (7). Neurons and cardiomyocytes are particularly sensitive to this stress (7) although all tissues are affected to some extent. The reduced FXN expression has been linked to a GAA triplet expansion within intron 1 of the somatic and germline FXN gene (8). In FRDA patients, the GAA repeat expansion generally consists of more than 70 GM repeats with some individuals having a large expansion of up to 1700 GAA repeats. Most affected individuals have 600 to 900 GAA triplets, whereas unaffected individuals commonly have about 40-64 repeats in the FXN gene (9). The number of GAA repeats correlates with the severity of the disease and is inversely proportional with the age of onset. The effect of the repeat expansion is to significantly decrease expression of the essential and ubiquitous FXN mitochondrial protein. Asymptomatic carriers express about 50% of FXN compared to unaffected individuals.

FXN gene silencing is taught to occur via at least two, non-mutually exclusive mechanisms of action: (i) Repeat expansions adopt abnormal B DNA structures (triplexes or “sticky” DNA) or DNA:RNA hybrid structures (known as R-loops) which impede RNA polymerase activity and thus reduce gene transcription of the FXN gene; and/or (ii) Repeat expansions can produce heterochromatin-mediated gene silencing effects through various epigenetic mechanisms (such as DNA methylation, histone modification, chromatin remodelling, and noncoding RNAs), resulting in heritable changes in gene expression that do not involve changes in DNA sequence. A reduced level of FXN has been shown to lead to changes in the expression of over 185 different genes (12, 13).

Altered DNA structure (triplexes, sticky DNA and/or R-Loops) of the FXN gene in FRDA cells: (a) creates a physical blockage on RNA polymerase II (RNAPII) transcription machinery, affecting both transcription initiation and elongation. Formation of sticky DNA is thought to impair transcription by creating a physical barrier effect on transcription by making it more difficult for the elongating RNAPII complex to unwind the DNA template and move forward (53, 55, 56); (b) induces FXN antisense transcription. R-Loops increase RNAPII pausing and induce antisense transcription. Increased level of a FAST-1 antisense corresponding to the antisense of the FXN transcript was detected in FRDA cells. Such antisense is thought to contribute to the negative regulation of FXN expression (57); and (c) promotes heterochromatin formation, leading to gene silencing. Recruitment of transcriptional activators and initiation of transcription at the promoter is affected by the spreading of a heterochromatin-like environment. Indeed, evidence of heterochromatin formation was found in the vicinity (including the promoter region) of the expanded GAA triplets in FRDA patients (57) (e.g., increase levels of histone methylation, hydroxymethylation and hypoacetylation). Also, administration of histone deacetylase (HDAC) inhibitors was shown to increase FXN expression in cells of Friedreich Ataxia patients. In mouse experiments, the expanded GAA triplet repeat sequence was found to be a source of position effect and to silence genes which were adjacent to the repeat sequence (through heterochromatin spreading) (57). Furthermore, the unusual/altered DNA conformation of the mutated FXN gene has been shown to be recognized by the cell mismatch-repair system. Evidence suggests that recruitment of the mismatch-repair system (and/or inducement of FXN antisense transcription) triggers the recruitment of chromatin modifiers leading to heterochromatin formation and spreading. Studies have shown that cells from FRDA patients are depleted in chromatic insulator protein CTCF, which is associated with increased heterochromatin formation at the transcription start site of the FXN gene. CTCF acts by promoting higher order chromatin organization known to regulate gene expression via the creation boundaries in chromatin. Depletion of CTCF in FRDA subjects is thought to promote heterochromatin spreading and contribute to gene silencing (57).

Thus, the mutant FXN gene in cells from FRDA subjects suffers from deficient transcriptional initiation and elongation, and also suffers from FXN antisense transcription and heterochromatin formation, as the mechanisms of action of its overall defective transcription. See Sandi et al., 2014 (55), Sandi et al., 2013 (54), Kumari et al., 2011 (53), De Biase et al, 2009 (57), Pandolfo et al, 2012 (7) and Yandim et al, 2013 (56). The unusual compact heterochormatin structure of the FXN gene in FRDA complicates targeting of molecular complex (e.g., gRNA/Cas9 complex) on the gene and render their effects uncertain and/or unpredictable.

Several strategies have been developed for treating Friedreich ataxia. These fall generally into the following 5 categories: 1) use of antioxidants to reduce the oxidative stress caused by iron accumulation in the mitochondria; 2) use of iron chelators to remove iron from the mitochondria; 3) use of Histone Deacetylase Inhibitors (HDACIs) to prevent DNA condensation and permit higher expression of FXN; 4) use of molecules such as cisplatin, 3-nitroproprionnic acid (3-NP), Pentamidine or erythropoietin (EPO) to boost FXN expression; and 5) gene therapy. Antioxidants and iron chelators are currently under investigation in clinical trials (7). However, limited success has been reported thus far for these strategies, which generally involve continued treatment throughout the life of the patient. Thus, there remains a need for new approaches for treating or preventing Friedreich ataxia and symptoms associated with FRDA.

The present description refers to a number of documents, the content of which is herein incorporated by reference in their entirety.

### SUMMARY OF THE INVENTION

Recently, gene replacement or gene editing has made an important comeback with the development of the CRISPR-based system derived from bacteria. In bacteria and archaea, the CRISPR RNA (crRNA) and transactivating CRISPR RNA (tracrRNA) form a complex, which acts as the homing device for directing a nuclease (Cas9) to invading foreign genetic materials. CRISPR technology uses a nuclease (e.g., Cas9) and a guide RNA (gRNA) containing a variable sequence of about 20 nucleotides (crRNA), complementary to the targeted DNA sequence, to induce breaks (doubled stranded or single stranded breaks (DSBs or SSBs)) in DNA (15-18). A constant RNA sequence (e.g., of about 42 nucleotides or more (tracrRNA)) may be linked to the variable region of the guide RNA or be provided as a separate entity.

Introduction of DSBs can knockout a specific gene or allow modifying it by Homology Directed Repair (HDR). CRISPR-Cas9-induced DNA cleavage followed by Non-Homologous End Joining (NHEJ) repair has been used to generate loss-of-function alleles in protein-coding genes or to delete a very large DNA fragment (20, 21). The off-target mutation rate has also been significantly reduced by modifying the Cas9 nuclease (22, 23). Although not all possible gRNAs targeting specific target sequences are found to be equally useful and, although the identification of useful target region/sequences often still remains unpredictable, the CRISPR-Cas system is nevertheless an exciting tool for the development of therapies involving gene editing.

The present invention thus relates to a new therapeutic approach for Friedreich ataxia (FRDA), which can be done directly on the cells of a subject suffering from FRDA. This approach is based on the permanent removal of the GAA repeats in intron 1 of the FXN gene, which are responsible for FXN gene silencing. By generating additional mutations (e.g., deletions) by cutting upstream and downstream of the endogenous GAA repeat extension, preferably within intron 1 of the FXN gene, it is possible to permanently remove the pathological GAA repeats. Removal of all or part of the GAA repeat sequence within the endogenous FXN gene allows increasing FXN expression above the baseline level of FXN expression generated from the endogenous unmodified FXN gene comprising the original number of GAA repeats. Thus, by targeting polynucleotide sequences close to (e.g., upstream and/or downstream) of the GAA repeats, it is possible to remove the trinucleotide repeat extension in the FXN gene in cells to produce a mutated FXN gene and to increase FXN protein expression to levels above that observed in cells comprising the unmodified FXN gene comprising a pathological number of GAA trinucleotide repeats.

Applicants describe herein the use of the CRISPR system, using either S. pyogenes Cas9 (SpCas9), S. aureus Cas9 (SaCas9) and C. jejuni Cas9 (CjCas9) in combination with a pair of gRNAs, to delete GAA trinucleotide repeats in vitro in YG8R (25) and YG8sR (28) mice fibroblasts and in vivo in YG8R-mice. The YG8sR mouse model constitutes the in vivo model of choice to establish the possibility of editing the FXN gene in FRDA cells since it has only one copy of the human FRDA FXN transgene. Applicants have used the YG8sR mouse model to correct the FXN gene using an AAV coding for the SaCas9 and two gRNAs targeting sequences located upstream and downstream of the GAA repeats in intron 1 of the FXN gene. CRISPR nuclease/gRNAs combinations were also found to be effective in human FRDA cells in in vitro assays. Furthermore, Applicants have found that certain regions of intron 1 of the FXN gene are more easily targeted and cleaved than others by CRISPR nucleases (e.g., SpCas9, SaCas9 and CjCas9), making the deletion of GAA expansion more effective.

Accordingly, in an aspect, the present invention provides a method of modifying within a cell, a FXN gene comprising a plurality of GAA trinucleotide repeats in an intron of the gene, the method comprising: (a) introducing a first cut within the intron of the FXN gene creating a first intron end, wherein the first cut is located upstream of at least one GAA trinucleotide repeat of the plurality of GAA trinucleotide repeats; (b) introducing a second cut within the intron of the FXN gene creating a second intron end, wherein the second cut is located downstream of the at least one GAA trinucleotide repeat of the plurality of GAA trinucleotide repeats. Upon ligation of the first and second intron ends (preferably by NHEJ), the FXN gene is modified and some or all of the GAA trinucleotide repeats are removed. Removal of the GAA repeat expansion (in whole or in part) in FRDA cells increases FXN expression above the base level of FXN expression in the unmodified FRDA cells (i.e., having the corresponding unmodified GAA repeat expansion). In embodiments, the method is an in vitro method.

The present invention further provides a method of modifying within a cell, a FXN gene comprising a plurality of GAA trinucleotide repeats in an intron of the gene, the method comprising: (a) introducing a first cut within the intron of the FXN gene creating a first intron end, wherein the first cut is located upstream of or within the plurality of GAA trinucleotide repeats; (b) introducing a second cut within the intron of the FXN gene creating a second intron end, wherein the second cut is located downstream of or within the plurality of GAA trinucleotide repeats. Upon ligation of the first and second intron ends (preferably by NHEJ), the FXN gene is modified and some or all of the GAA trinucleotide repeats are removed. Removal of the GAA repeat expansion (in whole or in part) in FRDA cells increases FXN expression above the base level of FXN expression in the unmodified FRDA cells (i.e., having the GAA repeat expansion). In embodiments, the method is an in vitro method.

In embodiments, a method described herein allows for the correction of at least one allele of the FXN gene in a cell. In embodiments, the method allows for the correction of both alleles of the FXN gene in a cell.

In embodiments, the first and second cuts are introduced by providing a cell with (i) at least one CRISPR nuclease; and (ii) a pair of gRNAs consisting of a) a first gRNA which binds to a polynucleotide sequence within the intron of the FXN gene located upstream of at least one GAA trinucleotide repeat of the plurality of GAA trinucleotide repeats for introducing a first cut; (b) a second gRNA which binds to a polynucleotide sequence within the intron of the FXN gene located downstream of the at least one GAA trinucleotide repeat of the plurality of GAA trinucleotide repeats for introducing the second cut.

In embodiments, the first gRNA has a target sequence adjacent to a NGG (e.g., SpCas9) PAM nucleotide sequence corresponding to the following nucleotide positions: (a) nts 6579-6577; (b) nts 6592-6594; (c) nts 6543-6541; (d) nts 6670-6672; (e) nts 6645-6643; (f) nts 6647-6649; (g) nts 6202-6200; (h) nts 6103-6105; (i) nts 6221-6223; or (j) nts 6264-6262, wherein the nucleotide positions are given with respect to the FXN polynucleotide gene sequence set forth in GenBank NG_00845 (SEQ ID NO: 4). In embodiments, the second gRNA has a target sequence adjacent to a NGG PAM nucleotide sequence corresponding to the following nucleotide positions: (k) nts 6761-6759; (I) nts 6832-6834; (m) nts 6888-6886; (n) nts 6853-6851; (o) nts 6766-6768; (p) nts 6872-6874; (q) nts 7232-7230; (r) nts 7324-7326; (s) nts 7336-7334; or (t) nts 7142-7141, wherein the nucleotide positions are given with respect to the FXN polynucleotide gene sequence set forth in GenBank NG_00845 (SEQ ID NO: 4)

In embodiments, the first gRNA comprises (or consists of) a target sequence adjacent to a NNGRRT (e.g., SaCas9) PAM nucleotide sequence corresponding to the following nucleotide positions: (a) nts 6569-6574; (b) nts 6635-6640; or (c) nts 6691-6686, wherein the nucleotide positions are given with respect to the FXN polynucleotide gene sequence set forth in GenBank NG_00845 (SEQ ID NO: 4). In embodiments, the second gRNA comprises (or consists of) a target sequence adjacent to a NNGRRT PAM nucleotide sequence corresponding to the following nucleotide positions: (d) nts 6789-6784; (e) nts 7078-7073; or (f) nts 7158-7163, wherein the nucleotide positions are given with respect to the FXN polynucleotide gene sequence set forth in GenBank NG_00845 (SEQ ID NO: 4).

In embodiments, the first gRNA has a target sequence adjacent to a CjCas9 PAM (5′ NNNNRYAC, 5′-NNNVRYAC or 5′-NNNNACAC) nucleotide sequence corresponding to the following nucleotide positions: (a) nts 6400-6393; (b) nts 6411-6404; (c) nts 6464-6471; (d) nts 6501-6494; or (e) nts 6520-6513; wherein the nucleotide positions are given with respect to the FXN polynucleotide gene sequence set forth in GenBank NG_00845 (SEQ ID NO: 4). In embodiments, the second gRNA comprises (or consists of) a target sequence adjacent to a NNGRRT PAM nucleotide sequence corresponding to the following nucleotide positions: (f) nts 7062-7055; (g) nts 6980-6973; (h) nts 7032-7039; (i) nts 7041-7034; or (j) nts 7085-7078.

In embodiments, the first gRNA has a target sequence which is comprised between nts 6201 and 6633 of intron 1 of the FXN gene (e.g., set forth in SEQ ID NO: 4 (Acc. No. NG_008845)). In embodiments, the first gRNA has a target sequence which is comprised in a subregion between nts 6594 and 6633 of intron 1 of the FXN gene (e.g., set forth in SEQ ID NO: 4 (Acc. No. NG_008845)). In embodiments, the second gRNA has a target sequence which is comprised between nts 7078 and 7161 of intron 1 of the FXN gene (e.g., set forth in SEQ ID NO: 4 (Acc. No. NG_008845)). In embodiments, the second gRNA has a target sequence which is comprised in a subregion between nts 6973 and 7163 of intron 1 of the FXN gene (e.g., set forth in SEQ ID NO: 4 (Acc. No. NG_008845)).

In embodiments, the first and second gRNAs correspond to a pair of gRNAs set forth in Table 3. In embodiments, the first and second gRNAs correspond to a pair of gRNAs which is (i) C1/C11, (ii) C2/C11, (iii) C1/C20, (iv) C2/C20, (v) C15/C18, (vi) C15/C20, (vii) C16/C18, (viii) C16/C20, (ix) AC1/AC6, (x) AC2/AC6, (xi) AC3/AC6, (xii) CjJ1J7, (xiii) CjJ1J10, (xiv) CjJ2J7, (xv) CjJ2J10, (xvi) CjJ3J7, (xvii) CjJ3J10, (xviii) CjJ4J7, (xix) CjJ4J10, (xx) CjJ5J7, (xxi) CjJ5J10, wherein the gRNAs are listed in Table 5, 6 or 7. In embodiments, the pair of gRNAs is (iv) C2/C20, (vi) C15/C20, (viii) C16/C20, (xviii) CjJ4J7, or (xix) CjJ4J10.

In embodiments, the first gRNA and the second gRNA have a target sequence comprising at least 17 consecutive nucleotides of a target sequence set forth in Table 5, Table 6 or Table 7 or an allelic variant thereof. In embodiments, the first gRNA and the second gRNA are selected from the gRNAs listed in Table 5, 6, 7 or 8.

In embodiments, the number of nucleotides removed on each side of the GAA trinucleotide repeats does not exceed about 920 nucleotides in total. In embodiments, the number of nucleotides removed on each side of the GAA trinucleotide repeats is as set forth in Table 3.

In a further aspect, the present invention provides a gRNA pair for deleting a plurality of endogenous GAA trinucleotide repeats within an intron of a FXN gene within a cell, wherein the pair consists of a first gRNA and a second gRNA, wherein (a) the first gRNA binds to (the opposite strand of) a first polynucleotide sequence within the intron of the FXN gene located upstream of at least one GAA trinucleotide repeat of the plurality of GAA trinucleotide repeats for introducing a first cut; and (b) the second gRNA binds to (the opposite strand of) a second polynucleotide sequence within the intron of the FXN gene located downstream of the at least one GAA trinucleotide repeat of the plurality of GAA trinucleotide repeats for introducing a second cut.

In embodiments, the first cut introduced by a gRNA pair of the present invention is within about 650 nucleotides upstream of the GAA trinucleotide repeats. In embodiments, the first cut introduced by a gRNA pair of the present invention is within about 550 nucleotides upstream of the GAA trinucleotide repeats and the second cut is within about 550 nucleotides downstream of the GAA trinucleotide repeats. In embodiments, the first cut introduced by a gRNA pair of the present invention is within 506 nucleotides upstream of the GAA trinucleotide repeats and the second cut is within 478 nucleotides downstream of the GAA trinucleotide repeats. In embodiments, the first cut introduced by a gRNA pair of the present invention, is between 506 nucleotides and 30 nucleotides upstream of the GAA trinucleotide repeats and the second cut is between 478 nucleotides and 20 nucleotides downstream of the GAA trinucleotide repeats. In embodiments, the first and second cuts introduced by a gRNA pair of the present invention and the number of nucleotides removed in 5′ and 3′ of the GAA repeats is selected from those set forth in Table 3.

In embodiments, the first cut from the first gRNA removes between 30 and 625 nucleotides upstream the GAA trinucleotide repeats. In embodiments, the second cut from the second gRNA removes between 20 and 597 nucleotides downstream of the GAA trinucleotide repeats.

In embodiments, the second cut introduced by gRNAs of the present invention is within about 650 nucleotides downstream of the GAA trinucleotide repeats.

In embodiments, the first gRNA of the gRNA pair has a target sequence which is comprised between nts 6201 and 6633 of intron 1 of the FXN gene (e.g., set forth in SEQ ID NO: 4 (Acc. No. NG_008845)). In embodiments, the first gRNA of the gRNA pair has a target sequence which is comprised in a subregion between nts 6594 and 6633 of intron 1 of the FXN gene (e.g., set forth in SEQ ID NO: 4 (Acc. No. NG_008845)). In embodiments, the second gRNA of the gRNA pair has a target sequence which is comprised between nts 7078 and 7161 of intron 1 of the FXN gene (e.g., set forth in SEQ ID NO: 4 (Acc. No. NG_008845)). In embodiments, the second gRNA of the gRNA pair has a target sequence which is comprised in a subregion between nts 6973 and 7163 of intron 1 of the FXN gene (e.g., set forth in SEQ ID NO: 4 (Acc. No. NG_008845)). In embodiments, the target sequence of the first gRNA and/or second gRNA in the gRNA pair is selected from a subregion shown in FIG. 18.

In embodiments, the first cut from the first gRNA of the gRNA pair is between nts 6201 and 6633 of intron 1 of the FXN gene (e.g., set forth in SEQ ID NO: 4 (Acc. No. NG_008845)). In embodiments, the first cut from the first gRNA of the gRNA pair has a target sequence which is comprised in a subregion between nts 6594 and 6633 of intron 1 of the FXN gene (e.g., set forth in SEQ ID NO: 4 (Acc. No. NG_008845)). In embodiments, the second cut from the second gRNA of the gRNA pair has a target sequence which is comprised between nts 7078 and 7161 of intron 1 of the FXN gene (e.g., set forth in SEQ ID NO: 4 (Acc. No. NG_008845)). In embodiments, the second cut of the second gRNA of the gRNA pair has a target sequence which is comprised in a subregion between nts 6973 and 7163 of intron 1 of the FXN gene (e.g., set forth in SEQ ID NO: 4 (Acc. No. NG_008845)).

In embodiments, the gRNA pair of the present invention comprises: a first gRNA having a target sequence adjacent to a NGG PAM nucleotide sequence corresponding to the following nucleotide positions: (a) nts 6579-6577; (b) nts 6592-6594; (c) nts 6543-6541; (d) nts 6670-6672; (e) nts 6645-6643; (f) nts 6647-6649; (g) nts 6202-6200; (h) nts 6103-6105; (i) nts 6221-6223; or (j) nts 6264-6262, wherein the nucleotide positions are given with respect to the FXN polynucleotide gene sequence set forth in GenBank NG_00845 (SEQ ID NO: 4). In embodiments, the gRNA pair of the present invention comprises a second gRNA having a target sequence adjacent to a NGG PAM nucleotide sequence corresponding to the following nucleotide positions: (k) nts 6761-6759; (I) nts 6832-6834; (m) nts 6888-6886; (n) nts 6853-6851; (o) nts 6766-6768; (p) nts 6872-6874; (q) nts 7232-7230; (r) nts 7324-7326; (s) nts 7336-7334; or (t) nts 7142-7141, wherein the nucleotide positions are given with respect to the FXN polynucleotide gene sequence set forth in GenBank NG_00845 (SEQ ID NO: 4).

In embodiments, the first gRNA in the gRNA pair of the present invention comprises (or consists of) a target sequence adjacent to a NNGRRT PAM nucleotide sequence corresponding to the following nucleotide positions: a) nts 6569-6574; (b) nts 6635-6640; or (c) nts 6691-6686, wherein the nucleotide positions are given with respect to the FXN polynucleotide gene sequence set forth in GenBank NG_00845 (SEQ ID NO: 4).

In embodiments, the second gRNA in the gRNA pair of the present invention comprises (or consists of) a target sequence adjacent to a NNGRRT PAM nucleotide sequence corresponding to the following nucleotide positions: (d) nts 6789-6784; (e) nts 7078-7073; or (f) nts 7168-7163, wherein the nucleotide positions are given with respect to the FXN polynucleotide gene sequence set forth in GenBank NG_00845 (SEQ ID NO: 4).

In embodiments, the first gRNA in the gRNA pair of the present invention comprises (or consists of) a target sequence adjacent to a CjCas9 PAM (5′ NNNNRYAC, 5′-NNNVRYAC or 5′-NNNNACAC) nucleotide sequence corresponding to the following nucleotide positions: (a) nts 6400-6393; (b) nts 6411-6404; (c) nts 6464-6471; (d) nts 6501-6494; or (e) nts 6520-6513; wherein the nucleotide positions are given with respect to the FXN polynucleotide gene sequence set forth in GenBank NG_00845 (SEQ ID NO: 4). In embodiments, the second gRNA in the gRNA pair of the present invention comprises (or consists of) a target sequence adjacent to a NNGRRT PAM nucleotide sequence corresponding to the following nucleotide positions: (f) nts 7062-7055; (g) nts 6980-6973; (h) nts 7032-7039; (i) nts 7041-7034; or (j) nts 7085-7078.

In embodiments, the number of nucleotides removed on each side of the GAA trinucleotide repeats by the gRNA pair of the present invention does not exceed about 920 nucleotides in total.

In embodiments, the first and second gRNAs in the gRNA pair of the present invention correspond to a pair of gRNAs which is (i) C1/C11, (ii) C2/C11, (iii) C1/C20, (iv) C2/C20, (v) C16/C18, (vi) C16/C20, (vii) C16/C18, (viii) C16/C20, (ix) AC1/AC6, (x) AC2/AC6, (xi) AC3/AC6, (xii) CjJ1J7, (xiii) CjJ1J10, (xiv) CjJ2J7, (xv) CjJ2J10, (xvi) CjJ3J7, (xvii) CjJ3J10, (xviii) CjJ4J7, (xix) CjJ4J10,; (xx) CjJ5J7, or (xxi) CjJ5J10, wherein the gRNAs are listed in Table 5, 6 or 7. In embodiments, the gRNA is (iv) C2/C20, (vi) C16/C20, (viii) C16/C20, (xviii) CjJ4J7, or (xix) CjJ4J10.

In embodiments, the first gRNA and the second gRNA in the gRNA pair of the present invention have a target sequence comprising at least 17 consecutive nucleotides of a target sequence set forth in FIG. 18, or Table 5, Table 6 or Table 7 or an allelic variant thereof. In embodiments, the first gRNA and the second gRNA are selected from the gRNAs listed in FIG. 18, Tables 5, 6 and 7. In embodiments, the gRNA pair of the present invention comprises one more additional gRNAs.

Also provided is a nucleic acid comprising one or more polynucleotide sequences encoding one or both members of the gRNA pair of the present invention. In embodiments, the nucleic acid further comprises a sequence encoding one or more CRISPR nucleases.

Also provided is a nucleic acid comprising a modified FXN gene comprising ligated first and second intron ends generated by the gRNA pair of the present invention. In embodiments, the modified FXN gene comprises ligated first and second intron ends defined by the cut sites identified in Table 5, 6 or 7. In embodiments, the modified FXN gene comprises a polynucleotide sequence as set forth in FIG. 14 or 15 or any one of SEQ ID NO: 171-195, or an allelic variant thereof. In embodiments, the modified FXN gene comprises one or more nucleotide additions and/or deletions at position(s) corresponding to a nucleotide addition or deletion shown in FIG. 14 or 15, or an allelic variant thereof.

In embodiments, the present invention also concerns a vector comprising one or more of the above-noted nucleic acids. In embodiments, the vector comprises a first nucleic acid comprising a polynucleotide sequence encoding the first gRNA of the gRNA pair of the present invention, a second nucleic acid comprising a polynucleotide sequence encoding the second gRNA of the gRNA pair of the present invention and a third nucleic acid nucleic acid comprising a polynucleotide sequence encoding a CRISPR nuclease. In embodiments the promotor sequence for expressing the gRNA pair is different from the promoter sequence for expressing the CRISPR nuclease in the vector. In embodiments, the vector is a viral vector. In embodiments, the viral vector is an AAV or a Sendai virus derived vector. In embodiments, the AAV is an AAV-PHP.B, AAV-9 or AAV-DJ8 viral vector. In embodiments, the promoter sequence for expressing one or more gRNAs (or gRNA pair) of the present invention is a U6, Cbh or CMV promoter. In embodiments the CMV promoter comprises a deletion (212 CMV or 259 CMV).

Also provided is a combination of vectors encoding one or more gRNAs of the present invention and/or one or more CRISPR nucleases. In embodiments, the combination of vectors comprises: a first vector comprising a first nucleic acid comprising a polynucleotide sequence encoding the first gRNA of the gRNA pair of the present invention; and a second vector comprising a second nucleic acid comprising a polynucleotide sequence encoding the second gRNA of the gRNA pair of the present invention. In embodiments, the above vectors in the combination further encode one or more CRISPR nucleases. In embodiments, the combination of vectors further comprises a third vector comprising a third nucleic acid comprising a polynucleotide sequence encoding one or more CRISPR nucleases. In embodiments, the combination of vectors comprises: a gRNA vector comprising a first nucleic acid comprising a polynucleotide sequence encoding the first gRNA of the gRNA pair of the present invention and a second nucleic acid comprising a polynucleotide sequence encoding the second gRNA of the gRNA pair of the present invention; and a CRISPR nuclease vector comprising a third nucleic acid comprising a polynucleotide sequence encoding one or more CRISPR nucleases.

Also provided is a cell comprising one or both members of a gRNA pair, a nucleic acid, a vector, and/or a combination of vectors of the present invention.

The present invention further provides a composition comprising one or both members of a gRNA pair, a nucleic acid, a vector, a combination of vectors, and/or a cell of the present invention. In embodiments, the composition further comprises a biologically acceptable carrier, e.g., a pharmaceutically acceptable carrier.

The present invention also provides a kit comprising one or both members of the above-noted gRNA pair, above-noted nucleic acid, vector, combination of vectors, cell, composition, CRISPR nucleases and/or nucleic acids encoding one or more CRISPR nucleases. In embodiments, the kit further comprises instructions for modifying within a cell, a FXN gene comprising a plurality of GAA trinucleotide repeats in an intron of the gene, in accordance with the present invention. In embodiments, the kit is for use in treating Friedreich ataxia in a subject in need thereof.

The present invention also concerns a method for treating Friedreich ataxia in a subject, comprising modifying a FXN gene and increasing FXN expression within a cell of the subject in accordance with the method of the present invention.

The present invention also concerns a method for increasing FXN expression within a cell comprising a FXN gene comprising a plurality of GAA trinucleotide repeats in an intron of the gene, comprising modifying the FXN gene to remove some or all of the GM trinucleotide repeats in accordance with a method described herein.

The present invention further concerns a method for treating Friedreich ataxia in a subject, comprising contacting a cell of the subject with (i)(a) the above-described gRNA pair or one or more nucleic acids encoding the gRNA pair, and (b) one or more CRISPR nuclease polypeptides or nucleic acids encoding one or more CRISPR nuclease polypeptides; (ii) the above-noted vector or combination of vectors; and/or (iii) the above-noted composition of the present invention.

The present invention also concerns a use of (i)(a) the above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) the above-noted composition, for treating Friedreich ataxia in a subject.

The present invention also concerns a use of the (i)(a) the above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) above-noted composition, for the preparation of a medicament for treating Friedreich ataxia in a subject.

The present invention also concerns the (i)(a) above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) above-noted composition, for use in treating Friedreich ataxia in a subject.

The present invention also concerns the (i)(a) the above-noted gRNA pair or one or more nucleic acids encoding the gRNA pair, and (b) one or more CRISPR nuclease polypeptides or nucleic acids encoding one or more CRISPR nuclease polypeptides, (ii) above-noted vector or combination of vectors, and/or (iii) the above-noted composition for use in the preparation of a medicament for treating Friedreich ataxia in a subject.

The present invention further concerns a method for modifying within a cell, an FXN gene comprising a plurality of GAA trinucleotide repeats in an intron of said gene, comprising contacting the cell with (i)(a) the above-described gRNA pair or one or more nucleic acids encoding the gRNA pair, and (b) one or more CRISPR nuclease polypeptides or nucleic acids encoding one or more CRISPR nuclease polypeptides; (ii) the above-noted vector or combination of vectors; and/or (iii) the above-noted composition of the present invention, such that the FXN gene is modified to remove some or all of the GAA trinucleotide repeats. In an embodiment, the method is an in vitro method.

The present invention also concerns a use of (i)(a) the above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) the above-noted composition, for modifying within a cell, an FXN gene comprising a plurality of GAA trinucleotide repeats in an intron of said gene, comprising contacting the cell with (i)(a) the above-described gRNA pair or one or more nucleic acids encoding the gRNA pair, and (b) one or more CRISPR nuclease polypeptides or nucleic acids encoding one or more CRISPR nuclease polypeptides; (ii) the above-noted vector or combination of vectors; and/or (iii) the above-noted composition of the present invention, such that the FXN gene is modified to remove some or all of the GAA trinucleotide repeats.

The present invention also concerns the (i)(a) above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) above-noted composition, for use in modifying within a cell, an FXN gene comprising a plurality of GAA trinucleotide repeats in an intron of said gene, comprising contacting the cell with (i)(a) the above-described gRNA pair or one or more nucleic acids encoding the gRNA pair, and (b) one or more CRISPR nuclease polypeptides or nucleic acids encoding one or more CRISPR nuclease polypeptides; (ii) the above-noted vector or combination of vectors; and/or (iii) the above-noted composition of the present invention, such that the FXN gene is modified to remove some or all of the GAA trinucleotide repeats.

The present invention also concerns a use of (i)(a) the above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) above-noted composition, for increasing FXN expression within a cell comprising a FXN gene comprising a plurality of GAA trinucleotide repeats in an intron of the gene, whereby the FXN gene is modified to remove some or all of the GAA trinucleotide repeats in accordance with a method described herein.

The present invention also concerns the (i)(a) above-noted gRNA pair or one or more nucleic acids encoding the gRNA pair, and (b) one or more CRISPR nuclease polypeptides or nucleic acids encoding one or more CRISPR nuclease polypeptides, (ii) the above-noted vector or combination of vectors, and/or (iii) above-noted composition, for increasing FXN expression within a cell comprising a FXN gene comprising a plurality of GAA trinucleotide repeats in an intron of the gene, whereby the FXN gene is modified to remove some or all of the GAA trinucleotide repeats in accordance with a method described herein.

Also provided is a reaction mixture comprising (a) the above-noted gRNA pair or one or more nucleic acids encoding the gRNA pair, and (b) one or more CRISPR nuclease polypeptides or one or more nucleic acids encoding one or more CRISPR nuclease polypeptides.

In embodiments, the above-noted FXN gene comprises at least 70 GAA trinucleotide repeats within the intron. In embodiments, the above-noted FXN gene comprises at least 150 GAA trinucleotide repeats within the intron. In embodiments, the above-noted CRISPR nuclease comprises or consists of CjCas9, SaCas9 and/or SpCas9.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-D show CRISPR targeting of mutated GAA trinucleotide repeats in the FXN gene. (A) Regions (lines with black dots) targeted by SpCas9 gRNAs are identified in the pre- and post-GAA trinucleotide regions of the human FXN intron 1 and positions of the primers used (lines with squares). (B) The YG8R mouse fibroblasts contain two tandem copies of the human FXN transgene (from a FRDA patient), with about 82 and 190 GAA repeats, respectively. (C) Predicted F3/R3 PCR-amplified product lengths from extracted genomic DNA following transfection of YG8R cells with the SpCas9 gene and different pairs of gRNAs. (D) Screening of gRNA pairs in YG8R cells, using the F3/R3 primer set (see Tables 5 and 6 for details about target sequences; sequences removed and position of the cuts);

FIGS. 2A-E show deletion of GAA trinucleotide repeats in YG8R mouse fibroblasts. (A) F3/R3 PCR amplification of genomic DNA (gDNA) from YG8R fibroblasts transfected with plasmids coding for SpCas9_P2A_puromycin and gRNA pairs. The correction of the FXN gene was first detected in a heterogeneous (pooled) YG8R fibroblast population. Cells successfully transfected were then selected using the puromycin selection drug and expanded as individual clones. (B) Putative rearrangements of the FXN gene in YG8R fibroblasts following correction with a pair of gRNAs, i.e., one targeting a sequence located upstream (a or a′) and the other targeting a sequence located downstream (b or b′) of the GAA repeat expansion. A positive clone status (+) was given when no F3/R3 PCR-amplified band including a GAA repeat was seen on agarose gel. (C) Summary of the YG8R clonal expansion. Partial deletion corresponds to clones that were still having a F3/R3 PCR-amplified product containing GAA repeats. Complete deletion status was attributed to clones that did not contain any GAA repeat resulting from one of the rearrangements illustrated in (B) as a positive clone status. (D) Agarose gel showing F3/R3 PCR-amplified products corresponding to a complete deletion of both GAA repeat expansions (one in each FXN transgene) in the FRDA FXN genes in YG8R isolated clones (clones considered as positive in B). (E) Amplified F3/R3 products in (D) were sub-cloned and sequenced to detect junction points between the pre- and post- GAA repeat regions of intron 1 following correction (sequence regions shown: SEQ ID NOs 212-223). Boxes correspond to PAM sequences for SpCas9 while arrows show the expected cut site (see Tables 5 and 6 for details about target sequences; sequences removed and position of the cuts);

FIGS. 3A-B show PCR-amplified F3/R3 products for clone identification in gRNA/SpCas9_2A_Puro transfected YG8R cells. Genomic DNAs extracted from isolated YG8R clones were amplified using the F3/R3 primer set. The agarose gel analysis revealed three different patterns following NHEJ rearrangement in YG8R clones, following cuts by the SpCas9 and gRNAs targeting the pre- and post-GAA regions within FXN intron 1. Positive clones, i.e. those with a complete deletion of the GAA from both transgenes are annotated as “C”, those with a GAA deletion from one transgene out of two are annotated as “P” and those with no cut or ambiguous status are annotated negatives (−). (A) left Panel gRNA pair C2C20, right panel gRNA pair C15C20; and (B) gRNA pair C15C20 (see Tables 5 and 6 for details about target sequences; sequences removed and position of the cuts);

FIGS. 4A-C show protein expression and copy number analysis of CRISPR-edited YG8R fibroblasts. (A) Western blot protein analysis of YG8R cells transfected with different combinations of gRNAs and SpCas9. (B) Gene copy number analysis of some selected clones shown in (A). (C) Schematic representation of results obtained regarding putative rearrangements of corrected YG8R clones;

FIGS. 5A-C show FXN protein expression analysis of CRISPR-edited YG8R fibroblast clones. (A) Western blot protein analysis of the global YG8R cell population transfected with different combinations of gRNAs and SpCas9. (B) and (C) Western blot analysis of protein extracted from isolated YG8R clones;

FIGS. 6A-F show deletion of GAA trinucleotide repeats in YG8sR mouse fibroblasts. (A) Schematic representation of the human FXN transgene in YG8sR cells which comprise about 190 GAA repeats in intron 1. (B) F2/R3 PCR-amplified products containing GAA trinucleotide repeats showing differences between cells used in this study. Y47R cells contain a single copy of a normal human FXN transgene with approximately 9 GAA repeats. Fibroblast cell lines YG8sR-6, YG8sR-8 and YG8sR-39 have approximately 190 GAA repeats while YG8R cells contain two copies in tandem of the human FXN gene with approximately 82 and 190 GAA repeats respectively. (C) PCR-amplification of genomic DNA of YG8sR-39 cells transfected with a C2C20 or C15C20 gRNA pair and SpCas9_P2A_puromycin using the F3/R3 primer set. YG8sR-39 cells were amplified as clones following this experiment. PURO represents cells transfected with a plasmid encoding the SpCas9-only (no gRNA). (D) Putative rearrangement of the single copy of the FRDA FXN gene in YG8sR fibroblasts following deletion of the GAA repeats using a pair of gRNAs targeting sequences upstream (a) and downstream (b) the GAA repeat expansion. A positive clone status (+) was given when no F3/R3 PCR-amplified band corresponding intron 1 sequences comprising GAA repeats were seen on agarose gel. (E) Summary of YG8sR clonal expansion. (F) Agarose gel showing F3/R3 PCR-amplified products corresponding to the corrected FXN gene from YG8sR isolated clones C2C20-13 and -20. Similar results were obtained for C2C20-15 and -18 clones;

FIGS. 7A-D show protein and mRNA expression analysis of CRISPR-edited YG8sR fibroblasts. (A) Western blot protein analysis of YG8sR clones treated with the C2C20 gRNA pair or a vector expressing SpCas9/PURO but no gRNA (negative control). (B) Quantification of FXN protein expression in four (n=4) different protein extractions from YG8sR cells treated with the C2C20 gRNA pair and corresponding control samples. (C) FXN mRNA expression analysis of total RNA extracted from YG8sR cells treated or not with the C2C20 gRNA pair. Three (n=3) different RNA extractions were made for each condition. Human FXN transgene expression was monitored by qRT-PCR using primers to amplify hFXN exon2/3 and 5′UTR/exon1 as previously published (51) (see also Table 4 in Example 1). (D) Gene copy number analysis of selected YG8sR clones;

FIGS. 8A-D show genomic DNA analysis of YG8sR clones. YG8sR C2C20 corrected clones were analyzed using different pairs of primers (for primer sequences, see Table 4 in Example 1) to determine their genomic organization. (A) Schematic representation of the human FXN transgene in YG8sR cells showing the relative position of the primers within intron or exon sequences. (B) PCR-amplification of genomic DNA using the F4/R10 primer pair. (C) PCR-amplification of genomic DNA using the F9/R9 primer pair. (D) PCR-amplification of genomic DNA using the F10/R10 primer pair;

FIGS. 9A-B show the in vivo electroporation of SpCas9 and gRNAs encoding plasmids into YG8R mouse model. (A) Schematic representation of electroporation experiment. (B) F2/R3 PCR-amplified products obtained following genomic DNA extraction from Tibialis anterior (TA) samples of YG8R mice treated with SpCas9/gRNAs encoding plasmids. Mouse#/side refers to the individual mouse number and its right (R) or left (L) TA. “&” represents the expected size of the amplification product following removal of GAA repeats in the FXN gene with the 016020 gRNA combination. “*” represents the expected size of the amplification product following removal of GAA repeats in the FXN gene with the C2C20 gRNA combination. “†” identifies the expected size of the amplification product for the unique uncut FXN gene in YG8LR cells;

FIGS. 10A-E show removal of the GAA trinucleotide repeats using the S. aureus Cas9 (SaCas9) nuclease. (A) Target regions for S. aureus Cas9 (which uses a NNGRRT sequence as a PAM) were identified, in the pre- and post-GAA trinucleotide regions of FXN intron 1 (AC1, AC2, AC3 and AC6). (B) Schematic representation of the modifications introduced in the original px601 plasmid (see Example 1 for details). Briefly, a polynucleotide encoding an additional U6 or H1m promoter and a SaCas9 tracrRNA were added to allow cloning of a second gRNA within the same plasmid. The CMV promoter was then shortened to 259 or 212 bp. (C) F3/R3 PCR-amplified products showing effectiveness of the correction using combinations of gRNA and the SaCas9 protein in YG8sR fibroblasts. (D) F2/R3 and F3/R3 PCR-amplified products showing the effects of the correction in YG8sR using the gRNA pair AC2 and AC6 expressed from different promoters (either U6 or Him). The SaCas9 was expressed under the control of a truncated (212 or 259) or WT form of the CMV promoter. (E) Western blot showing protein expression of SaCas9 expressed from SaCas9-CMV (WT, 212, 259) or SpCas9-CBh promoters using respectively anti-HA or anti-FLAG antibodies;

FIG. 11 shows that single intravenous injection of AAV vectors coding for SpCas9 and gRNAs (02020 combination) enables correction of intron 1 of the FXN gene in liver cells. (A) Adeno-Associated virus (AAV) vector design used in this experiment. (B) Bar graph of percentage of correction (fraction abundance) in liver cells of YG8sR treated mice. Each bar represents an average of 2-4 ddPCR replicate reads. A PCR gel analysis of the presence of the AAV-Cas9 and or AAV-gRNA in liver samples using primers targeting vector is shown (see Example 1 and Example 9 for details);

FIG. 12 shows removal of GAA trinucleotide repeats from the FXN gene intron 1 in human FRDA primary fibroblasts. C2C20 or C15C20 gRNA combinations and the SpCas9 were nucleofected in human FRDA primary fibroblasts either as plasmids (DNA) or a mixture of SpCas9 recombinant protein and gRNAs (RNA+prot). Cells were also nucleofected only with the Cas9 protein (Cas9p) or buffer (NT) as negative controls. All FRDA patients (n=3) have a different of GAA repeats (see Material and method and Example 10 for details);

FIG. 13 shows the nucleotide sequence of intron 1 (+strand) of the FXN gene. Intron 1 of the FXN gene extends from nts 5644 to 15822 of NG_008845 (SEQ ID NO: 4) and comprises 10179 nts. This polynucleotide sequence comprises six (6) GAA repeats (boxed) from nts 6725 to 6742 of NG_008845. Exemplary gRNA target sequences are shown. Nucleotides shown in bold represent gRNAs target sequences on the complementary (−) strand of NG_008845 (C13, C16, C3, C1, AC2, C5, SaC3, C7, C9, AC4, C10, AC5, C20, C17 and C19). Underlined sequences represent target sequences of gRNAs located on the (+) strand (C14, C15, AC1, C2, C6, C4, C11, C8, C12, AC6 and C18). AC1-AC6 sequences represent gRNA target sequences recognized by S. aureus Cas9 (i.e. sequences adjacent to a PAM corresponding to NNGRRT (wherein R is A or G)). See Tables 5 and 6 for information of the gRNAs identified on the figure;

FIGS. 14A-D show partial polynucleotide sequencing results of corrected FXN gene using exemplary gRNA combinations of the present invention. The last nucleotide of the pre-GAA repeats cut (upstream cut) is underlined and the first nucleotide of the post GAA repeats cut (downstream cut) is shown in bold. Inserted nucleotides are shown in italic. Deleted nucleotides are shown between [ ]. (A) C15C20 gRNA combination (B); C2C11 gRNA combination; (C) C2C20 gRNA combination; and (D) 016C20 gRNA combination;

FIGS. 15A-E show partial corrected FXN polynucleotide gene sequences using exemplary gRNA combinations of the present invention. (A)C15C18; (B) C16C18; (C) C1C20; (D) AC1AC6; and (E) AC2AC6;

FIGS. 16A-B show that CjCas9 is as efficient as SpCas9 to generate deletion of GAA repeats. (A) Schematic representation of Cas9 orthologs tested herein (modified from Kim, E. Nat Commun (2017)). (B) 293T cells were transfected with pRGEN-CMV-CjCas9 plasmid (Addgene #89752) and two guides expressed individually from the pU6-Cj- gRNA plasmid (Addgene #89753). Cells were harvested at 72 hours and PCR amplification was performed on genomic DNA using F1 and R3 primers (see Table 4) to amplify edited (lower band, without GAA) and uncut sequences. Most efficient combinations were used in this experiment but all selected gRNA worked to some extent. All pre-GAA gRNAs (Cj1-Cj5) worked in combination with post-GAA gRNAs Cj7 or Cj10 but some better than others. Corresponding results were obtained in YG8sR cells (not shown). Expected bands were obtained for non-edited molecules (1507 bp), sg1/7 (927 bp), sg1/10, (822 bp) sg2/7 (938bp), sg2/10 (833 bp), sg3/7 (984 bp), sg3/10 (879 bp), sg4/7 (1020 bp), sg4/10 (920 bp), sg5/7 (1047 bp) and sg5/10 (942 bp) (see Table 7 for details about target sequences; sequences removed and position of the cuts);

FIGS. 17A-B show that the use of a single vector for providing Cas9 and a gRNA pair is efficient to edit the FXN gene and remove GAA repeats. (A) Single vector design includes the CjCas9 gene (with SV40 NLS and HA tag) under the control of a CBh promoter, a SV40 late polyA and short WPRE (Woodchuck Hepatitis Virus (WHP) Post Transcriptional Regulatory Element) sequences, as well as two gRNAs under the control of either the human U6 or the H1 minimal promoter. (B) 293T cells were transfected with three plasmids (3V):pRGEN-CMV-CjCas9 (lanes 1-3), pU6-Cj- gRNA4 (lanes 2-3) and pU6-Cj- gRNA7 (lane 2) or gRNA10 (lane 3). Cells were also transfected with one plasmid (1V) either containing no guides (lane 4), gRNA 4 and 17 (lane 5) or gRNA 4 and 10 (lane 6). Cells were harvested at 72 hours and PCR amplification was performed on gDNA using F1 and R3 primers to amplify edited (lower band, without GAA) and uncut sequences. Expected bands were obtained for uncut (1507 bp) or edited sg4/7 (1020 bp) and sg4/10 (920 bp) PCR products (see Table 7 for details about target sequences; sequences removed and position of the cuts); and

FIG. 18 shows the most effective regions on intron 1 of the FXN gene for targeting gRNAs and CRISPR nucleases and deleting GAA repeats. (A) Schematic representation of FXN intron 1 and targeted gRNAs for SpCas9. (B) Schematic representation of FXN intron 1 and targeted gRNAs for SaCas9. (C) Schematic representation of FXN intron 1 and targeted gRNAs for CjCas9. Particularly effective regions on FXN intron 1 for targeting gRNAs and cutting upstream (6201-6633, SEQ ID NO: 209) and downstream (7078-7161, SEQ ID NO: 10) of GAA repeats are shown at the bottom of the figure.

### DESCRIPTION OF ILLUSTRATIVE EMBODIMENTS

ciRNAs

In order to cut DNA at a specific site, CRISPR nucleases require the presence of a gRNA and a protospacer adjacent motif (PAM), which immediately follows (or precedes) the gRNA target sequence in the targeted polynucleotide gene sequence. The PAM is located at one end (i.e., the 3′ end or 5′ end) of the gRNA target sequence but is not part of the gRNA guide sequence. Different CRISPR nucleases require a different PAM. Accordingly, selection of a specific polynucleotide target sequence (e.g., in the FXN gene nucleic acid sequence) by a gRNA is generally based on the CRISPR nuclease used. The PAM for the Streptococcus pyogenes Cas9 CRISPR system is 5′-NRG-3′, where R is either A or G, and characterizes the specificity of this system in human cells. The S. pyogenes Type II system naturally prefers to use an “NGG” sequence, where “N” can be any nucleotide, but also accepts other PAM sequences, such as “NAG” in engineered systems. The PAM of S. aureus is NNGRR (or NNGRRT wherein R is A or G). Similarly, the Cas9 derived from Neisseria meningitides (NmCas9) normally has a native PAM of NNNNGATT, but has activity across a variety of PAMs, including a highly degenerate NNNNGNNN PAM. Another Example is the Cas9 derived from Campylobacter jejuni (CjCas9) which is advantageously small and which generally recognizes a NNNNRYAC PAM (but also a “NNNVRYAC” or “NNNNACAC), where “N” can be any nucleotide, ““R” is a purine (G or A), and “Y” is a pyrimidine (T or C). CjCas9 also recognizes “NNNVRYAC” or “NNNNACAC PAM, where “V” is A, G or C.

In a preferred embodiment, the PAM for a Cas9 or Cpf1 protein used in accordance with the present invention is a NGG (SpCas9), a NNGRRT (SaCas9), a NNNNRYAC, NNNVRYAC or NNNNACAC (CjCas9) or TTTN (AsCpf1 and LbCpf1) nucleotide-sequence. Table 1 below provides a list of non-limiting examples of CRISPR/nuclease systems with their respective PAM sequences.

TABLE 1
Non-exhaustive list of CRISPR-nuclease systems from different species (see. Mohanraju, P. et al.
(58); Shmakov, S et al. (59); Zetsche, B. et al (60); and Shah et al., (63)). Also included are examples of engineered
variants recognizing alternative PAM sequences (see Kleinstiver, BP. et al., (61) and (62)).

| CRISPR nuclease/subtype | PAM Sequence | Cut site |
|---|---|---|
| Streptococcus pyogenes (SP); SpCas9 | NGG + NAG (in 3′) | Blunt end; 3-4bp upstream of the PAM |
| (subtype II) | | sequence |
| SpCas9 D1135E variant (subtype II) | NGG (in 3′, reduced NAG binding) | Blunt end; 3-4bp upstream of the PAM |
| | | sequence |
| SpCas9 VRER variant (subtype II) | NGCG (in 3′) | Blunt end; 3-4bp upstream of the PAM |
| | | sequence |
| SpCas9 EQR variant (subtype II) | NGAG (in 3′) | Blunt end; 3-4bp upstream of the PAM |
| | | sequence |
| SpCas9 VQR variant (subtype II) | NGAN or NGNG (in 3′) | Blunt end; 3-4bp upstream of the PAM |
| | | sequence |
| Staphylococcus aureus (SA); SaCas9 | NNGRRT or NNGRR(N), (in 3′) (R = A | Blunt end; 3-4bp upstream of the PAM |
| (subtype II) | or G) | sequence |
| SaCas9 KKH variant (subtype II) | NNNRRT (in 3′) (R = A or G) | Blunt end; 3-4bp upstream of the PAM |
| | | sequence |
| Neisseria meningitidis (NM) | NNNNGATT (in 3′) | Blunt end; 3-4bp upstream of the PAM |
| | | sequence |
| AsCpf1 | TTTN (in 5′) | 5 nucleotide 5′ overhang 18-23 bases |
| | | away from the PAM. |
| LbCpf1 | TTTN (in 5′) | 5 nucleotide 5′ overhang 18-23 bases |
| | | away from the PAM. |
| Campylobacter jejuni (Cj) | NNNNRYAC, NNNVRYAC, or | Blunt end; 3-4bp upstream of the PAM |
| | NNNNACAC (in 3′) | sequence |

Other non-limiting examples of known CRISPR nucleases that may be used include CRISPR nucleases from Streptococcus thermophilus (subtype II-A, PAM: NNAGAAW (in 3′) (W=A or T); Treponema denticola (PAM: NAAAAC (in 3′); Streptococcus agalactiae (PAM: NGG (in 3′)); Sulfolobus solfataricus (subtype I-Al, PAM: CNN); Sulfolobus solfataricus (subtype I-A2, PAM: TCN); Haloquadratum walsbyi (subtype I-B, PAM: TTC), Escherichia coli (subtype I-E, PAM: AWG); Escherichia coli (subtype I-F; PAM: CC); and Pseudomonas aeruginosa (subtype I-F, PAM: CC).

As used herein, the expression “gRNA” (which is used interchangeably with “sgRNA”) refers to a guide RNA which in an embodiment is a fusion between the gRNA guide sequence (or CRISPR targeting RNA or crRNA) and the CRISPR nuclease recognition sequence (tracrRNA). It provides both targeting specificity and scaffolding/binding ability for the CRISPR nuclease of the present invention. Alternatively, the gRNA may be provided as two separate entities (a tracrRNA and a gRNA guide sequence (i.e., target-specific sequence/crRNA)). gRNAs of the present invention do not exist in nature, i.e., they are non-naturally occurring nucleic acid(s).

A “target region”, “target sequence” or “protospacer” in the context of gRNAs and CRISPR system of the present invention are used herein interchangeably and refers to the region of the target gene which is targeted by the CRISPR/nuclease-based system, without the PAM. It refers to the sequence corresponding to the nucleotides that are adjacent to the PAM (i.e., in 5′ or 3′ of the PAM, depending of the CRISPR nuclease) in the genomic DNA. It is the DNA sequence that is included into a gRNA expression construct (e.g., vector/plasmid/AW). The CRISPR/nuclease-based system may include at least one (i.e., one or more) gRNAs, wherein each gRNA targets a different DNA sequence on the target gene. The target DNA sequences may be overlapping. The target sequence or protospacer is followed or preceded by a PAM sequence at an end (3′ or 5′ depending on the CRISPR nuclease used) of the protospacer. Generally, the target sequence is immediately adjacent (i.e., is contiguous) to the PAM sequence (it is located on the 5′ end of the PAM for SpCas9-like nuclease and at the 3′ end for Cpf1-like nuclease).

As used herein, the expression “gRNA guide sequence” refers to the corresponding RNA sequence of the “gRNA target sequence”. Therefore, it is the RNA sequence equivalent of the protospacer on the target polynucleotide gene sequence. It does not include the corresponding PAM sequence in the genomic DNA. It is the sequence that confers target specificity. The gRNA guide sequence is preferably linked to a CRISPR nuclease recognition sequence (transactivating CRISPR RNA, i.e., tracrRNA, scaffolding RNA) which binds to the nuclease (e.g., Cas9/Cpf1). Although it is advantageous that the tracrRNA sequence and gRNA guide sequence be provided as a single RNA, it is also possible to provide the tracrRNA as a separate entity. The gRNA guide sequence recognizes and binds to the targeted gene of interest. It hybridizes with (i.e., is complementary to) the opposite strand of a target gene sequence, which comprises the PAM (i.e., it hybridizes with the DNA strand opposite to the PAM). As noted above, the “PAM” is the nucleic acid sequence, that immediately follows (is contiguous to) the target sequence in the target polynucleotide but is not in the gRNA.

A “CRISPR nuclease recognition sequence” (e.g., Cas9/recognition sequence) refers to the portion of the gRNA guide sequence that binds to the CRISPR nuclease (tracrRNA, scaffolding RNA or other recognition sequence (e.g., SEQ ID NOs: 91 (SpCas9), 93 (SaCas9), 154, and 94 (Cpf1)). It leads the CRISPR nuclease to the target sequence so that it may bind and cut the target nucleic acid. It is adjacent the gRNA guide sequence (in 3′ (e.g., Cas9) or 5′ (Cpf1) depending on the CRISPR nuclease used). In embodiments, the CRISPR nuclease recognition sequence is a Cas9 recognition sequence having at least 65, 74, 76 or 77 nucleotides. In embodiments, the CRISPR nuclease recognition sequence is a Cpf1 recognition sequence (5′ direct repeat) having about 20 nucleotides. In particular embodiments, the Cas9 recognition sequence (gRNA scaffold sequence derived from crRNA and tracrRNA-) comprises (or consists of) the sequence as set forth in SEQ ID NO: 92, 93 or 154. The gRNA of the present invention may comprise any variant of this sequence, provided that it allows for the binding of the CRISPR nuclease protein of the present invention to the FXN gene. In embodiments, the CRISPR nuclease (e.g., Cas9 or Cpf1) recognition sequence is a CRISPR nuclease recognition sequence having at least 65 nucleotides. In embodiments, the CRISPR nuclease recognition sequence is a CRISPR nuclease recognition sequence having at least 74, 76 or 77 nucleotides.

As noted above not all CRISPR nucleases require a tracrRNA to function. Cpf1 is a single crRNA-guided endonuclease. Unlike Cas9, which requires both an RNA guide sequence (crRNA) and a tracrRNA (or a fusion or both crRNA and tracrRNA) to mediate interference, Cpf1 processes crRNA arrays independent of tracrRNA, and Cpf1-crRNA complexes alone cleave target DNA molecules, without the requirement for any additional RNA species (see Zetsche et al. (60)). Therefore, in the case of Cpf1, the CRISPR recognition sequence only comprises the conserved portion of the crRNA (i.e., without the target sequence).

In embodiments, the gRNA may comprise a “G” at the 5′ end of its polynucleotide sequence. The presence of a “G” in 5′ is preferred when the gRNA is expressed under the control of the U6 promoter (Koo T. et al. (65)). The CRISPR/nuclease system of the present invention may use gRNAs of varying lengths. The gRNA may comprise a gRNA guide sequence of at least 10 nts, at least 11 nts, at least a 12 nts, at least a 13 nts, at least a 14 nts, at least a 15 nts, at least a 16 nts, at least a 17 nts, at least a 18 nts, at least a 19 nts, at least a 20 nts, at least a 21 nts, at least a 22 nts, at least a 23 nts, at least a 24 nts, at least a 25 nts, at least a 30 nts, or at least a 35 nts of a target sequence in the FXN gene (such target sequence is followed or preceded by a PAM in the FXN gene but is not part of the gRNA). In embodiments, the “gRNA guide sequence” or “gRNA target sequence” may be least 10 nucleotides long, preferably 10-40 nts long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nts long), more preferably 17-30 nts long, more preferably 17-22 nucleotides long. In embodiments, the gRNA guide sequence is 10-40, 10-30, 12-30, 15-30, 18-30, or 10-22 nucleotides long. In embodiments, the PAM sequence is “NGG”, where “N” can be any nucleotide. In embodiments, the PAM sequence is “TTTN”, where “N” can be any nucleotide. In embodiments, the PAM sequence is “NNNNRYAC”, “NNNVRYAC” or “NNNNACAC”, where “N” can be any nucleotide, “V” is A, G or C, “R” is a purine (G or A), and “Y” is a pyrimidine (T or C). gRNAs may target any region of a target gene (e.g., FXN) which is immediately adjacent (contiguous, adjoining, in 5′ or 3′) to a PAM (e.g., NGG/TTTN/NNNNRYAC/NNNVRYAC/NNNNACAC, or CCN/NAAA/GTRYNNNN/GTRYBNNN/GTGTNNNN, for a PAM that would be located on the opposite strand) sequence. In embodiments, the gRNA of the present invention has a target sequence which is located (wholly or partly) in an exon (the gRNA guide sequence consists of the RNA sequence of the target (DNA) sequence which is located in an exon) but the cut is preferably in an intron. In embodiments, the gRNA of the present invention has a target sequence which is located in an intron (the gRNA guide sequence consists of the RNA sequence of the target (DNA) sequence which is located in an intron). In embodiments, the gRNA may target any region (sequence) which is followed (or preceded, depending on the CRISPR nuclease used) by a PAM in the FXN gene which may be used to restore or increase FXN expression level and/or activity.

The number of gRNAs administered to or expressed in a target cell in accordance with the methods of the present invention may be at least 1 gRNA, at least 2 gRNAs, at least 3 gRNAs at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, at least 16 gRNAs, at least 17 gRNAs, or at least 18 gRNAs. The number of gRNAs administered to or expressed in a cell may be between at least 1 gRNA and 15 gRNAs, 1 gRNA and least 10 gRNAs, 1 gRNA and 8 gRNAs, 1 gRNA and 6 gRNAs, 1 gRNA and 4 gRNAs, 1 gRNA and gRNAs, 2 gRNA and 5 gRNAs, or 2 gRNAs and 3 gRNAs.

Although a perfect match between the gRNA guide sequence and the DNA sequence on the targeted gene is preferred, a mismatch between a gRNA guide sequence and target sequence on the gene sequence of interest is also permitted as along as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. Preferably, the gRNA of the present invention comprises 7 mismatches, 6 mismatches, 5 mismatches, 4 mismatches, 3 mismatches, more preferably 2 mismatches, or less, and even more preferably no mismatch, with the corresponding gRNA target gene sequence (less the PAM). Preferably, the gRNA nucleic acid sequence is at least 90%, 91%, 92%, 93%, 94%, 95%, 96%, 97%, 98% and 99% identical to the gRNA target polynucleotide sequence in the gene of interest (e.g., FXN). Of course, the smaller the number of nucleotides in the gRNA guide sequence the smaller the number of mismatches tolerated. The binding affinity is thought to depend on the sum of matching gRNA-DNA combinations.

Any gRNA guide sequence can be selected in the target gene, as long as it allows introducing at the proper location, the desired modification(s) (e.g., spontaneous insertions/deletions or selected target modification(s) using one or more patch/donor sequence(s)). Accordingly, the gRNA guide sequence or target sequence of the present invention may be in coding or non-coding regions of the FXN gene (i.e., exons or introns, preferably intron 1). Of course the complementary strand of the sequence (e.g., reverse complement of SEQ ID NO: 4) may alternatively and equally be used to identify proper PAM and gRNA target/guide sequences.

### CRISPR Nucleases

The recombinant CRISPR nuclease that may be used in accordance with the present invention is i) derived from a naturally occurring Cas or related nuclease (e.g., Cpf1); and ii) has a nuclease activity to introduce a DSB in cellular DNA when in the presence of appropriate gRNA(s). Thus, as used herein, the term “CRISPR nuclease” refers to a recombinant protein which is derived from a naturally occurring nuclease which has nuclease activity and which functions with the gRNAs of the present invention to introduce DSBs in the targets of interest, e.g., the FXN gene. In embodiments, the CRISPR nuclease is CjCas9, SpCas9 or SaCas9. In embodiments, the CRISPR nuclease is Cpf1. In a further embodiment, the Cas protein is a dCas9 protein fused with a dimerization-dependant FoKI nuclease domain. Exemplary CRISPR nucleases that may be used in accordance with the present invention are provided in Table 1 above. A variant of Cas9 can be a Cas9 nuclease that is obtained by protein engineering or by random mutagenesis (i.e., is non-naturally occurring). Such Cas9 variants remain functional and may be obtained by mutations (deletions, insertions and/or substitutions) of the amino acid sequence of a naturally occurring Cas9, such as that of S. pyogenes.

CRISPR nucleases such as Cas9 nucleases cut 3-4bp upstream of the PAM sequence. CRISPR nucleases such as Cpf1 on the other hand, generate a 5′ overhang. The cut occurs 19 bp after the PAM on the targeted (+) strand and 23 bp on the opposite strand (62). Table 1 above provides the PAM sequence and cut site for exemplary CRISPR nucleases. There can be some off-target DSBs using wildtype Cas9. The degree of off-target effects depends on a number of factors, including: how closely homologous the off-target sites are compared to the on-target site, the specific site sequence, and the concentration of nuclease and guide RNA (gRNA). These considerations only matter if the PAM sequence is immediately adjacent to the nearly homologous target sites. The mere presence of additional PAM sequences should not be sufficient to generate off target DSBs; there needs to be extensive homology of the protospacer followed or preceded by PAM.

### Optimization of Codon Degeneracy

Because CRISPR nuclease proteins are (or are derived from) proteins normally expressed in bacteria, it may be advantageous to modify their nucleic acid sequences for optimal expression in eukaryotic cells (e.g., mammalian cells) when designing and preparing CRISPR nuclease recombinant proteins.

Accordingly, the following codon chart (Table 2) may be used, in a site-directed mutagenic scheme, to produce nucleic acids encoding the same or slightly different amino acid sequences of a given nucleic acid:

TABLE 2
Codons encoding the same amino acid

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUG AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

### Methods of Modifying Frataxin

The present invention provides a method of modifying within a cell, a frataxin (FXN) gene comprising a GAA repeat expansion. The method uses gRNAs in combination with a CRISPR nuclease and allow to introduce cuts (e.g., double stranded breaks with blunt ends or 5′ overhang) into DNA at specific sites. The cuts introduced in the gene FXN gene in accordance with the present invention allow to remove all or some of the endogenous sequence comprising the abnormal number of GAA trinucleotide repeats in intron 1 which leads to reduced FXN protein expression and causes FRDA.

Accordingly, in embodiments, methods of the present invention comprise introducing cuts within intron 1 of the FXN gene comprising an endogenous abnormal number of GAA trinucleotide repeats causing FRDA (e.g., 70 or more, 100 or more, 150 or more, 300 or more, 500 or more 800 or more or 1000 or more GAA repeats). In embodiments, the position of each cut is selected from cuts set forth in Table 3, 5, 6 or 7.

Although the entire intron 1 could be removed in accordance with the present invention, preferably, only a portion of the nucleic acid sequence of intron 1 is deleted on each side of the GAA repeats. In embodiments, a first cut is introduced within about 2000, preferably about 1000 and more preferably within about 550 nts from the first nucleotide of the GAA repeats (e.g., in 5′ or upstream of the beginning of the GAA repeats). Similarly, in embodiments, a second cut is introduced within about 2000, preferably about 1000 and more preferably within about 550 nts from the last nucleotide of the GAA repeats (e.g., in 3′ or downstream of the beginning of the GAA repeat sequence). In embodiments, the first and second cuts are made as close as possible from each end of the GAA repeats so as to remove the smallest number of nucleotides from intron 1 (e.g., within 200, within 150, within 124, within 100, within 75, within 50, within 30, within 35, or within 20 nucleotides or less from each end of the GAA repeat sequence).

Under certain conditions, gRNAs of the present invention may cut within the GAA repeat expansion, such that a portion of the GAA repeat expansion may be removed (i.e., a subset of the GAA repeats). For example, if a target sequence of a gRNA is sufficiently close to (or overlaps) the 5′ or 3′ end of the GAA repeat expansion, the cut introduced by the CRISPR nuclease may be within the GAA repeat expansion. As known in the art, CRISPR nuclease cuts in 5′ or 3′ of the PAM. The distance of the cut from the PAM depends on the CRISPR nuclease used. Under these conditions, introduction of cuts within the FXN gene followed by NHEJ may generate a modified FXN gene in which a portion of the GAA repeats remain. The presence of a small number of GAA repeats (e.g., less than 70) is known to not significantly affect FXN expression. Therefore modified FRDA cells in which some GAA repeats have been removed and some GAA repeats remain would nevertheless express FXN to a level above the base level of FXN expression in the unmodified FRDA cells.

Accordingly, in embodiments, the first cut of the first gRNA is within the GAA repeat expansion, preferably near the 5′ end of the GAA repeat expansion. In embodiments, the second cut of the second gRNA is within the GAA repeat expansion, preferably near the 3′ end of the GAA repeat expansion, i.e., downstream from the first cut. In embodiments, ligation of the first and second intron ends in accordance with methods of the present invention generates a modified FXN gene having 150 or fewer GAA repeats. Preferably, ligation of the first and second intron ends in accordance with methods of the present invention generates a modified FXN gene having 70 or fewer GAA repeats. In preferred embodiments, methods of the present invention allow removal of the entire GAA repeat expansion, i.e. all the GAA repeats, in intron 1 of the FXN gene of FRDA cells. Preferably, ligation of the first and second intron ends in accordance with methods of the present invention occurs by non-homologous end joining (NHEJ).

In embodiments, gRNAs and CRISPR nucleases which are used in accordance with the present invention allow removal at least 10, at least 50, at least 100, at least 200, at least 300, at least 500, at least 600, at least 700, at least 800 GAA repeats in the FXN gene of a cell. In embodiments, gRNAs and CRISPR nucleases which are used in accordance with the present invention allow removal of at least 50%, 60%, 70%, 80% or 90% of the GAA repeats in the FXN gene of a cell. Preferably, gRNAs and CRISPR nucleases of the present invention a portion of the GAA repeat extension in FRDA cells which is sufficient to increase FXN expression above the base level of FXN expression in the unmodified FRDA cells. Preferably, the complete GAA repeat expansion within an intron of the FXN gene.

gRNAs of the present invention are preferably between 17 and 20 nucleotides long. Non-limiting examples of gRNA target sequences are provided in Tables 5, 6 and 7. Thus, gRNAs having a target sequence corresponding to at least 17 consecutive nucleotides of intron 1 of the FXN gene or of a gRNA target sequence listed in Tables 5, 6 and 7 and genetic variants thereof, can be used in accordance with the present invention. Of course the target sequence should also be suitably positioned with respect to the PAM of the selected CRISPR nuclease.

In embodiments, gRNAs of the present invention comprise a target sequence which is set forth in Table 5, 6 or 7. In particular embodiments, the polynucleotide sequence removed on each side of the GAA repeat expansion in the FXN gene comprises (or consists of) polynucleotide sequences set forth in SEQ ID NOs: 100-126 and 158-167 (see also Tables 5, 6 and 7).

Although any suitable combinations of gRNAs may be used in accordance with the present invention, Table 3 below shows exemplary combination of gRNAs allowing to remove GAA trinucleotide repeats from intron 1 of the FXN gene.

TABLE 3
Sequences removed in intron 1 of FXN gene using Exemplary
combinations of gRNAs.

| | | | Total of nts |
|---|---|---|---|
| | # nts removed | #nts removed in | removed apart |
| gRNA | in 5′ of GAA | 3′ of GAA | from GAA |
| combination | repeats | repeats | repeats |
| AC1AC6 | 159 | 412 | 571 |
| AC2AC6 | 93 | 412 | 505 |
| AC3AC6 | 30 | 412 | 442 |
| C1C11 | 142 | 20 | 162 |
| C2C11 | 136 | 20 | 156 |
| C1C20 | 142 | 403 | 545 |
| C2C20 | 136 | 403 | 539 |
| C15C18 | 506 | 478 | 984 |
| C15C20 | 506 | 403 | 909 |
| C16C18 | 457 | 478 | 935 |
| C16C20 | 457 | 403 | 860 |
| Cj1Cj6 | 321 | 323 | 644 |
| Cj1Cj7 | 321 | 241 | 562 |
| Cj1Cj8 | 321 | 286 | 607 |
| Cj1Cj9 | 321 | 302 | 623 |
| Cj1Cj10 | 321 | 346 | 667 |
| Cj2Cj6 | 310 | 323 | 633 |
| Cj2Cj7 | 310 | 241 | 551 |
| Cj2Cj8 | 310 | 286 | 596 |
| Cj2Cj9 | 310 | 302 | 612 |
| Cj2Cj10 | 310 | 346 | 656 |
| Cj3Cj6 | 264 | 323 | 587 |
| Cj3Cj7 | 264 | 241 | 505 |
| Cj3Cj8 | 264 | 286 | 550 |
| Cj3Cj9 | 264 | 302 | 566 |
| Cj3Cj10 | 264 | 346 | 610 |
| Cj4Cj6 | 220 | 323 | 543 |
| Cj4Cj7 | 220 | 241 | 461 |
| Cj4Cj8 | 220 | 286 | 506 |
| Cj4Cj9 | 220 | 302 | 522 |
| Cj4Cj10 | 220 | 346 | 566 |
| Cj5Cj6 | 201 | 323 | 524 |
| Cj5Cj7 | 201 | 241 | 442 |
| Cj5Cj8 | 201 | 286 | 487 |
| Cj5Cj9 | 201 | 302 | 503 |
| Cj5Cj10 | 201 | 346 | 547 |

In embodiments, the first cut in the FXN gene is within about 625 nucleotides from the end of the GAA repeats (i.e., upstream or 5′ from the first nucleotide of the GAA repeat sequence). In embodiments, the first cut in the FXN gene is within about 519 nucleotides from the end of the GAA repeats (i.e., upstream or 5′ from the first nucleotide of the GAA repeat sequence). In embodiments, the first cut in the FXN gene is within about 506 nucleotides from the end of the GAA repeats (i.e., upstream or 5′ from the first nucleotide of the GAA repeat sequence). In embodiments, the first cut in the FXN gene is within about 457 nucleotides from the end of the GAA repeats (i.e., upstream or 5′ from the first nucleotide of the GAA repeat sequence). In embodiments, the first cut in the FXN gene is within about 178 nucleotides from the end of the GAA repeats (i.e., upstream or 5′ from the first nucleotide of the GAA repeat sequence). In embodiments, the first cut in the FXN gene is within about 159 nucleotides from the end of the GAA repeats (i.e., upstream or 5′ from the first nucleotide of the GAA repeat sequence). In embodiments, the first cut in the FXN gene is within about 142 nucleotides from the end of the GAA repeats (i.e., upstream or 5′ from the first nucleotide of the GAA repeat sequence). In embodiments, the first cut in the FXN gene is within about 136 nucleotides from the end of the GAA repeats (i.e., upstream or 5′ from the first nucleotide of the GAA repeat sequence). In embodiments, the first cut in the FXN gene is within about 93 nucleotides from the end of the GAA repeats (i.e., upstream or 5′ from the first nucleotide of the GAA repeat sequence). In embodiments, the first cut in the FXN gene is within about 81 nucleotides from the end of the GAA repeats (i.e., upstream or 5′ from the first nucleotide of the GAA repeat sequence). In embodiments, the first cut in the FXN gene is within about 76 nucleotides from the end of the GAA repeats (i.e., upstream or 5′ from the first nucleotide of the GAA repeat sequence). In embodiments, the first cut in the FXN gene is within about 58 nucleotides from the end of the GAA repeats (i.e., upstream or 5′ from the first nucleotide of the GAA repeat sequence). In embodiments, the first cut in the FXN gene is within about 30 nucleotides from the end of the GAA repeats (i.e., upstream or 5′ from the first nucleotide of the GAA repeat sequence).

In embodiments, the second cut in the FXN gene is within about 597 nucleotides from the end of the GAA repeats (i.e., downstream or 3′ from the last nucleotide of the GAA repeat sequence). In embodiments, the second cut in the FXN gene is within about 493 nucleotides from the end of the GAA repeats (i.e., downstream or 3′ from the last nucleotide of the GAA repeat sequence). In embodiments, the second cut in the FXN gene is within about 478 nucleotides from the end of the GAA repeats (i.e., downstream or 3′ from the last nucleotide of the GAA repeat sequence). In embodiments, the second cut in the FXN gene is within about 412 nucleotides from the end of the GAA repeats (i.e., downstream or 3′ from the last nucleotide of the GAA repeat sequence). In embodiments, the second cut in the FXN gene is within about 403 nucleotides from the end of the GAA repeats (i.e., downstream or 3′ from the last nucleotide of the GAA repeat sequence). In embodiments, the second cut in the FXN gene is within about 126 nucleotides from the end of the GAA repeats (i.e., downstream or 3′ from the last nucleotide of the GAA repeat sequence). In embodiments, the second cut in the FXN gene is within about 114 nucleotides from the end of the GAA repeats (i.e., downstream or 3′ from the last nucleotide of the GAA repeat sequence). In embodiments, the second cut in the FXN gene is within about 86 nucleotides from the end of the GAA repeats (i.e., downstream or 3′ from the last nucleotide of the GAA repeat sequence). In embodiments, the second cut in the FXN gene is within about 50 nucleotides from the end of the GAA repeats (i.e., downstream or 3′ from the last nucleotide of the GAA repeat sequence). In embodiments, the second cut in the FXN gene is within about 49 nucleotides from the end of the GAA repeats (i.e., downstream or 3′ from the last nucleotide of the GAA repeat sequence). In embodiments, the second cut in the FXN gene is within about 22 nucleotides from the end of the GAA repeats (i.e., downstream or 3′ from the last nucleotide of the GAA repeat sequence). In embodiments, the second cut in the FXN gene is within about 20 nucleotides from the end of the GAA repeats (i.e., downstream or 3′ from the last nucleotide of the GAA repeat sequence).

In embodiments, gRNAs of the present invention have a target sequence adjacent to a NGG PAM nucleotide sequence in intron 1 of the FXN gene corresponding to the following nucleotide positions: a) nts 6579-6577; (b) nts 6592-6594; (c) nts 6543-6541; (d) nts 6670-6672; (e) nts 6645-6643; (f) nts 6647-6649; (g) nts 6761-6759; (h) nts 6832-6834; (i) nts 6888-6886; (j) nts 6853-6851; (k) nts 6766-6768; (I) nts 6872-6874; (m) nts 6202-6200; (n) nts 6103-6105; (o) nts 6221-6223; (p) nts 6264-6262; (q) nts 7232-7230; (r) nts 7324-7326; (s) nts 7336-7334; or (t) nts 7142-7141. In embodiments, gRNAs of the present invention have a target sequence adjacent to a NNGRRT PAM nucleotide sequence in intron 1 of the FXN gene corresponding to the following nucleotide positions: a) nts 6569-6574; (b) nts 6635-6640; (c) nts 6691-6686; (d) nts 6789-6784; (e) nts 7078-7073; or (f) nts 7158-7163. All nucleotides positions on the frataxin gene described herein are with respect to nucleotides comprised in intron 1 of the frataxin gene set forth in GenBank NG_00845 (SEQ ID NO: 4).

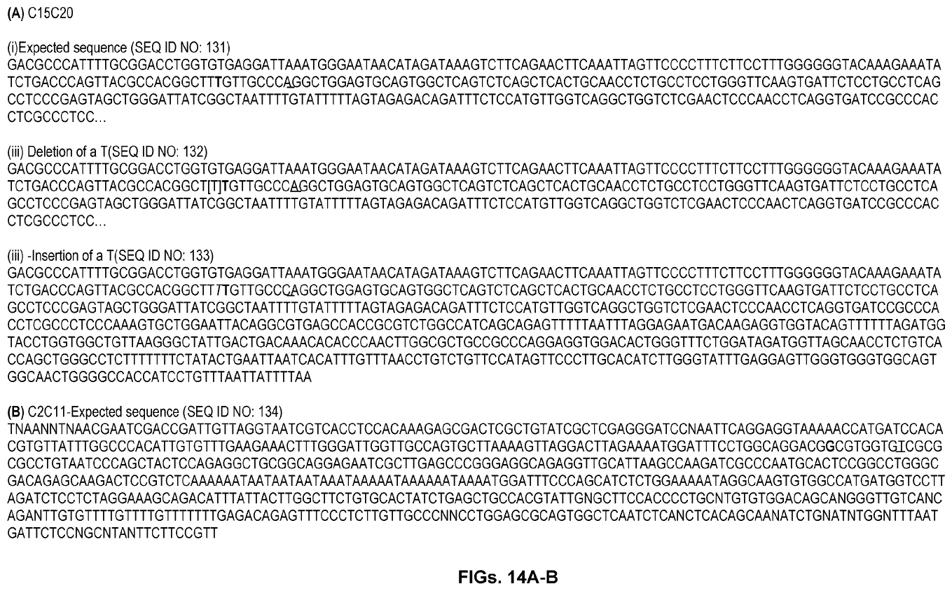

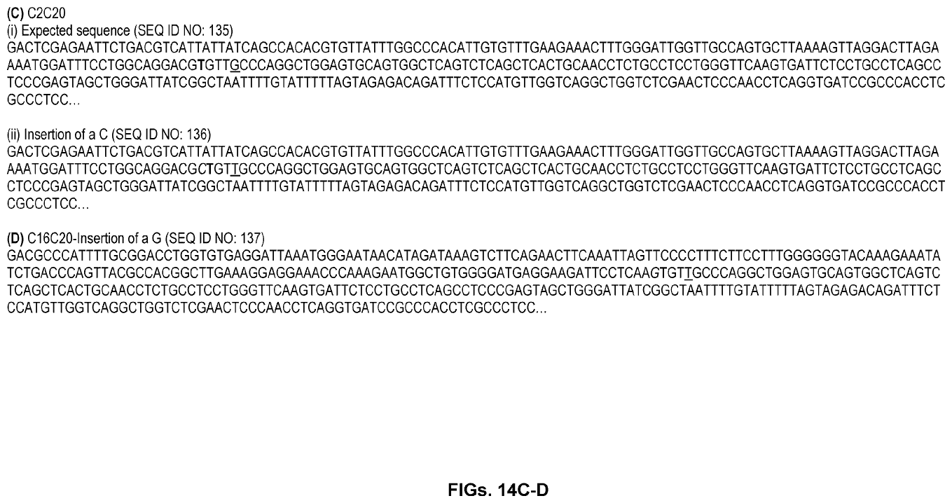

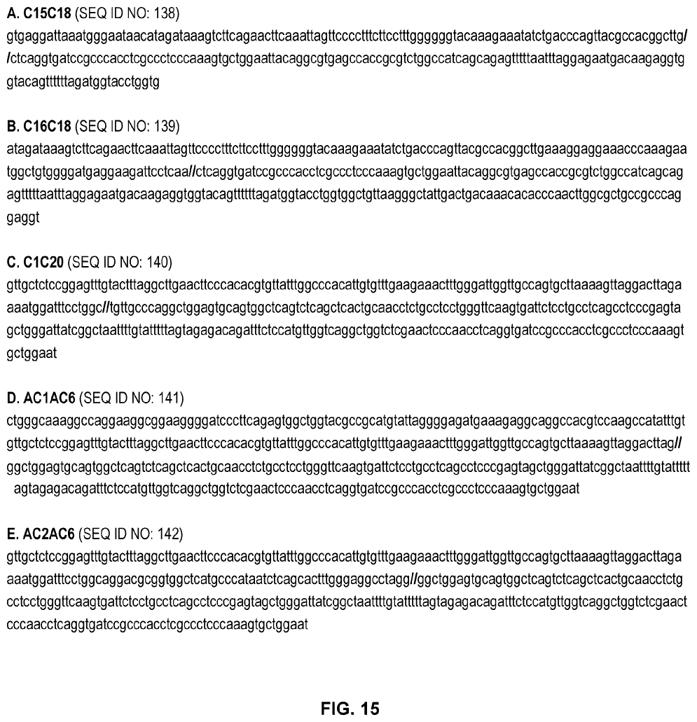

In embodiments, methods of the present invention generate a modified FXN gene (in which the GAA trinucleotide repeats have been removed) comprising in intron 1 a modified polynucleotide sequence as set forth in FIG. 14 or 15 (any one of SEQ ID NOs: 131-142) or any one of SEQ ID NOs: 171-195.

As any other nucleic acid gene sequence, endogenous sequence variations in intron 1 of the FXN gene exist between individuals (allelic/genetic variants). Such variant nucleic acid sequences are retrievable from well-known databases and websites such as NCBI, Ensembl, Vega, OMIM and others (e.g., ClinVar and dbVar databases and NCBI variation viewer. See for example www.ncbi.nlm.nih.gov/gene/2395#variation). Accordingly, gRNAs of the present invention target any naturally occurring genetic variants of the FXN gene which can be found in a population. Thus, as used herein, the term “frataxin (FXN) gene” encompasses any frataxin gene found within a cell and includes variants (e.g., allelic/genetic variants) of the frataxin gene polynucleotide sequence in SEQ ID NO: 4.

As indicated above, nucleic acids encoding gRNAs and nucleases (e.g., Cas9 or Cpf1) of the present invention may be delivered into cells using one or more various vectors such as viral vectors. Accordingly, preferably, the above-mentioned vector is a viral vector for introducing the gRNA and/or nuclease of the present invention in a target cell. Non-limiting examples of viral vectors include retrovirus, lentivirus, Herpes virus, adenovirus or Adeno Associated Virus, as well known in the art.

The modified AAV vector preferably targets one or more cell types affected in FRDA subjects. In an embodiment, the cell type is a muscle cell, in a further embodiment, a myoblast. Accordingly, the modified AAV vector may have enhanced cardiac, skeletal muscle, neuronal, liver, and/or pancreatic tissue (Langerhans cells) tropism. The modified AAV vector may be capable of delivering and expressing the at least one gRNA and nuclease of the present invention in the cell of a mammal. For example, the modified AAV vector may be an AAV-SASTG vector (Piacentino et al. (2012) Human Gene Therapy 23:635-646). The modified AAV vector may deliver gRNAs and nucleases to neurons, skeletal and cardiac muscle, and/or pancreas (Langerhans cells) in vivo. The modified AAV vector may be based on one or more of several capsid types, including AAVI, AAV2, AAVS, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery. In an embodiment, the modified AAV vector is a AAV-DJ. In an embodiment, the modified AAV vector is a AAV-DJ8 vector. In an embodiment, the modified AAV vector is a AAV2-DJ8 vector. In an embodiment, the modified AAV vector is a AAV-PHP.B vector. In an embodiment, the modified AAV vector is a AAV-PHP.B, AAV-9 or AAV-DJ8 (PHP.B: PMID: 26829320, PMID: 27867348; AAV DJ-8: www.cellbiolabs.com/news/aav-helper-free-expression-systems-aav-dj-aav-dj8, http://www.cellbiolabs.com/aav-expression-and-packaging; www.cellbiolabs.com/scaav-dj8-helper-free-complete-expression-systems; and AAV9: PMID: 27637390, PMID: 16713360).

In another aspect, the present invention provides a composition (e.g., a pharmaceutical composition) comprising the above-mentioned gRNA and/or CRISPR nuclease (e.g., Cas9), or nucleic acid(s) encoding same or vector(s) comprising such nucleic acid(s). In an embodiment, the composition further comprises one or more pharmaceutically acceptable or biologically acceptable carriers, excipients, and/or diluents.

As used herein, “pharmaceutically acceptable” refers to materials characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio. “Biologically acceptable” refers to materials characterized by the absence of (or limited) toxic or adverse biological effects in biological systems, e.g., in vitro or in vivo, i.e., compatible for use with living cells without excessive toxicity.

The present invention further provides a kit or package comprising at least one container means having disposed therein at least one of the above-mentioned gRNAs, nucleases, vectors, cells, targeting systems, combinations and/or compositions. In an embodiment, the kit or package further comprises instructions for removing the GAA repeat expansion in the FXN gene in a cell or for treatment of FRDA in a subject.

### Definitions

In order to provide clear and consistent understanding of the terms in the instant application, the following definitions are provided.

The articles “a,” “an” and “the” are used herein to refer to one or to more than one (i.e., to at least one) of the grammatical object of the article.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps and are used interchangeably with, the phrases “including but not limited to” and “comprising but not limited to”.

For the recitation of numeric ranges herein, each intervening number there between with the same degree of precision is explicitly contemplated. For example, for the range of 18-20, the numbers 18, 19 and 20 are explicitly contemplated, and for the range 6.0-7.0, the number 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

Practice of the methods, as well as preparation and use of the products and compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Coding sequence” or “encoding nucleic acid” as used herein means the nucleic acids (RNA or DNA molecule) that comprise a nucleotide sequence which encodes a protein or gRNA. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

### Sequence Similarity

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “substantially homologous” to another sequence if the two sequences are substantially identical and the functional activity of the sequences is conserved (as used herein, the term “homologous” does not infer evolutionary relatedness, but rather refers to substantial sequence identity, and thus is interchangeable with the terms “identity”/“identical”). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. For the sake of brevity, the units (e.g., 66, 67 . . . 81, 82, . . . 91, 92%, . . . ) have not systematically been recited but are considered, nevertheless, within the scope of the present invention.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 96%, at least 98% or at least 99%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman (Pearson and Lipman 1988), and the computerized implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al. (Altschul et al. 1990), using the published default settings. Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (Ausubel 2010). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (Ausubel 2010). Hybridization conditions may be modified in accordance with known methods depending on the sequence of interest (Tijssen 1993). Generally, stringent conditions are selected to be about 5° C. lower than the thermal melting point for the specific sequence at a defined ionic strength and pH.

“Promoter” as used herein means a synthetic or naturally-derived nucleic acid molecule which is capable of conferring, modulating or controlling (e.g., activating, enhancing and/or repressing) expression of a nucleic acid in a cell. A promoter may comprise one or more specific transcriptional regulatory sequences to further enhance or repress expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV, CMV IE promoter, SV40 early promoter or SV40 late promoter and the CMV IE promoter. In embodiments, the U6, Cbh, CMV and/or H1m promotor is used to express one or more gRNAs in a cell.

A “WPRE sequence” refers to the Woodchuck Hepatitis Virus (WHP) Posttranscriptional Regulatory Element (WPRE) which is a DNA sequence that, when transcribed, creates a tertiary structure which may enhance expression. The sequence is commonly used in molecular biology to increase expression of genes delivered by viral vectors. WPRE is a tripartite regulatory element with gamma, alpha, and beta components. The full tripartite sequence has 100% homology with base pairs 1093 to 1684 (SEQ ID NO: 170 or 196) of the Woodchuck hepatitis B virus (WHV8) genome. When used in the 3′ untranslated region (UTR) of a mammalian expression cassette, it can significantly increase mRNA stability and protein yield.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self- replicating extrachromosomal vector, and preferably, is a DNA plasmid. For example, the vector may comprise nucleic acid sequence(s) that/which encode(s) a gRNA, a donor (or patch) nucleic acid, and/or a CRISPR nuclease (e.g., Cas9 or Cpf1) of the present invention. A vector for expressing one or more gRNA will comprise a “DNA” sequence of the gRNA.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In an embodiment, the subject or patient may suffer from FRDA and has an abnormal GAA trinucleotide repeat expansion. The subject or patient may be undergoing other forms of treatment.

The present invention is illustrated in further details by the following non-limiting examples.

### EXAMPLE 1

### Materials and Methods

DNA constructs. Plasmids used in this study included the following: px330 as px330-U6-Chimeric_BB-CBh-hSpCas9 (Addgene plasmid #42230)(43), pxGFP or pxPuro as pSpCas9(BB)-2A-GFP or Puro (Addgene plasmids #48138/48139)(44) and px601 as px601-AAV-CMV::NLS-SaCas9-NLS-3xHA-bGHpA;U6::Bsal- gRNA (Addgene plasmid #61591) (31) which were provided by Feng Zhang (Department of Genetics, Harvard Medical School, Boston, Mass., USA). Plasmids used also included the pRGEN-CMV-CjCas9 plasmid (Addgene #89752) the pU6-Cj- gRNA plasmid (Addgene #89753), which were provided by Seokjoon Kim (ToolGen, Geumcheon-gu, Seoul, South Korea). Others plasmids included a recombinant AAV vector backbone modified from the pAAV_TALE-TF (VP64)-BB_V3 (Addgene#42581), provide by Feng Zhang (Department of Genetics, Harvard Medical School, Boston, Mass., USA). Oligonucleotides coding for guide RNAs were synthetized by Integrated DNA Technologies (IDT inc., Coralville, Iowa) and cloned into Bbsl (or Bsal for px601) restriction sites according to Zhang's guidelines (https://www.addgene.org/static/cms/filer_public/e6/5a/e65a9ef8-c8ac-4f88-98da-3b7d7960394c/zhang-lab-general-cloning-protocol.pdf). All DNA constructs were sent for sequencing using the primer U6F (5′-GTCGGAACAGGAGAGCGCACGAGGGAG, SEQ ID NO: 5) or the H1F primer (5′-TGTCGCTATGTGTTCTGGGA, SEQ ID NO: 211) to the Genomic sequencing and genotyping platform of the CHU de Québec (Quebec City, QC, Canada).

When needed, PCR amplicons from plasmidic or genomic DNA were cloned into the linearized cloning vector pMiniT™ (NEB, 1pwisch, Mass.) and sequenced using the manufacturer provided forward and reverse primers.

Modifications of the original px601 vector were performed as follows. The CMV promoter (577 bp, located between Xhol and Agel sites) was replaced by short versions of 212 or 259 bp amplified from the pscAAV-GFP plasmid from John T. Gray (Addgene plasmid #32396) (45) and according to previous experimentations published by Senis and al. (32). The px601 polyA sequence (204 bp, included in the sequence between EcoRl and Kpnl sites) was replaced by a short version of 60 bp (32) cloned as a gBLOCK (IDT inc., Coralville, Iowa) while preserving the Kpnl restriction site. A sequence comprising the H1 minimal promoter (H1m), a selected cloned oligonucleotide gRNA-coding and the SaCas9 tracrRNA was amplified from the home made pGL3 H1m/BbsI/SaCas9 and cloned into the Kpnl site of the newly prepared px601 vector. Finally, and if not previously included in the plasmid, the second oligonucleotide coding for a gRNA was cloned into the Bsal site following the U6 promoter.

Modifications of the original pAAV_TALE-TF (VP64)-BB_V3 were performed as follows. A fragment containing the CjCas9 gene (amplified from the pRGEN-CMV-CjCas9 plasmid under the control of a CBh promoter, followed by a SV40 late poly A and a WPRE sequence and two gRNAs expressed from either the human U6 promoter or a minimal H1 promoter was clone into a gel purified Xbal/Notl digested pAAV plasmid.

All PCR amplifications were performed using the Phusion™ High Fidelity polymerase (Thermo Fisher Scientific inc., Waltham, Mass.). All cloning were performed using the In-Fusion HD™ cloning kit (Clontech Laboratories inc., Mountain View, Calif.). Plasmid design and sequencing analysis were done using the CLC main workbench software version 7.6 (CLC bio/Qiagen inc., Hilden, Germany).

Mouse cells and animal model. Mouse fibroblasts derived from the YG8R and YG8sR mouse models were obtained from Dr. MA Pook (Brunel University, London, UK). Characterization of these mice by Pook's group revealed that the YG8R fibroblasts carried two tandem copies of the human FXN gene with about 82 and 190 GAA trinucleotide sequence repeats (25) while the YG8sR has 190 GAA repeats. The Y47R cell line, which has been produced and isolated the same way as the YG8R cell line, contains however a single copy of the wild-type human FXN transgene, with about 9 GAA trinucleotide repeats (25). The mouse fibroblasts were cultured at 37° C., 5% CO₂ in high glucose DMEM (Wisent inc., St-Bruno, QC, Canada) supplemented with 10% fetal bovine serum (GE healthcare Life Sciences inc., Mississauga, ON, Canada), 1 mM sodium pyruvate, 1 mM L-glutamine and 1X non-essential amino acids (Wisent inc.).

The mouse model YG8R (Fxntm1Mkn /Tg (FXN)YG8Pook/J)(25) homozygous for the Fxntm1Mkn (FXN) targeted allele and hemizygous for the Tg (FXN)YG8Pook (FXN, human) transgene was purchased from the Jackson Laboratory (stock number 012253, Bar Harbor, Minn).

Transfections and clonal expansion. Mouse YG8R or YG8sR fibroblasts or human 283T or FRDA cells were seeded and transfected at 70-80% confluence with DNA using Lipofectamine™ 2000 (Life Technologies inc., Carlsbad, Calif.) according to the manufacturer's instructions. Cells were harvested 48 hours later for DNA, RNA and protein analysis. For clonal expansion, puromycin (0.75 pg/ml) was added 24 h post-transfection and 48 h later, remaining cells were seeded in 96-well plates at 0.75 cell/well and expanded.

Five hundred thousand (5×10⁵) normal or FRDA fibroblasts (YG8R or YG8sR, passages≤10) were nucleofected using the Amaxa™ system and program P-022 for normal human dermal adult fibroblasts (VPD-1001, Lonza inc., Walkersville, Md.). Cells were harvested 72 hours later for genomic DNA or RNA transcriptional analysis. When needed, fluorescence from transfected cells was visualized using a Zeiss Axiovert 100™-Inverted microscope (Zeiss inc., Oberkochen, Germany).

For experiments using human FRDA fibroblasts (Example 8), cells (#GM04078 and #GM03665) were purchased from the Coriell Institute (Boston, Mass.). One million (1×10⁶) cells (passages 10) were nucleofected using the Amaxa™ system as described above. Plasmids expressing the SpCas9 and C2C20 or C15C20 combinations or a ribonucleoproteic complex of 2.5 uM of SpCas9 protein (Feldan Therapeutics, Quebec, Canada) and 150 pmol of each gRNA in vitro transcribed (HiScribe™ T7 RNA high yield RNA synthesis kit #E2040S, New England Biolabs inc.) from DNA templates. Cells were harvested 72 hours later for genomic DNA. A primer-based assay using the F9 (5′- TCCCGGTTGCATTTACACTG, SEQ ID NO: 9) and R3 (5′-AGGGGGAGCTTAGGGTCAAT, SEQ ID NO: 11) primer set was used to amplify the corrected, GAA deleted, DNA molecules. The FRDA fibroblasts were cultured at 37° C., 5% CO2 in high glucose DMEM (Wisent inc., St-Bruno, QC, Canada) supplemented with 10% fetal bovine serum (GE healthcare Life Sciences inc., Mississauga, ON, Canada), 1 mM sodium pyruvate, 1 mM L-glutamine and 1X non-essential amino acids (Wisent inc.).

In vivo DNA electrotransfer. The electrotransfer was performed in the Tibialis anterior (TA) of adult YG8LR mice as previously described (46). Briefly, 40 μg of DNA consisting of a mixture of two pxGFP plasmids (encoding for SpCas9 and two gRNAs) were electroporated into the TA muscle of YG8LR mice. The latter were euthanized 1 month later, TAs were collected and genomic DNA was extracted immediately or the TA was embedded in OTC and snap-frozen in liquid nitrogen. PCR amplification was performed to detect deletions, according to the gRNA pair used. All experiments involving animals were approved by the animal care committee of the Centre Hospitalier Universataire de Québec-Université Laval (CHUQ-Université Laval).

AAV production and infection. Viruses were produced with the Plateforme d'outils moléculaires at Centre de recherche Institut Universitaire en Santé Mentale à Québec. 7.5×10¹¹ vector genomes of each AAV-Cas9 and AAV- gRNA C2C20 PHB.P-serotyped viruses were co-injected intravenously in month-old YG8sR. One month later, mice were euthanized and organs were collected (brain, medulla, spinal cord, dorsal root ganglia, liver, heart, Tibialis anterior and pancreas) and genomic DNA was extracted. A PCR was performed to detect the viruses in various samples using the Cas9 and the RSV primers. Digital droplet PCR (ddPCR) analysis of genomic DNA was performed using the following primers and probes to detect the non-edited molecules (Fw: GATTGGTTGCCAGTGCTTAAA, SEQ ID NO: 34; Rev: TCAGGTGATCCACCTTCCTA, SEQ ID NO: 35; Probe:5′-(HEX)-TGCCCATAATCTCA-(IABkFQ)-3′, SEQ ID NO: 36, HEX as the reporter and IOWA black FQ™ (IABkFQ) as the quencher) and edited molecules (Fw:GATTGGTTGCCAGTGCTTAAA, SEQ ID NO: 34; Rev:GTTGCAGTGAGCTGAGACT, SEQ ID NO: 37, Probe: 5′-(FAM)-AGTGCAGTGGCT-(IABkFQ)-3′ SEQ ID NO: 38, FAM as the reporter and IABkFQ as the quencher). Genomic DNA was digested using HindIII within the ddPCR pre-mix and droplets were generated using the droplet generator (Bio-Rad). Then, molecules were amplified as follows: 1 cycle at 95° C. 10 minutes then 40 cycles of 95° C. 30 seconds and 57° C. 45 seconds. Droplets were read using the droplet reader (Bio-Rad). Data analysis was performed using the Quantasoft™ software.

Genomic DNA analysis. Cells or tissues (TA) were harvested, resuspended in lysis buffer (50 mM EDTA pH 8.0, 10% Sarcosyl, 0.5 mg/ml Proteinase K) and genomic DNA was extracted using a standard phenol/chloroform and ethanol precipitation method. The polymerase chain reaction was done using primer sequences provided in Table 4. The conditions for PCR reactions, using the Phusion™ High Fidelity polymerase (Thermo Fisher Scientific inc., Waltham, Mass.) were: 35 cycles, denaturation at 98° C. for 10 sec, annealing at 60° C. for 10 sec, elongation at 72° C. for 90 sec. PCR products were visualized on agarose gel and if needed, submitted to the Surveyor Assay (Integrated DNA Technology inc., Coralville, Iowa) according to the manufacturer's instructions.

TABLE 4
Primers used in this study

| Primer name | Sequence 5′-3′ | Species |
|---|---|---|
| F1 | AAGAATGGCTGTGGGGATGA | Human |
| F2 | GTGGAAGCCCAATACGTGGC | Human |
| F3 | GCTTTCCTGGAACGAGGTGA | Human |
| F4 | GGATTTCCCAGCATCTCTGG | Human |
| F9 | TCCCGGTTGCATTTACACTG | Human |
| F10 | GGGTTGTCAGCAGAGTTGTG | Human |
| R3 | AGGGGGAGCTTAGGGTCAAT | Human |
| R9 | TGGCATCTTCAAGACCCTCA | Human |
| R10 | GGAGAAAAGGGTGGGGAAGA | Human |
| FXN exons 2/3 F | AAGCCATACACGTTTGAGGACTA | Human |
| FXN exons 2/3 R | TTGGCGTCTGCTTGTTGATCA | Human |
| FXN 5′UTR/exon1 F | GGCGGAGCGGGCGGCAGAC | Human |
| FXN 5′UTR/exon1 R | GGGGCGTGCAGGTCGCATCG | Human |
| hFXN exon 2 F | CCAACGTGGCCTCAACCAGAT | Human |
| hFXN exon 2 R | GGGTGGCCCAAAGTTCCAGAT | Human |
| mFXN exon 2 F | CATTTGAACCTCCACTACCTCCAGAT | Mouse |
| mFXN exon 2 R | TGTCCAATGTCCCCAAGTTCCTC | Mouse |
| hFXN promoter F | GTTGCAGTAAGCCAGGACCAC | Human |
| hFXN promoter R | GATCCACCCGCCTCATTTATTTG | Human |
| mFXN promoter F | GAGGCCATATCCCAGAAGAAAACT | Human |
| mFXN promoter R | CAGGCAGCATGAATGGAGGAG | Mouse |
| HPRT1 F | CAGGACTGAAAGACTTGCTCGAGAT | Mouse |
| HPRT1 R | CAGCAGGTCAGCAAAGAACTTATAGC | Mouse |
| GAPDH F | GGCTGCCCAGAACATCATCCCT | Mouse |
| GAPDH R | ATGCCTGCTTCACCACCTTCTTG | Mouse |
| Cas9 (fw) | AGATGATCGCCAAGAGCGAG | Humanized S.p |
| Cas9 (rev) | ATCCCCAGCAGCTCTTTCAC | Humanized S.p |
| RSV (fw) | TGCGGAATTCAGTGGTTCGT | RSV |
| RSV (rev) | AGCTACAACAAGGCAAGGCT | RSV |

Copy number analysis. Oligoprimer pairs were designed by GeneTool™ 2.0 software (Biotools inc, Edmonton, AB, CA) and their specificity was verified by blast in the GenBank database. The synthesis was performed by IDT (Integrated DNA Technology inc., Coralville, Iowa, USA) (Table 4).

40 ng of genomic DNA was used to perform fluorescent-based Realtime PCR quantification using the LightCycler 480 (Roche Diagnostics inc., Mannheim, Del.). Reagent LightCycler 480 SYBRGreen I Master (Roche Diagnostics inc., Indianapolis, Ind., USA) was used as described by the manufacturer. The conditions for PCR reactions were: 45 cycles, denaturation at 98° C. for 10 sec, annealing at 62° C. for 10 sec, elongation at 72° C. for 14 sec and reading for 5 sec. A melting curve was performed to assess non-specific signal. Relative quantity was calculated using the delta Ct method (47). Quantitative Real-Time PCR measurements were performed by the CHU de Québec Research Center (CHUL) Gene Expression Platform, Quebec, Canada and were compliant with MIQE guidelines (48, 49).

RNA analysis. Cells were harvested, resuspended in Trizol™ and RNA was isolated. Total RNA was measured using a NanoDrop ND-1000™ Spectrophotometer (NanoDrop Technologies inc., Wilmington, Del.) and total RNA quality was assayed on an Agilent BioAnalyzer 2100 (Agilent Technologies inc., Santa Clara, Calif.).

First-strand cDNA synthesis was done using 500 ng of isolated RNA in a reaction containing 200 U of Superscript III™ Rnase H-RT (Invitrogen Life Technologies inc., Burlington, ON, CA), 300 ng of oligo-dT18, 50 ng of random hexamers, 50 mM Tris-HCl pH 8.3, 75 mM KCl, 3 mM MgCl₂, 500 μM deoxynucleotides triphosphate, 5 mM dithiothreitol, and 40 U of Protector RNase inhibitor (Roche Diagnostics inc., Indianapolis, Ind.) in a final volume of 50 μl. Reaction was incubated at 25° C. for 10 min, then at 50° C. for 1 h and PCR purification kit (Qiagen inc., Hilden, Del.) was used to purify cDNA.

cDNA corresponding to 20 ng of total RNA was used to perform fluorescent-based Realtime PCR quantification using the LightCycler 480 (Roche Diagnostics inc., Mannheim, DE). Reagent LightCycler 480 SYBRGreen™ I Master (Roche Diagnostics inc., Indianapolis, Ind.) was used as described by the manufacturer with 2% DMSO. The conditions for PCR reactions were: 45 cycles, denaturation at 95° C. for 10 secs, annealing at 58° C. for 10 secs, elongation at 72° C. for 14 secs and then 74° C. for 5 sec (reading) using primers described in Table 4. A melting curve was performed to assess non-specific signal. Calculation of the number of copies of each mRNA was performed using second derivative method and a standard curve of Cp versus logarithm of the quantity (50). The standard curve was established using known amounts of purified PCR products (10, 102, 103, 104, 105 and 106 copies) and a LightCycler 480 v1.5 program provided by the manufacturer (Roche Diagnostics inc., Mannheim, Del.). The CHU de Québec Research Center (CR-CHUQ) Gene Expression Platform, Quebec, Canada, performed quantitative real-time PCR measurements.

Protein analysis. Cells were harvested and resuspended in lysis buffer (137 mM NaCl, 50 mM Tris-HCl pH8 and 0.1% Triton ^{X}100™) supplemented with 1X protease inhibitor cocktail (Roche Diagnostics Canada inc., Mississauga, ON, Canada). Protein extracts were loaded onto 12.5% SDS-PAGE and wet transfer was performed onto PVDF membrane. The latter was blotted using primary anti-FXN (ab110328, Abcam inc., Cambridge, UK or sc-25820, Santa Cruz Biotechnologies inc., Santa Cruz, Calif.), anti-HA (H-3663) anti-FLAG M2 (F-1804) and β-actin (A-1978) from Sigma-Aldrich inc. (St-Louis, Mo.) antibodies. Mouse and rabbit secondary antibodies were purchased from Jackson ImmunoResearch inc. (West Grove, Pa.).

### EXAMPLE 2

### Identification of gRNA Pairs Targeting Sequences Upstream and Downstream GAA TRINUCLEOTIDE Repeats

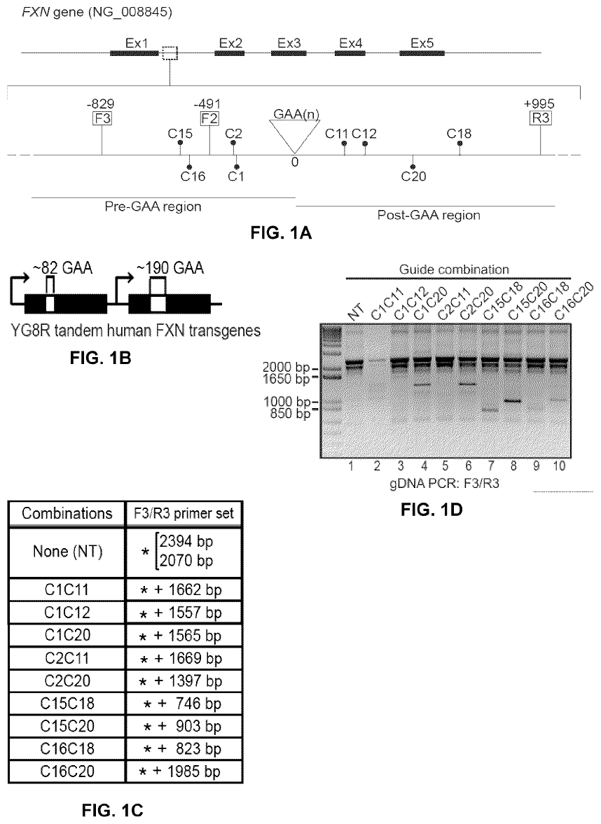

gRNAs targeting sequences located upstream (5′) and downstream (3′) of the GAA trinucleotide repeats in intron 1 of the FXN gene (NG_008845) were designed. Sequences adjacent to the S. pyogenes NGG PAM were first identified (FIG. 1A and Table 5) and 20 nts oligonucleotides targeting sequences located 5′ of the PAMs were prepared and cloned in an expression vector (px330, and/or pxPuro and/or pxGFP, Addgene; see Example 1) under the control of a RNA polymerase (pol) III U6 promoter. Vectors also encoded the SpCas9 protein under the control of a RNA pol II promoter (CBh).

The rescued YG8 (YG8R) mouse model is model system to study FRDA (24-26) which has been known for many years. The YG8R mouse genome contains 2 null mouse FXN genes but also 2 copies in tandem, of a FXN transgene obtained from an FRDA patient. These human transgenes contain respectively 82 and 190 GAA repeats in intron 1 and thus a reduced amount of human FXN is produced leading to the development of FRDA symptoms in mouse.

Therefore, plasmids were first transfected in mouse YG8R fibroblasts. These cells contain two human FRDA FXN transgenes in tandem comprising about 82 and 190 GAA repeats respectively (29) (FIG. 1B). PCR amplification of the polynucleotide sequence comprising GAA repeats using the F3/R3 primer set (FIG. 1A, see Example 1 for primer sequences) from genomic DNA (gDNA) of YG8R cells revealed the amplification of two bands at 2070 and 2394 bp (FIG. 1D, lane 1). Each band represents one of the two FXN transgenes (uncut form). Different pairs of gRNAs (one targeting the pre-GAA region and the other the post-GAA region) were tested and deletion efficiency was assessed by PCR using the F3/R3 primer set (FIGS. 1C, D). Effective sequence deletion between two targeted sequences on the FXN gene generates smaller PCR amplicons and allows the visualization of an additional smaller band (FIGS. 1C, D).

TABLE 5
Pre- and post-GAA repeat target sequences for S. pyogenes Cas9.
Position of first nucleotide of GAA repeats: 6725 and of last nucleotide of GAA
repeats: 6742 of FXN gene sequence set forth in SEQ ID NO: 4 (NG_008845).

| | | | | gRNA | | | | Distance | | |
|---|---|---|---|---|---|---|---|---|---|---|
| | | | | target | | | | of cut | | |
| | gRNA | | | sequence | | | | from | Sequence | Pre- |
| | sequence | | gRNA target | gene | | PAM gene | Cut site | first or | removed | or |
| | (SEQ ID | | sequence (5′-3′) | position | | position | gene | last GAA | (SEQ | post- |
| ID | NO.) | Strand | (SEQ ID NO.) | (5′-3′) | | (5′-3′) | position | repeat | ID NO.) | GAA |
| C1 | SEQ ID | Antisense | ATGAGCCACCGCGTCCTGCC | 6599-6580 | PAM | 6579-6577 | 6582-6583 | 142 | SEQ ID NO: | Pre |
| | NO: 65 | | SEQ ID NO: 39 | | | | | | 100 | |
| C2 | SEQ ID | Sense | GATTTCCTGGCAGGACGCGG | 6572-6591 | TGG | 6592-6594 | 6588-6589 | 136 | SEQ ID NO: | Pre |
| | NO: 66 | | SEQ ID NO: 40 | | | | | | 101 | |
| C3 | SEQ ID | Antisense | AAGTCCTAACTTTTAAGCAC | 6563-6544 | TGG | 6543-6541 | 6546-6547 | 178 | SEQ ID NO: | Pre |
| | NO: 67 | | SEQ ID NO: 41 | | | | | | 102 | |
| C4 | SEQ ID | Sense | TCCGGAGTTCAAGACTAACC | 6650-6669 | TGG | 6670-6672 | 6666-6667 | 58 | SEQ ID NO: | Pre |
| | NO: 68 | | SEQ ID NO: 42 | | | | | | 103 | |
| C5 | SEQ ID | Antisense | AGTCTTGAACTCCGGACCTC | 6665-6646 | AGG | 6645-6643 | 6648-6649 | 76 | SEQ ID NO: | Pre |
| | NO: 69 | | SEQ ID NO: 43 | | | | | | 104 | |
| C6 | SEQ ID | Sense | CTAGGAAGGTGGATCACCTG | 6627-6646 | AGG | 6647-6649 | 6643-6644 | 81 | SEQ ID NO: | Pre |
| | NO: 70 | | SEQ ID NO: 44 | | | | | | 105 | |
| C7 | SEQ ID | Antisense | CAGGCGCGCGACACCACGCC | 6781-6762 | CGG | 6761-6759 | 6764-6765 | 22 | SEQ ID NO: | Post |
| | NO: 71 | | SEQ ID NO: 45 | | | | | | 106 | |
| C8 | SEQ ID | Sense | GAGAATCGCTTGAGCCCGGG | 6812-6831 | AGG | 6832-6834 | 6828-6829 | 86 | SEQ ID NO: | Post |
| | NO: 72 | | SEQ ID NO: 46 | | | | | | 107 | |
| C9 | SEQ ID | Antisense | CCGCAGCCTCTGGAGTAGCT | 6808-6789 | GGG | 6888-6886 | 6891-6892 | 49 | SEQ ID NO: | Post |
| | NO: 73 | | SEQ ID NO: 47 | | | | | | 108 | |
| C10 | SEQ ID | Antisense | CGGAGTGCATTGGGCGATCT | 6873-6854 | TGG | 6853-6851 | 6856-6857 | 114 | SEQ ID NO: | Post |
| | NO: 74 | | SEQ ID NO: 48 | | | | | | 109 | |
| C11 | SEQ ID | Sense | AAAGAAAAGTTAGCCGGGCG | 6746-6765 | TGG | 6766-6768 | 6762-6763 | 20 | SEQ ID NO: | Post |
| | NO: 75 | | SEQ ID NO: 49 | | | | | | 110 | |
| C12 | SEQ ID | Sense | CAAGATCGCCCAATGCACTC | 6852-6871 | CGG | 6872-6874 | 6868-6869 | 126 | SEQ ID NO: | Post |
| | NO: 76 | | SEQ ID NO: 50 | | | | | | 111 | |
| C13 | SEQ ID | Antisense | TTTCAAGCCGTGGCGTAAC | 6221-6203 | TGG | 6202-6200 | 6205-6206 | 519 | SEQ ID NO: | Pre |
| | NO: 77 | | SEQ ID NO: 51 | | | | | | 112 | |
| C14 | SEQ ID | Sense | GACGCCCATTTTGCGGACC | 6084-6102 | TGG | 6103-6105 | 6099-6100 | 625 | HO ID NO: | Pre |
| | NO: 78 | | SEQ ID NO: 52 | | | | | | 113 | |
| C15 | SEQ ID | Sense | AGTTACGCCACGGCTTGAA | 6202-6220 | AGG | 6221-6223 | 6217-6218 | 507 | SEQ ID NO: | Pre |
| | NO: 79 | | SEQ ID NO: 53 | | | | | | 114 | |
| C16 | SEQ ID | Antisense | ATACCATGTCCTCCCCTTG | 6283-6265 | AGG | 6264-6262 | 6267-6268 | 457 | SEQ ID NOs: | Pre |
| | NO: 80 | | SEQ ID NO: 54 | | | | | | 115 and 116 | |
| C17 | SEQ ID | Antisense | ATAATCCCAGCTACTCGGG | 7251-7233 | AGG | 7232-7230 | 7235-7236 | 493 | SEQ ID NO: | Post |
| | NO: 81 | | SEQ ID NO: 55 | | | | | | 117 | |
| C18 | SEQ ID | Sense | GTCTCGAACTCCCAACCTC | 7305-7323 | AGG | 7324-7326 | 7320-7321 | 578 | SEQ ID NO: | Post |
| | NO: 82 | | SEQ ID NO: 56 | | | | | | 118 | |
| C19 | SEQ ID | Antisense | CACTTTGGGAGGGCGAGGT | 7355-7337 | GGG | 7336-7334 | 7339-7340 | 597 | SEQ ID NO: | Post |
| | NO: 83 | | SEQ ID NO: 57 | | | | | | 119 | |
| C20 | SEQ ID | Antisense | TCCAGCCTGGGCAACAAGA | 7161-7143 | GGG | 7142-7140 | 7145-7146 | 403 | SEQ ID NO: | Post |
| | NO: 84 | | SEQ ID NO: 58 | | | | | | 1120 | |

### EXAMPLE 3

### Deletion of the FXN Intronic GAA Repeats in YG8R Fibroblasts

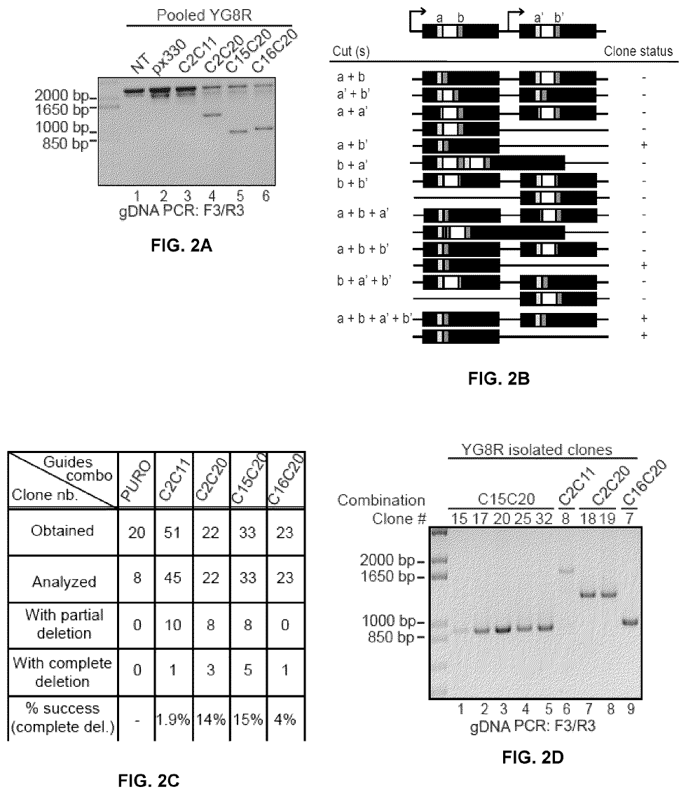

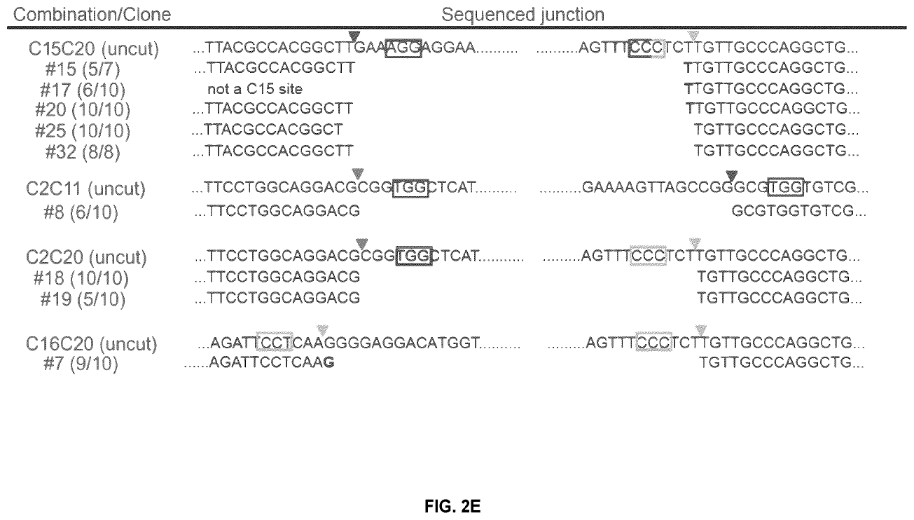

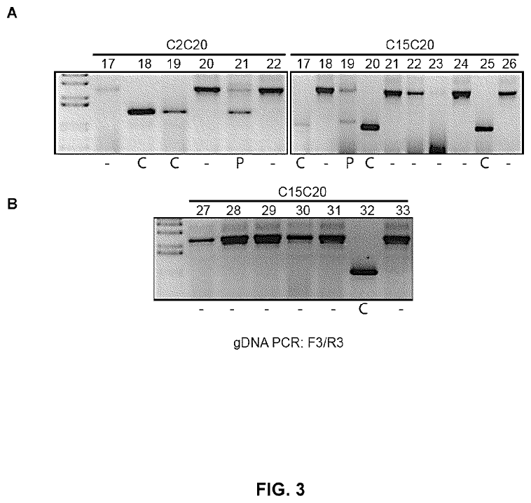

Some gRNA pairs were selected and were cloned into pxPuro, which shares similarities with px330 but contains a puromycin gene for selection. These new plasmids were retested in YG8R cells (FIG. 2A). Following detection of the corrected PCR amplicon in the puromycin resistant cell population, cells were amplified as individual clones. Since the human FRDA FXN transgene is in tandem copies in YG8R cells, there are several possible rearrangements following deletions with a pair of gRNAs, as shown in FIG. 2B. Positive clones are described as clones with a complete deletion of the GAA repeats in both tandem copies, i.e., the amplicons obtained with primers F3 and R3 did not contain the 2070 and the 2394 bp bands. Pair of gRNAs C2C20 and 015C20 gave the highest percentages of success (14% and 15% respectively) of complete deletions (FIG. 2C). Partial deletion status was attributed when one of the GAA band was still present in the amplicon (FIG. 3). Taking into account the deletion of only one of the two GAA repeats, the percentages of clones with a deletion could have been much higher: 21.6% (11/51) for C2C11, 50% (11/22) for C2C20 and 39.4% (13/33) for C15C20 (FIG. 2C). As shown in FIG. 2D, amplification of clones with a deletion using the F3/R3 primer set revealed only one band, missing the deleted section and having a size that depended on the specific gRNA pair used. The sequencing of the amplified F3/R3 amplicons for nine (9) YG8R clones (FIG. 2E) showed mostly cuts at the expected sites for SpCas9, which is 3-nucleotides upstream of its PAM. Sequence alignment (FIG. 3) showed significant identity close to the cut sites (pre- and post-GAA) and confirmed that the method, in combination with the NHEJ, is precise and reliable.

### EXAMPLE 4

### Protein Analysis IN YG8R Clones

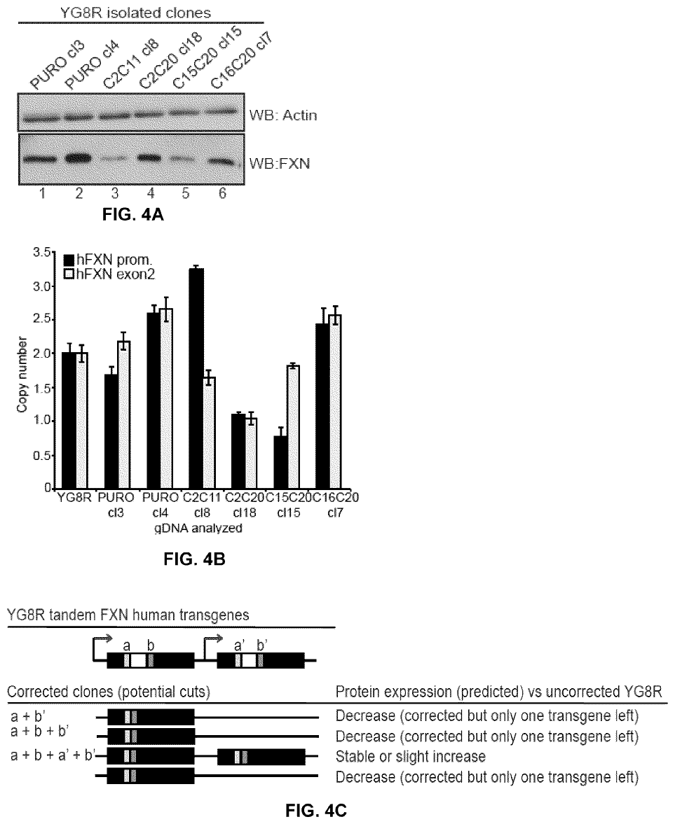

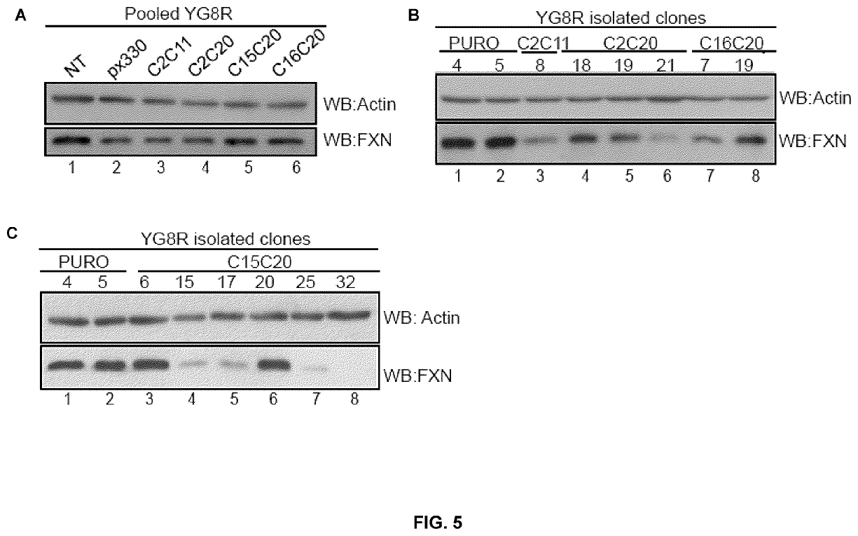

FXN protein levels were thus analyzed in samples from a heterogeneous gRNA/SpCas9 transfected YG8R cell population (FIG. 5A) and puromycin selected YG8R clones with GAA repeats from both transgenes deleted (FIG. 4A and FIGS. 5B, C). No significant differences were found following analysis of FXN protein levels extracted from the heterogeneous YG8R population (FIG. 5A, lanes 3-6). However, significant differences in FXN protein levels were observed between control clones, identified as PURO-4 and PUR-5 (FIG. 4A and FIGS. 5B, C; lanes 1 and 2), and corrected clones (FIG. 4A; lanes 3-6 and FIGS. 5B, C, lanes 3-8). Surprisingly, the FXN protein expression in most of the clones was decreased compared to the controls, which are YG8R cells transfected with a plasmid encoding the SpCas9_P2A_puromycin but missing gRNAs, and expanded as clones as well. A few clones showed no significant differences, as their FXN protein expression stays constant despite their positive clone status (i.e., deletion of GAA repeats in both transgenes). We hypothesized that for most of the positive clones, a deletion from the “a” site to the “ b′” site (FIG. 2B and FIG. 4C) removed the constitutive promoter of the second transgene, therefore reducing significantly the overall expression of the human FXN in those cells. A copy number analysis of the YG8R clones revealed that despite no evidence of residual GAA repeat (FIG. 2D), some clones did not show any changes in their FXN copy number. Other clones appeared to have lost part of the transgene while keeping another part (FIG. 4B, C15C20-15). A significant decrease in the copy number for both the promoter and the exon 2 region was only observed for the C2C20-18 clone (FIG. 4B). A stable or a slight increase of the FXN protein expression in YG8R clones could be attributed to a “a+b+a′+b′” case (FIG. 4C), which is a rare event.

The surprising initial in vitro results in YG8R fibroblasts (where a reduction, rather than an increase in FXN expression level was generally detected) is explained by the presence of two FRDA transgenes in tandem (one with about 82 GAA repeats and the other with about 190 GAA repeats) in the YG8R mouse genome. Some gRNA pairs tested frequently removed not only the GAA repeats but also (through NHEJ) one complete copy of the hFXN transgene. Since only one functioning complete FXN transgene (including the promoter region) remained, no significant change in FXN levels or reduced FXN expression (compared with the untreated YG8R cells expressing FXN from two copies of the hFXN transgene) was detected.

### EXAMPLE 5

### Deletion of the FXN Intronic GAA Repeats in YG8SR Fibroblasts

Recently a new mouse model derived from the YG8R model has been described. During the course of breeding, some YG8R mice have lost one of the human transgene (27). This new model called YG8sR presents more severe symptoms than the original mouse model, including significant behavioral deficits, together with some level of glucose intolerance and insulin hypersensitivity. These symptoms are also associated with significantly reduced expression of FAST-1 and FXN, and the presence of pathological vacuoles within neurons of the dorsal root ganglia (DRG). The YG8sR model thus represents more closely the symptoms observed in more severely affected FRDA subjects.

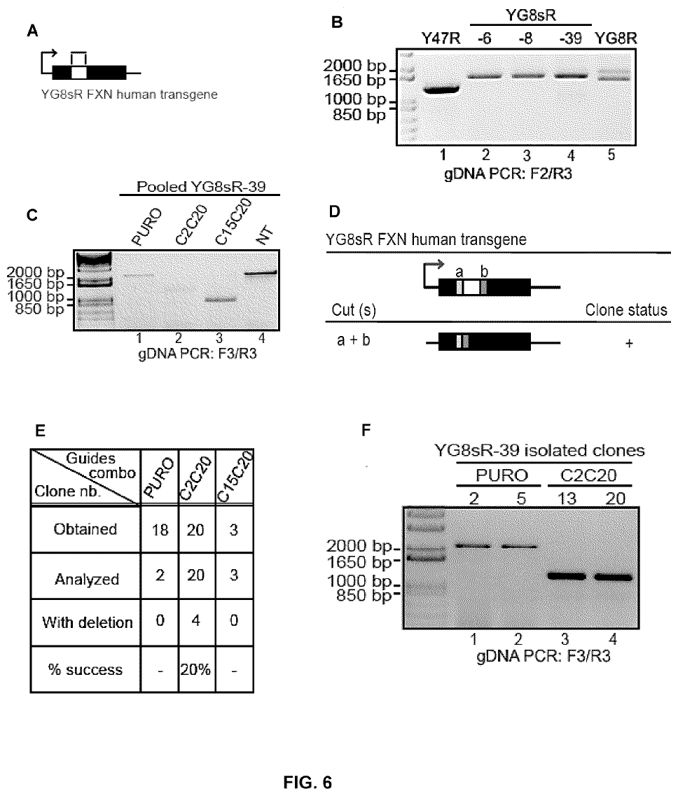

Three (3) new mouse fibroblast cell lines (called YG8sR-6, YG8sR-8 and YG8sR-39) derived from 3 different YG8sR mice were used for further experiments. Each cell line contained only one copy of the human FXN transgene with about 190 GAA repeats within intron 1 (28) (FIG. 5A). As shown in FIG. 6B, the F3/R3 primer set allowed differentiating easily the 3 YG8sR cell lines (6, 8 or 39) from the Y47R cell line (a mouse fibroblast cell line with a human FXN transgene containing a normal number of GAA repeat) and from the YG8R, which contains two copies of the human FXN transgene (i.e., two different band sizes observed by PCR amplification). YG8sR cell lines were transfected with a Cas9-encoding plasmid and two different effective pairs of gRNAs previously identified in YG8R experiments (Example 2). YG8sR-39 transfected cells were selected over YG8sR-6 and YG8sR-8 for clonal expansion (FIG. 6C) but correction with the C2C20 and the C15C20 gRNA combinations worked also in these two cells lines.

Since the YG8sR cells contain only one copy of a mutated human FXN transgene, only one rearrangement is possible by NHEJ recombination following cuts on both sides of the GAA repeat expansion (FIG. 6D). Upon expansion of the isolated YG8sR-39 clones (hereinafter generally referred to as YG8sR), 20 clones were identified, (out of 5 96-well plates seeded post-transfection and post-selection with puromycin), for the C2C20 gRNA combination, and 3 clones, for the C15C20 gRNA combination (FIG. 6E). Out of the 20 C2C20 clones, 4 clones (C2C20-13, 15, 18 and 20) were found positive, presenting a single PCR amplification product of the appropriate size following PCR amplification of genomic DNA with the F3/R3 primer set (see for example FIG. 6F showing typical results for identified positive clones) None of the C15C20 clones identified had the deletion of the GAA repeat expansion.

### EXAMPLE 6

### Identification of YG8SR Clones Expressing High Amounts of FXN Protein

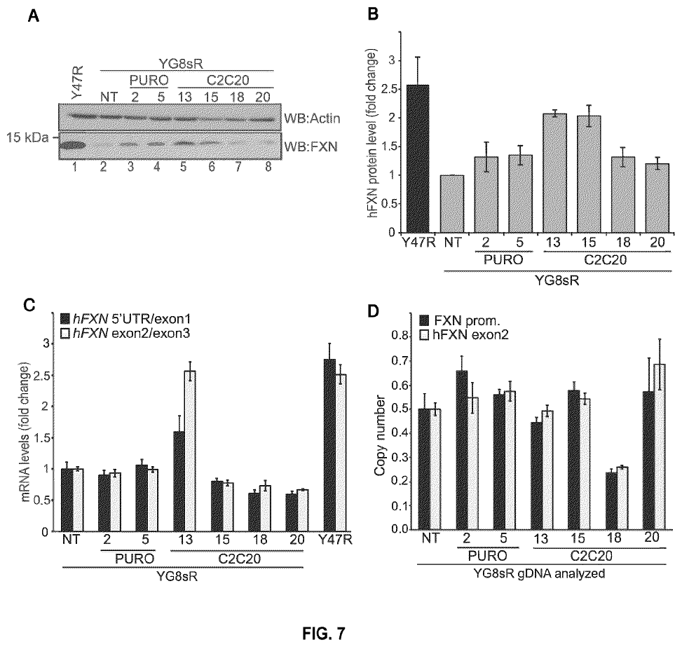

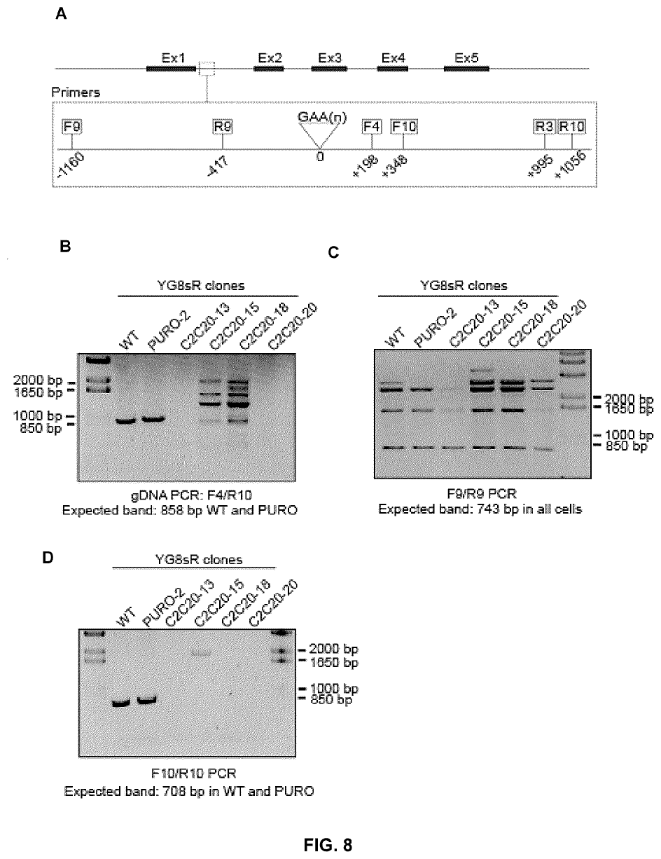

Analysis of YG8sR C2C20 clones. Protein extracts by western blot revealed an increase in FXN protein levels in two C2C20 clones (FIG. 7A, lanes 5 and 6 and FIG. 7B), however lower than in the Y47R cell line. An increase in hFXN transcriptional level was confirmed for the C2C20 clone 13, but not for clone 15 (FIG. 7C, hFXN 5′UTR/exon 1 and hFXN exon2/exon3). High FXN transcript levels were observed in Y47R cells (FIG. 7C). Genomic profile analysis of the different YG8sR C2C20 clones with different primers sets revealed discrepancies between expected and obtained PCR band profiles (FIG. 8). For example, unexpected bands appeared in the PCR made with the F4/R10 primer set for C2C20-15 and C2C20-18 clones when all samples where processed at the same time in the same conditions (FIG. 8B).

The copy number of hFXN transgene in C2C20 clones was also measured. As expected no change was found, in almost all clones, compared to the YG8sR untreated population (FIG. 7D). However, clone C2C20-18 showed a decrease by half of the copy number compared to YG8sR and other clone cell populations. Therefore, the copy number in mouse YG8sR fibroblasts is estimated to be below 1, some somatic mosaicism has indeed been initially reported (30).

### EXAMPLE 7

### Electroporation of Plasmid DNA into the Tibialis Anterior Shows in Vivo Correction

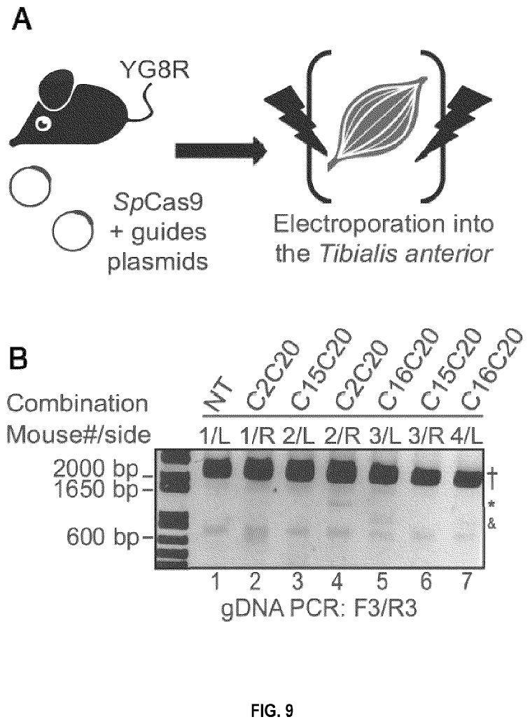

Three gRNA combinations were tested in vivo. Briefly, plasmids coding for SpCas9 and a pair of gRNAs (either C2C20, C15C20 or C16C20) were electrotransfered into the Tibialis anterior (TA) of YG8R mouse muscles (FIG. 9A). PCR was performed using the F3/R3 primer set to confirm the presence of expected PCR products (in which the GAA trinucleotide repeats have been removed, FIG. 9B, lanes 4, 5 and 7).

### EXAMPLE 8

### AAV-Encoded S. aureus cas9 Plasmid Generate cuts in Mouse Fibroblasts

As FRDA is a neuro-muscular degenerative disease involving mainly the brain, the spinal ganglia, the heart and the pancreas, a viral vector was used to deliver pairs of gRNAs in target tissues in vivo. The gRNAs were redesigned to provide an adeno-associated virus (AAV) encoding the recently available S. aureus (Sa) Cas9 protein, which requires a NNGRRT PAM sequence (31). Target sequences were thus adjusted in order to be recognized by the humanized S. aureus Cas9 (see Table 6 below).

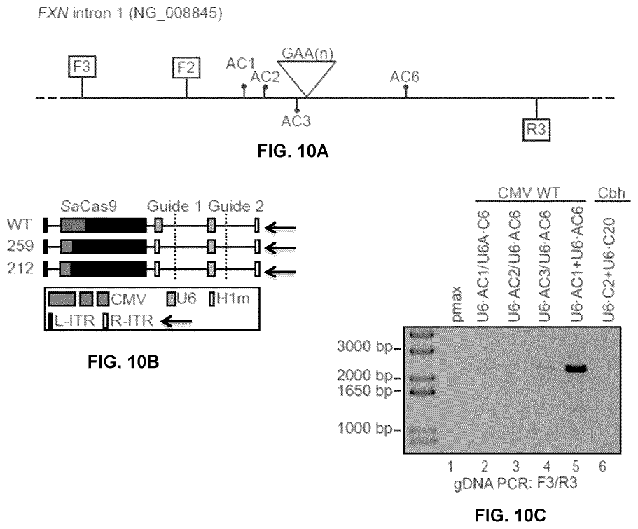

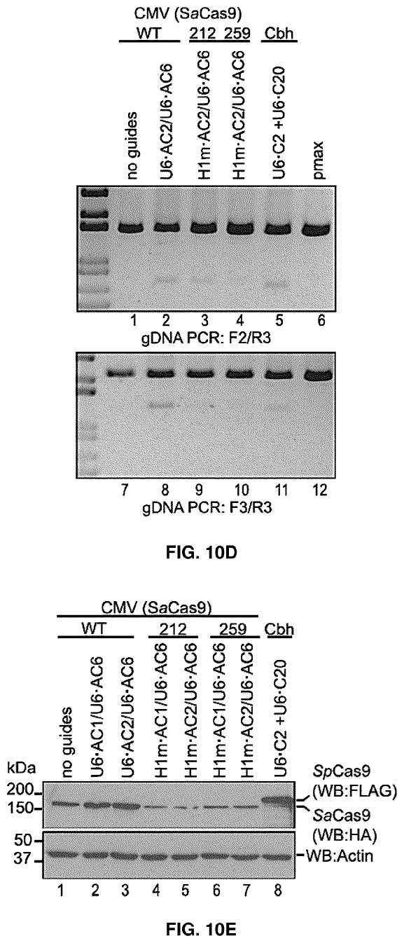

SaCas9 PAM sequences located close to previously identified SpCas9 PAMs were selected, i.e., the C2 and C20 sites (FIG. 10A). The px601 vector (22) was modified to introduce another pol III promoter (U6 or Him) and two SaCas9 tracrRNA sequences, in order to express 2 SaCas9 gRNAs from the same AAV. To do so, the size of the CMV promoter (32) was reduced (FIG. 10B). Combinations of gRNAs, transcribed from the U6 pol III promoter and SaCas9, transcribed from the non-truncated CMV promoter, targeting the AC1, AC2 or AC3 and the AC6 sites were shown to successfully cut intron 1 of the human FXN gene in cultured YG8sR (FIGS. 10C and D), and YG8R fibroblasts. Indeed, following amplification with F3/R3 primers, the predicted amplicon size representing the FXN gene in which the GAA repeats have been deleted was detected (FIG. 10C, lanes 2 and 3). AC2 and AC6 gRNAs were selected for further experiments to see whether introduction of DSBs was reduced when gRNAs were expressed from a H1m promoter (H1 “minimal”, 95 bp (32)) as opposed to the U6 promoter. No significant difference was observed (FIG. 10D, lanes 3/9 or 4/10) despite the lower amount of SaCas9 produced from the truncated CMV 212 or 259 promoter (FIG. 10E, lanes 4-7).

TABLE 6
Pre- and post-GAA repeat target sequences for S. aureus Cas9.

| | | | | gRNA | | | | Distance of | | |
|---|---|---|---|---|---|---|---|---|---|---|
| | | | | target | | | | cut from | | |
| | gRNA | | | sequence | | | | first or | Sequence | Pre- |
| | sequence | | gRNA target | gene | | PAM gene | Cut site | last nuc- | removed | or |
| | (SEQ ID | | sequence (5′-3′) | position | | position | gene | leotide in | (SEQ | post- |
| ID | NO.) | Strand | SEQ ID NO. | (5′-3′) | PAM | (5′-3′) | position | GAA repeat | ID NO.) | GAA |
| AC1 | SEQ ID | Sense | TAAAAGTTAGGACTTAGAAA | 6549-6568 | ATGGAT | 6569-6574 | 6565-6566 | 159 | SEQ ID | Pre |
| | NO: 85 | | SEQ ID NO: 59 | | | | | | NO: 121 | |
| AC2 | SEQ ID | Sense | ACTTTGGGAGGCCTAGGAAG | 6615-6634 | GTGGAT | 6635-6640 | 6631-6632 | 93 | SEQ ID | Pre |
| | NO: 86 | | SEQ ID NO: 60 | | | | | | NO: 122 | |
| AC3 | SEQ ID | Antisense | TTTGTATTTTTTAGTAGATA | 6711-6692 | CTGGGT | 6691-6686 | 6694-6695 | 30 | SEQ ID | Pre |
| | NO: 87 | | SEQ ID NO: 61 | | | | | | NO: 123 | |
| AC4 | SEQ ID | Antisense | GCCGCAGCCTCTGGAGTAGC | 6809-6790 | TGGGAT | 6789-6784 | 6792-6793 | 50 | SEQ ID | Post |
| | NO: 88 | | SEQ ID NO: 62 | | | | | | NO: 124 | |
| ACS | SEQ ID | Antisense | CCCATGCTGTCCACACAGGC | 7093-7074 | AGGGGT | 7078-7073 | 7076-7077 | 334 | SEQ ID | Post |
| | NO: 89 | | SEQ ID NO: 63 | | | | | | NO: 125 | |
| AC6 | SEQ ID | Sense | TTCCCTCTTGTTGCCCAGGC | 7138-7157 | TGGAGT | 7158-7163 | 7154-7155 | 412 | SEQ ID | Post |
| | NO: 90 | | SEQ ID NO: 64 | | | | | | NO: 126 | |

### EXAMPLE 9

### Single Intravenous Injection of AAV Vectors Coding for SPCAS9 and gRNAS in 1 Month-Old YG8SR Mice Enables Removal of GAA Repeats Intron 1 and Correction of FXN Gene in Liver Cells

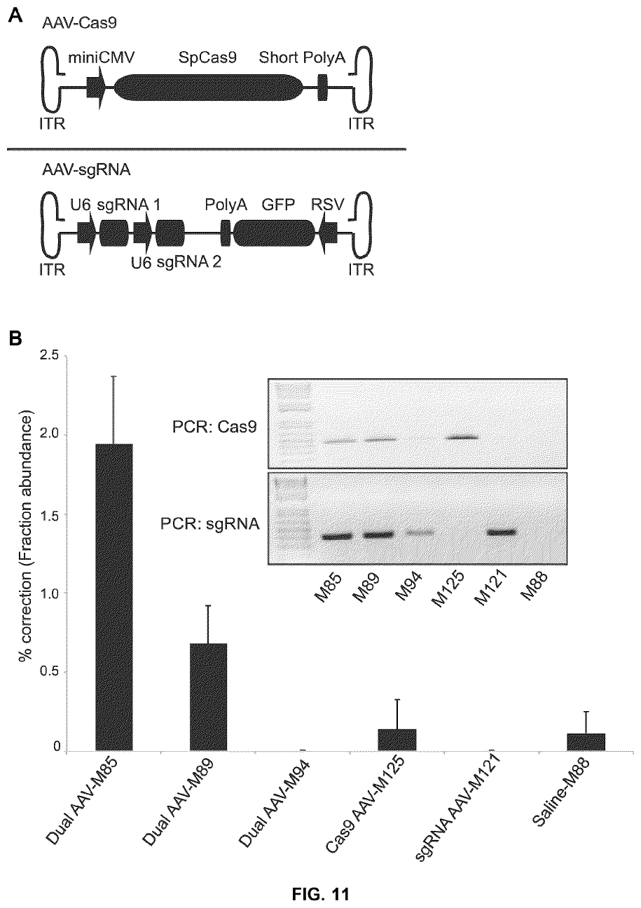

In vivo correction of intron 1 of the FXN gene using the CRISPR/Cas system was further assessed the YG8sR mouse model. Two AAV viruses were used (FIG. 11A); one coding for the SpCas9 (32) and the other for the gRNA combination C2C20 ((32) for the backbone). Both viruses were PHP.B serotyped (52).

AAV viruses were injected in one-month old mice. The mice were euthanized one month later. Genomic DNA was extracted from brain, medulla, spinal cord, dorsal root ganglia, liver, heart, Tibialis anterior and pancreas. DNA carrying the SpCas9 gene and the RSV promoter (from the gRNA plasmid) were detected in all analyzed tissues, including the brain. A digital droplet PCR approach was used to detect the correction.

Analysis reveals about 0.6-2% correction in the liver and lower percentages in other tissues. It is expected that longer infection periods will increase the number of corrected cells (FIG. 11B). Such experiments using longer infection periods are presently ongoing.

### EXAMPLE 10

### Correction of GAA Trinucleotide Repeats in Intron 1 of the FXN Gene in Human FRDA Primary Fibroblasts

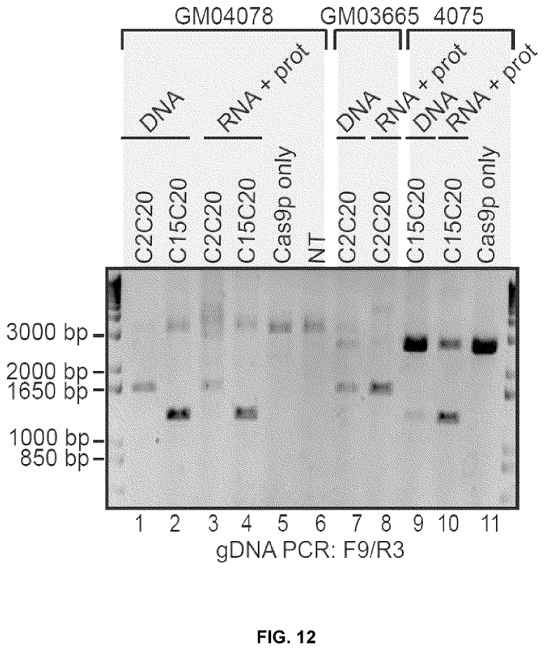

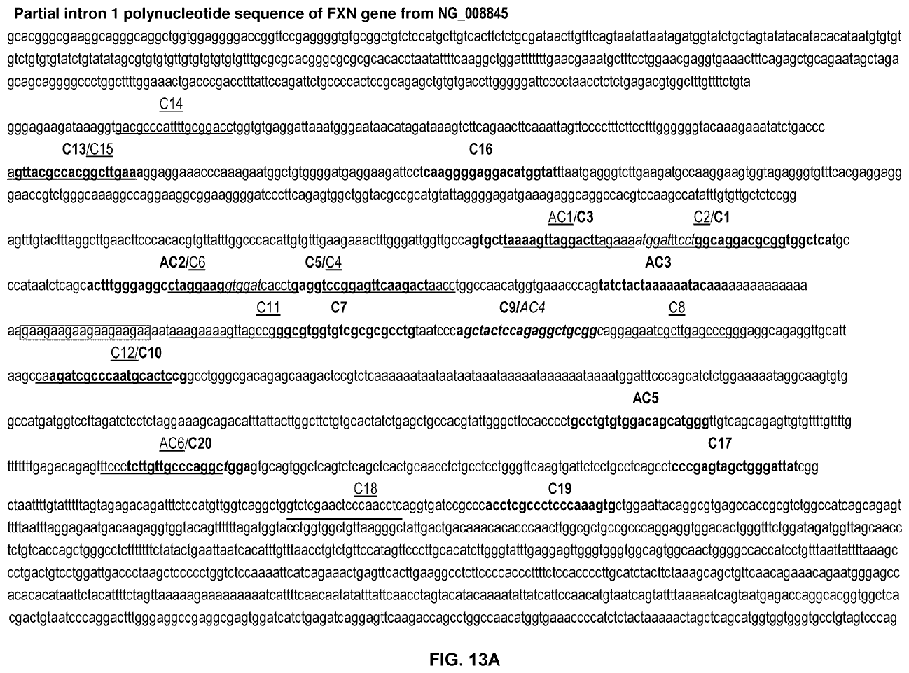

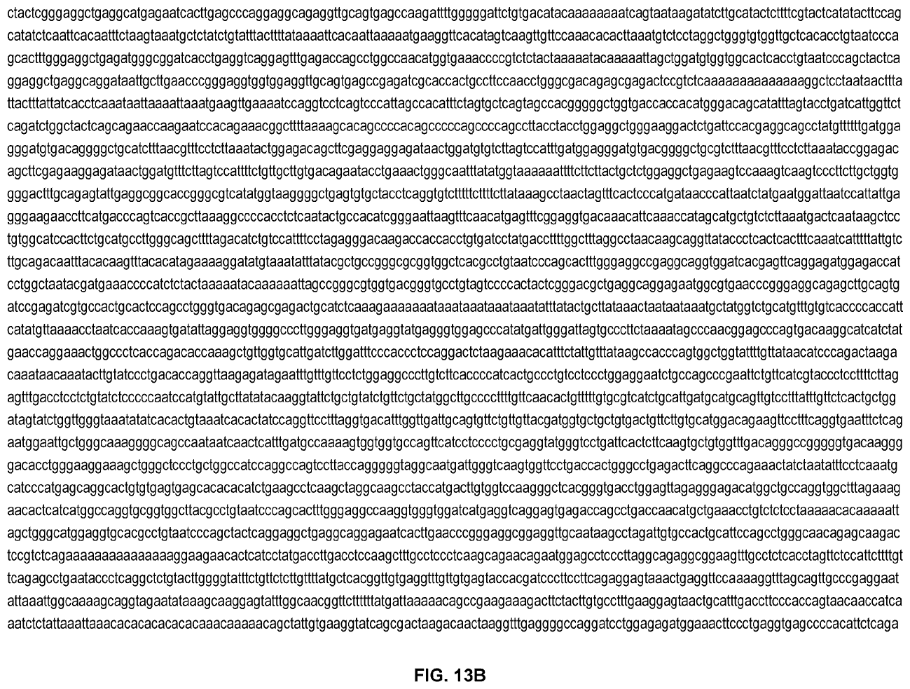

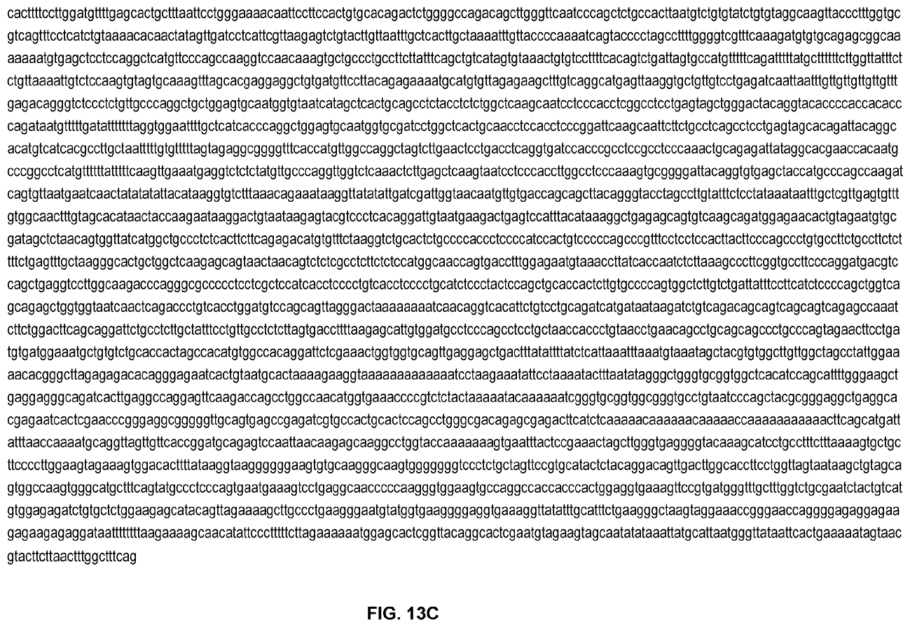

The efficiency of the method was further tested in human primary fibroblasts of FRDA patients. Two different techniques were used to achieve correction of the FXN gene using the CRISPR/Cas system: 1) nucleofection of SpCas9 and gRNA expression plasmids; or 2) nucleofection of a ribonucleoproteic complex (SpCas9 protein and gRNAs). Both methods allowed to remove GM repeats and correct the FXN gene, resulting in smaller amplicons (See FIG. 12).

The scope of the claims should not be limited by the preferred embodiments set forth in the examples, but should be given the broadest interpretation consistent with the description as a whole.

### EXAMPLE 11

### C. jejuni can be used to Delete GAA Repeats frome Intron 1 of the FXN Gene and its Small Size Gene Allows Packaging of Optimized Molecular Components in a Single AAV Vector

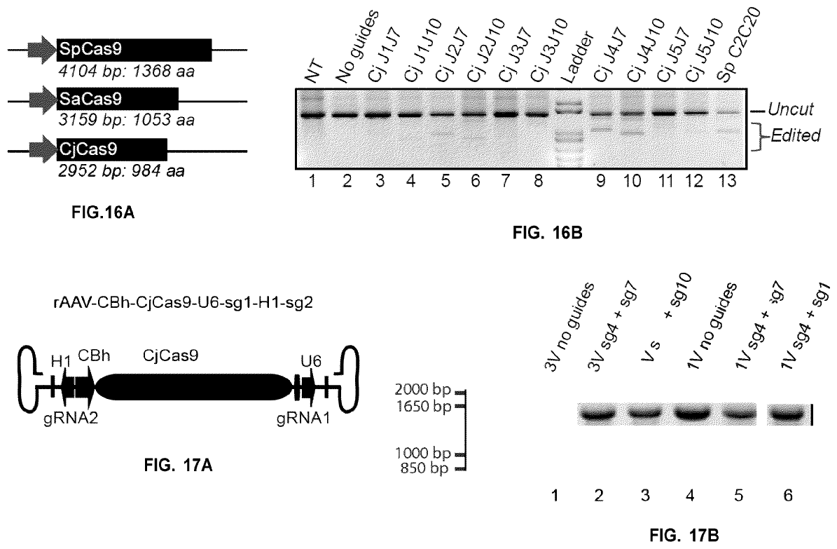

The CjCas9 (SEQ ID NO: 155, 156 or 157) was selected because of its smaller gene size compared to SpCas9 and SaCas9 (FIG. 16A). CjCas9 PAM sequences located close to previously identified SpCas9 PAMs were selected, i.e., the C2 and C20 sites (FIG. 10A). Preliminary tests using separated plasmids were performed in 293T cells using all possible combinations from 5 pre-GAA and 5 post-GAA targets (data not shown). Most efficient combinations were retested in 293T (FIG. 16B) and in YG8sR cells (data not shown). Those subsequent investigations allowed us to select Cj4Cj7 and Cj4Cj10 combinations as best CjCas9 gRNAs for the deletion of the GAA repeats. These combinations were compared to our standard, the SpCas9 C2C20 combination as similar PCR amplification were seen for the edited molecules (FIG. 16B).

A new AAV vector was constructed to introduce the CjCas9 gene (amplified from the pRGEN-CMV-CjCas9 plasmid (Addgene #89752)) under the control of a CBh promoter. A WPRE sequence was added to enhance expression. Two gRNAs can be expressed at the same time; one from a human U6 promoter and the other from a minimal H1 promoter (FIG. 17A). These constructs were tested in vitro in 293T cells and expected bands were detected corresponding to the deletion of the GAA repeat (FIG. 17B). Further investigations in YG8sR cells, as well as the production of the AAVs particles for in vivo studies are ongoing.

### EXAMPLE 12

### Preferred Target Regions for Deletion of GAA Repeats were Identified within Pre- and Post-GAA Regions

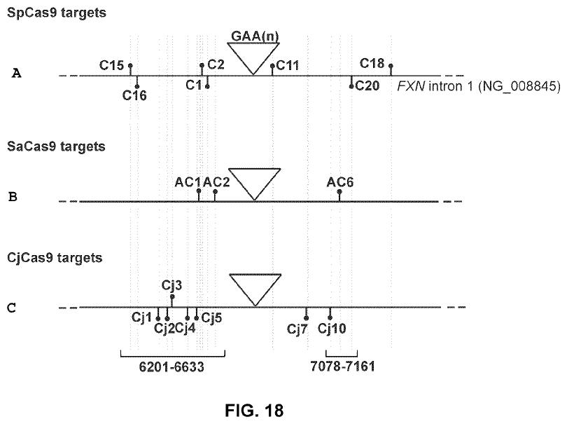

The best pre-GAA region (see FIG. 18) was identified between nucleotides 6201 and 6633 of the FXN gene (NG_008845). A subregion of particular interest was further identified between nucleotides 6594 and 6633 of the FXN gene (NG_008845). The best post post-GAA region (FIG.18) was identified between nucleotides 7078 and 7161 of the FXN gene (NG_008845). A further subregion of particular interest was determined between nucleotides 6973 and 7163 of the FXN gene. These regions contain the most efficient gRNAs identified in our investigations for SpCas9, SaCas9 and CjCas9 in both 293T and YG8sR cells. These regions may be more suitable/accessible for CRISPR nucleases such as Cas9 nucleases or more likely prone to repair by NHEJ.

Table 8 below summarizes the gRNAs tested and their efficiency, together with a CRISPR nuclease, in targeting and cutting the FXN gene.

TABLE 7
Pre- and post-GAA repeat target sequences for C. jejuni Cas9.

| | | | | | | | | Distance | | |
|---|---|---|---|---|---|---|---|---|---|---|
| | | | | gRNA | | | | of cut | | |
| | | | | target | | | | from first | | |
| | gRNA | | | sequence | | | | or last | Sequence | Pre- |
| | sequence | | gRNA target | gene | | PAM gene | Cut site | nucleotide | removed | or |
| | (SEQ. | | sequence (5′-3′) | position | | position | gene | in GAA | (SEQ | post- |
| ID | ID NO.) | Strand | SEQ ID NO. | (5′-3′) | PAM | (5′-3′) | position | repeat | ID NO.) | GAA |
| Cj1 | SEQ ID | Anti- | CTTTCATCTCCCCTAATACATG | 6422-6401 | CGGCGTAC | 6400-6393 | 6403-6404 | 321 | 158 | Pre |
| | NO: 197 | sense | SEQ ID NO: 144 | | | | | | | |
| Cj2 | SEQ ID | Anti- | GTGGCCTGCCTCTTTCATCTCC | 6433-6412 | CCTAATAC | 6411-6404 | 6414-6415 | 310 | 159 | Pre |
| | NO: 198 | sense | SEQ ID NO: 145 | | | | | | | |
| Cj3 | SEQ ID | Sense | CATATTTGTGTTGCTCTCCGGA | 6442-6463 | GTTTGTAC | 6464-6471 | 6460-6461 | 264 | 160 | Pre |
| | NO: 199 | | SEQ ID NO: 146 | | | | | | | |
| Cj4 | SEQ ID | Anti- | TCTTCAAACACAATGTGGGCCA | 6525-6502 | AATAACAC | 6501-6494 | 6504-6505 | 220 | 161 | Pre |
| | NO: 200 | sense | SEQ ID NO: 147 | | | | | | | |
| Cj4 | SEQ ID | Anti- | GGCAACCAATCCCAAAGTTTCT | 6542-6521 | TCAAACAC | 6520-6513 | 6523-6524 | 201 | 162 | Pre |
| | NO: 201 | sense | SEQ ID NO: 148 | | | | | | | |
| Cj6 | SEQ ID | Anti- | TCCACACAGGCAGGGGTGGAAG | 7084-7063 | CCCAATAC | 7062-7055 | 7065-7066 | 323 | 163 | Post |
| | NO: 202 | sense | SEQ ID NO: 149 | | | | | | | |
| Cj7 | SEQ ID | Anti- | GAGGAGATCTAAGGACCATCAT | 7002-6981 | GGCCACAC | 6980-6973 | 6984-6985 | 241 | 164 | Post |
| | NO: 203 | sense | SEQ ID NO: 150 | | | | | | | |
| Cj8 | SEQ ID | Sense | GCAGACATTTATTACTTGGCTT | 7010-7031 | CTGTGCAC | 7032-7039 | 7029-7030 | 286 | 165 | Post |
| | NO: 204 | | SEQ ID NO: 151 | | | | | | | |
| Cj9 | SEQ ID | Anti- | GCCCAATACGTGGCAGCTCAGA | 7063-7042 | TAGTGCAC | 7041-7034 | 7044-7044 | 302 | 166 | Post |
| | NO: 205 | sense | SEQ ID NO: 152 | | | | | | | |
| Cj10 | SEQ ID | Anti- | AACTCTGCTGACAACCCATGCT | 7107-7086 | GTCCACAC | 7085-7078 | 7088-7089 | 346 | 167 | Post |
| | NO: 206 | sense | SEQ ID NO: 153 | | | | | | | |

TABLE 8
Summary of gRNAs tested and their efficiency

| | Cuts (yes (y) | |
|---|---|---|
| gRNA | or no (n)) | Efficiency |
| AC1 | y | +++ |
| AC2 | y | ++++ |
| AC3 | y | ++ |
| AC4 | n | − |
| AC5 | y | ++ |
| AC6 | y | ++++ |
| C1 | y | ++ |
| C2 | y | ++++ |
| C10 | n | − |
| C11 | y | ++ |
| C12 | y | + |
| C13 | n | − |
| C14 | n | − |
| C15 | y | +++ |
| C16 | y | +++ |
| C17 | y | ++ |
| C18 | y | +++ |
| C19 | y | +++ |
| C20 | y | ++++ |
| Cj1 | y | +++ |
| Cj2 | y | +++ |
| Cj3 | y | ++ |
| Cj4 | y | ++++ |
| Cj5 | y | +++ |
| Cj6 | n | − |
| Cj7 | y | ++++ |
| Cj8 | n | − |
| Cj9 | n | − |
| Cj10 | y | ++++ |
| C20 | y | ++++ |

gRNAs C3-C9 were also prepared and tested. Preliminary results regarding efficacy of these gRNAs were uncertain due to technical problems encountered during the tests. Accordingly their efficacy could not be determined with certainty.

TABLE 9
Sequences described herein

| SEQ | |
|---|---|
| ID | |
| NO(s) | Description |
| 1 | FXN isoform 1 (210aa) from NP_000135.2 |
| 2 | FXN isoform 2 (196aa) from NP_852090 |
| 3 | FXN isoform 3 (171aa) from NP_001155178 |
| 4 | FXN gene sequence from NCBI reference number NG_008845.2. Intron 1 extends from nts 5644 to nts15822. |
| 5-38 | Primer sequences listed in Example 1 |
| 39-64 | gRNA target sequences in FXN intron 1 gene (Tables 4 and 5) |
| 65-90 | gRNA RNA sequences corresponding to the target sequences of SEQ ID NOs: 30-54 listed in Tables 5 and 6 |
| 91 | S. pyogenes Cas9 RNA recognition sequence/scaffold sequence (derived from TracrRNA/crRNA) |
| 92 | S. aureus Cas9 RNA recognition sequence/scaffold sequence (derived from tracrRNA) |
| 93 | recognition sequence/scaffold sequence from Cpf1 tracrRNA |
| 94 | Protein sequence of humanized Cas9 from S. pyogenes (without NLS and without TAG) |
| 95 | Protein sequence of humanized Cas9 from S. pyogenes (with NLS and without TAG) |
| 96 | Protein sequence of humanized Cas9 from S. pyogenes (with NLS and with TAG, from Addgene plasmid |
| | #71814) |
| 97 | Protein sequence of humanized Cas9 from S. aureus (without NLS and without TAG) |
| 98 | Protein sequence of humanized Cas9 from S. aureus (with NLS and without TAG) |
| 99 | Protein sequence of humanized Cas9 from S. aureus (with NLS and with TAG, from Addgene plasmid |
| | #61591) |
| 100-126 | Polynucleotide sequence removed by Cas9/gRNAs in intron 1 of the FXN gene. |
| | SEQ ID NOs: 100-114 (gRNAs C1 to C15); SEQ ID NOs: 115 and 116 (gRNA C16-alternative cuts detected); |
| | SEQ ID NO: 117-120 (gRNAs C17-C20); and SEQ ID NOs: 121-126 (gRNAs AC1-AC6). |
| 127-130 | Promoter polynucleotide sequences for expressing gRNAs and CRISPR nucleases (see Example 8) |
| 131-137 | Partial sequences of corrected intron 1 of FXN gene following cuts with gRNA combinations C15C20 (SEQ ID |
| | NOs: 131-133); C2C11 (SEQ ID NO: 134); C2C20 (SEQ ID NOs: 135 and 136) and C16C20 (SEQ ID NO: |
| | 137). See also FIGS. 14A-D. |
| 138-142 | Partial sequences of corrected intron 1 of FXN gene following cuts with gRNA combinations C15C18 (SEQ ID |
| | NO: 138); C16C18 (SEQ ID NO: 139); C1C20 (SEQ ID NO: 140); AC1AC6 (SEQ ID NO: 141); and AC2AC6 |
| | (SEQ ID NO: 142). See also FIGS. 15A-E. |
| 143 | Forward primer F1 used to amplify upstream of the pre-GAA repeat (see Table 4) |
| 144-153 | gRNA target sequence/gRNA DNA sequence for gRNAs Cj1-Cj10 (see Table 7) |
| 154 | Cas9 recognition sequence from C. jejuni (i.e., gRNA scaffold sequence derived from crRNA and tracrRNA) |
| 155 | Humanized Cas9 protein sequence from C. jejuni (without NLS and without TAG) |
| 156 | Humanized Cas9 protein sequence from C. jejuni (NLS and without TAG) |
| 157 | Protein sequence of humanized high specific Cas9 from C. jejuni (with NLS and with TAG; from Addgene |
| | plasmid #89752) (1003 aa)-HA TAG (C-term) |
| 158-167 | Nucleotide sequence removed following cut by each of Cj1-Cj10 in intron 1 of FXN gene |
| 168 | H1 minimal promoter sequence |
| 169 | CBh (or CBA hybrid intron): CBA promoter with a hybrid intron composed of a 5′ donor splice site from the |
| | chicken β-actin 5′ UTR and a 3′ acceptor splice site from MVM (Minute virus of mice). |
| 170 | WPREL sequence: Sequence containing SV40 late poly A (135 bp) and Woodchuck post transcriptional region |
| | gamma and alpha elements (247 bp) |
| 171-195 | Partial sequence of frataxin intron 1 following cuts with Cj1Cj6 (SEQ ID NO: 171); Cj1Cj7 (SEQ ID NO: 172); |
| | Cj1Cj8 (SEQ ID NO: 173); Cj1Cj9 (SEQ ID NO: 174); Cj1Cj10 (SEQ ID NO: 175); Cj2Cj6 (SEQ ID NO: 176); |
| | Cj2Cj7 (SEQ ID NO: 177); Cj2Cj8 (SEQ ID NO: 178); Cj2Cj9 (SEQ ID NO: 179); Cj2Cj10 (SEQ ID NO: 180); |
| | Cj3Cj6 (SEQ ID NO: 181); Cj3Cj7 (SEQ ID NO: 182); Cj3Cj8 (SEQ ID NO: 183); Cj3Cj9 (SEQ ID NO: 184); |
| | Cj3Cj10 (SEQ ID NO: 185); Cj4Cj6 (SEQ ID NO: 186); Cj4Cj7 (SEQ ID NO: 187); Cj4Cj8 (SEQ ID NO: 188); |
| | Cj4Cj9 (SEQ ID NO: 189); Cj4Cj10 (SEQ ID NO: 190); Cj5Cj6 (SEQ ID NO: 191); Cj5Cj7 (SEQ ID NO: 192); |
| | Cj5Cj8 (SEQ ID NO: 193); Cj5Cj9 (SEQ ID NO: 194); and Cj5Cj10 (SEQ ID NO: 195) |
| 196 | WPRE sequence comprising alpha, beta and gamma elements |
| 197-206 | gRNA sequence of Cj1-Cj10 of Table 7 (CjCas9) |
| 207-208 | Fragments of human FXN gene intron 1 corresponding to effective subregions discussed in Example 12 (SEQ |
| | ID NO. 207 corresponds to a subregion upstream of GAA repeats and SEQ ID NO: 208 corresponds to a |
| | subregion downstream of GAA repeats). |
| 209-210 | Fragments of human FXN gene intron 1 corresponding to effective regions identified in FIG. 18 (pre GAA, |
| | (6201-6633, SEQ ID NO: 208) and post GAA (7078-7161, SEQ ID NO: 210) |
| 211 | Primer H1F Example 1 |

### REFERENCES

1. Babady N E, Carelle N, Wells R D, Rouault T A, Hirano M, Lynch D R, et al. Advancements in the pathophysiology of Friedreich's Ataxia and new prospects for treatments. Mol Genet Metab. 2007; 92(1-2):23-35.
2. Cooper J M, Schapira A H. Friedreich's Ataxia: disease mechanisms, antioxidant and Coenzyme Q10 therapy. Biofactors. 2003; 18(1-4):163-71.
3. Harding A E. Friedreich's ataxia: a clinical and genetic study of 90 families with an analysis of early diagnostic criteria and intrafamilial clustering of clinical features. Brain. 1981; 104(3):589-620.
4. Lynch D R, Farmer J M, Balcer L J, Wilson R B. Friedreich ataxia: effects of genetic understanding on clinical evaluation and therapy. Arch Neurol. 2002; 59(5):743-7.
5. Pandolfo M. Molecular pathogenesis of Friedreich ataxia. Arch Neurol. 1999; 56(10):1201-8.
6. Pandolfo M. Friedreich ataxia: the clinical picture. J Neurol. 2009; 256 Suppl 1:3-8.
7. Pandolfo M. Friedreich ataxia. Handbook of clinical neurology (Chapter 17)/edited by PJ Vinken and GW Bruyn. 2012; 103:275-94.
8. Campuzano V, Montermini L, Molto M D, Pianese L, Cossee M, Cavalcanti F, et aL Friedreich's ataxia: autosomal recessive disease caused by an intronic GAA triplet repeat expansion. Science. 1996; 271(5254):1423-7.
9. Pandolfo M. The molecular basis of Friedreich ataxia. Adv Exp Med Biol. 2002; 516:99-118.
10. Campuzano V, Montermini L, Lutz Y, Cova L, Hindelang C, Jiralerspong S, et al. FXN is reduced in Friedreich ataxia patients and is associated with mitochondrial membranes. Hum Mol Genet. 1997; 6(11):1771-80.
11. Pandolfo M. Iron and Friedreich ataxia. J Neural Transm Suppl. 2006(70):143-6.
12. Coppola G, Choi S H, Santos M M, Miranda C J, Tentler D, Wexler E M, et al. Gene expression profiling in FXN deficient mice: microarray evidence for significant expression changes without detectable neurodegeneration. Neurobiol Dis. 2006; 22(2):302-11.
13. Coppola G, Marmolino D, Lu D, Wang Q, Cnop M, Rai M, et al. Functional genomic analysis of FXN deficiency reveals tissue-specific alterations and identifies the PPARgamma pathway as a therapeutic target in Friedreich's ataxia. Hum Mol Genet. 2009; 18(13):2452-61.
14. Gerber J, Muhlenhoff U, Lill R. An interaction between FXN and Isu1/Nfs1 that is crucial for Fe/S cluster synthesis on Isu1. EMBO Rep. 2003; 4(9):906-11.
15. Wiedenheft B, Sternberg S H, Doudna J A. RNA-guided genetic silencing systems in bacteria and archaea. Nature. 2012; 482(7385):331-8.
16. Bhaya D, Davison M, Barrangou R. CRISPR-Cas systems in bacteria and archaea: versatile small RNAs for adaptive defense and regulation. Annu Rev Genet. 2011; 45:273-97.
17. Terns M P, Terns R M. CRISPR-based adaptive immune systems. Curr Opin Microbiol. 2011; 14(3):321-7.
18. Mali P, Yang L, Esvelt K M, Aach J, Guell M, DiCarlo J E, et al. RNA-guided human genome engineering via Cas9. Science. 2013; 339(6121):823-6.
19. Mojica FJ, Diez-Villasenor C, Garcia-Martinez J, Almendros C. Short motif sequences determine the targets of the prokaryotic CRISPR defence system. Microbiology. 2009; 155(Pt 3):733-40.
20. He Z, Proudfoot C, Mileham A, J., McLaren D G, Whitelaw B A, Lillico S G. Highly efficient targeted chromosome deletions using CRISPR/Cas9. Biotechnology and Bioengineering. 2014; online.
21. Byrne S M, Ortiz L, Mali P, Aach J, Church G M. Multi-kilobase homozygous targeted gene replacement in human induced pluripotent stem cells. Nucleic Acids Res. 2014.
22. Slaymaker I M, Gao L, Zetsche B, Scott D A, Yan W X, Zhang F. Rationally engineered Cas9 nucleases with improved specificity. Science. 2015.
23. Kleinstiver B P, Pattanayak V, Prew M S, Tsai S Q, Nguyen NT, Zheng Z, et al. High-fidelity CRISPR-Cas9 nucleases with no detectable genome-wide off-target effects. Nature. 2016.
24. Pook M A, Al-Mandawi S, Carroll C J, Cossee M, Puccio H, Lawrence L, et al. Rescue of the Friedreich's ataxia knockout mouse by human YAC transgenesis. Neurogenetics. 2001; 3(4):185-93.
25. Al-Mandawi S, Pinto R M, Ruddle P, Carroll C, Webster Z, Pook M. GAA repeat instability in Friedreich ataxia YAC transgenic mice. Genomics. 2004; 84(2):301-10.
26. Al-Mandawi S, Pinto R M, Varshney D, Lawrence L, Lowrie M B, Hughes S, et al. GAA repeat expansion mutation mouse models of Friedreich ataxia exhibit oxidative stress leading to progressive neuronal and cardiac pathology. Genomics. 2006; 88(5):580-90.
27. Virmouni SA, Ezzatizadeh V, Sandi C, Sandi M, Al-Mandawi S, Chutake Y, et al. A novel GAA repeat expansion-based mouse model of Friedreich ataxia. Disease Models & amp; Mechanisms. 2015; in press.
28. Anjomani Virmouni S, Ezzatizadeh V, Sandi C, Sandi M, Al-Mandawi S, Chutake Y, et al. A novel GAA-repeat-expansion-based mouse model of Friedreich's ataxia. Dis Model Mech. 2015; 8(3):225-35.
29. Anjomani Virmouni S, Sandi C, Al-Mandawi S, Pook M A. Cellular, molecular and functional characterisation of YAC transgenic mouse models of Friedreich ataxia. PLoS One. 2014; 9(9):e107416.
30. Virmouni S A. Genotype and phenotype characterisation of Friedreich ataxia mouse models and cells. Brunel University London library. 2013.
31. Ran F A, Cong L, Yan W X, Scott D A, Gootenberg J S, Kriz A J, et al. In vivo genome editing using Staphylococcus aureus Cas9. Nature. 2015; 520(7546):186-91.
32. Senis E, Fatouros C, Grosse S, Wiedtke E, Niopek D, Mueller AK, et al. CRISPR/Cas9-mediated genome engineering: an adeno-associated viral (AAV) vector toolbox. Biotechnology journal. 2014; 9(11):1402-12.
33. Long C, Amoasii L, Mireault A A, McAnally J R, Li H, Sanchez-Ortiz E, et al. Postnatal genome editing partially restores FXNtrophin expression in a mouse model of muscular FXNtrophy. Science. 2016; 351(6271):400-3.
34. Nelson C E, Hakim C H, Ousterout D G, Thakore P I, Moreb E A, Castellanos Rivera R M, et al. In vivo genome editing improves muscle function in a mouse model of Duchenne muscular FXNtrophy. Science. 2016; 351(6271):403-7.
35. Tabebordbar M, Zhu K, Cheng J K, Chew W L, Widrick J J, Yan W X, et al. In vivo gene editing in FXNtrophic mouse muscle and muscle stem cells. Science. 2016; 351(6271):407-11.
36. lyombe-Engembe J P, Ouellet D L, Rousseau J, Chapdelaine P, Tremblay J P. Efficient Restoration of the FXNtrophin Gene Reading Frame and Protein Structure in DMD Myoblasts Using the CinDel Method. Molecular Therapy Nucleic Acid Research. 2016; Online publication http://www. nature.com/mtna/journal/v5/n 1/fuII/mtna201558a.html.
37. Courtney D G, Moore J E, Atkinson S D, Maurizi E, Allen E H, Pedrioli D M, et al. CRISPR/Cas9 DNA cleavage at SNP-derived PAM enables both in vitro and in vivo KRT12 mutation-specific targeting. Gene Ther. 2016; 23(1):108-12.
38. Yin H, Song C Q, Dorkin J R, Zhu L J, Li Y, Wu Q, et al. Therapeutic genome editing by combined viral and non-viral delivery of CRISPR system components in vivo. Nat Biotechnol. 2016; 34(3):328-33.
39. Sachdeva M, Sachdeva N, Pal M, Gupta N, Khan I A, Majumdar M, et al. CRISPR/Cas9: molecular tool for gene therapy to target genome and epigenome in the treatment of lung cancer. Cancer Gene Ther. 2015; 22(11):509-17.
40. Li Y, Lu Y, Polak U, Lin K, Shen J, Farmer J, et al. Expanded GAA repeats impede transcription elongation through the FXN gene and induce transcriptional silencing that is restricted to the FXN locus. Hum Mol Genet 2015; 24(24):6932-43.
41. Chutake Y K, Costello W N, Lam C C, Parikh A C, Hughes T T, Michalopulos M G, et al. FXN Promoter Silencing in the Humanized Mouse Model of Friedreich Ataxia. PLoS One. 2015; 10(9):e0138437.
42. Sandi C, Pinto R M, Al-Mandawi S, Ezzatizadeh V, Barnes G, Jones S, et al. Prolonged treatment with pimelic o-aminobenzamide HDAC inhibitors ameliorates the disease phenotype of a Friedreich ataxia mouse model. Neurobiol Dis. 2011; 42(3):496-505.
43. Cong L, Ran F A, Cox D, Lin S, Barretto R, Habib N, et al. Multiplex genome engineering using CRISPR/Cas systems. Science. 2013; 339(6121):819-23.
44. Ran F A, Hsu P D, Wright J, Agarwala V, Scott D A, Zhang F. Genome engineering using the CRISPR-Cas9 system. Nature protocols. 2013; 8(11):2281-308.
45. Gray J T, Zolotukhin S. Design and construction of functional AAV vectors. Methods in molecular biology. 2011; 807:25-46.
46. Pichavant C, Chapdelaine P, Cerri D G, Bizario J C, Tremblay J P. Electrotransfer of the full-length dog FXNtrophin into mouse and FXNtrophic dog muscles. Hum Gene Ther. 2010; 21(11):1591-601.
47. Pfaffl M W. A new mathematical model for relative quantification in real-time RT-PCR. Nucleic Acids Res. 2001; 29(9):e45.
48. Bustin S A, Benes V, Garson J A, Hellemans J, Huggett J, Kubista M, et al. The MIQE guidelines: minimum information for publication of quantitative real-time PCR experiments. Clin Chem. 2009; 55(4):611-22.
49. Bustin S A, Beaulieu J F, Huggett J, Jaggi R, Kibenge F S, Olsvik P A, et al. MIQE precis: Practical implementation of minimum standard guidelines for fluorescence-based quantitative real-time PCR experiments. BMC Mol Biol. 2010; 11:74.
50. Luu-The V, Paquet N, Calvo E, Cumps J. Improved real-time RT-PCR method for high-throughput measurements using second derivative calculation and double correction. Biotechniques. 2005; 38(2):287-93.
51. Chapdelaine P, Coulombe Z, Chikh A, Gerard C, Tremblay J P. A Potential New Therapeutic Approach for Friedreich Ataxia: Induction of FXN Expression With TALE Proteins. Mol Ther Nucleic Acids. 2013; 2:e119.
52. Deverman B. E and al, Cre-dependant selection yields AAV variants for widespread gene transfer to the adult brain, Nature Biotechnology, February 2016.
53. Kumari D. et al. Repeat expansion affects both transcription initiation and elongation in Friedreich ataxia cells. Journal of Biol. Chemistry. 2011; 286(6); pp. 4209-4215.
54. Sandi C. et al. Epigenetics in Friedreich's ataxia: Challenges and opportunities for therapy. Genetics Research Int 2013, vol. 2013, Article IS 852080.
55. Sandi C. et al. Epigenetic-based therapies for Friedreich ataxia. Frontiers in Genetics. Jun. 3, 2014. Volume 5, Article 165.
56. Yandim C. et aL Gene regulation and epigenetics in Friedreich ataxia. Journal of Neurochemistry. 2013. 126(Suppl. 1); pp. 21-42.
57. De Biase I. et al. Epigenetic silencing in Friedreich ataxia is associated with depletion of CTFF (CCCTC-Binding factor) and antisense transcription. PLOS ONE. 2009. Vol. 4 (11), e7914.
58. Mohanraju, P. et al., PMID 27493190.
59. Shmakov, S et al., PMID: 26593719.
60. Zetsche, B. et al., PMID: 26422227.
61. Kleinstiver BP, Prew MS, Tsai SQ, Nguyen NT, Topkar VV, Zheng Z, Joung JK. Broadening the targeting range of Staphylococcus aureus CRISPR-Cas9 by modifying PAM recognition. Nat Biotechnol. 2015; 33(12):1293-8. doi: 10.1038/nbt.3404. PubMed PMID: 26524662; PMClD: PMC4689141.
62. Kleinstiver B P, Prew M S, Tsai S Q, Topkar V V, Nguyen N T, Zheng Z, Gonzales A P, Li Z, Peterson R T, Yeh J R, Aryee M J, Joung J K. Engineered CRISPR-Cas9 nucleases with altered PAM specificities. Nature. 2015; 523(7561):481-5. doi: 10.1038/nature14592. PubMed PMID: 26098369; PMCID: PMC4540238.
63. Shah S A, Erdmann S, Mojica F J and Garrett R A. Protospacer Recognition Motifs-Mixed Identities and Functional Diversity. 5, s.l.:RNA Biology, May 2013, Vol. 10, pp. 891-899.

1-60. (canceled)

61. A method of modifying within a cell, a frataxin (FXN) gene comprising a plurality of GAA trinucleotide repeats in an intron of said gene, the method comprising:
(a) introducing a first cut within the intron of the FXN gene creating a first intron end, wherein said first cut is located upstream of or within the plurality of GAA trinucleotide repeats;
(b) introducing a second cut within the intron of the FXN gene creating a second intron end, wherein said second cut is located downstream of or within the plurality of GAA trinucleotide repeats;
wherein upon ligation of said first and second intron ends, said FXN gene is modified and some or all of said GAA trinucleotide repeats are removed.

62. The method of claim 61, wherein the first and second cuts are introduced by providing a cell with (i) at least one CRISPR nuclease; and (ii) a pair of gRNAs consisting of (a) a first gRNA which binds to a polynucleotide sequence within the intron of the FXN gene located upstream of the plurality of GAA trinucleotide repeats for introducing a first cut; (b) a second gRNA which binds to a polynucleotide sequence within the intron of the FXN gene located downstream of the plurality of GAA trinucleotide repeats for introducing the second cut.

63. The method of claim 61, wherein the FXN gene comprises at least 70 GAA trinucleotide repeats within the intron.

64. The method of claim 61, wherein said first cut is located for the removal of between 30 and 506 nucleotides upstream of the GAA trinucleotide repeats.

65. The method of claim 61, wherein the second cut is located for the removal of between 20 and 478 nucleotides downstream of the GAA trinucleotide repeats.

66. The method of claim 61, wherein:
the first gRNA has a target sequence adjacent to a NGG PAM nucleotide sequence located within nts 6201-6633 and the second gRNA has a target sequence adjacent to a NGG PAM nucleotide sequence located within nts 7078-7161;
the first gRNA has a target sequence adjacent to a NNGRRT PAM nucleotide sequence located within nts 6201-6633 and the second gRNA has a target sequence adjacent to a NNGRRT PAM nucleotide sequence located within nts 7078-7161; or
the first gRNA has a target sequence adjacent to a NNNNRYAC PAM nucleotide sequence located within nts 6201-6633 and the second gRNA has a target sequence adjacent to a NNNNRYAC PAM nucleotide sequence located within nts 7078-7161;
wherein the nucleotide positions are given with respect to the FXN polynucleotide gene sequence set forth in GenBank NG_00845 (SEQ ID NO: 4).

67. The method of claim 61, wherein:
the first gRNA has a target sequence adjacent to a NGG PAM nucleotide sequence located within nts 6594-6633 and the second gRNA has a target sequence adjacent to a NGG PAM nucleotide sequence located within nts 6973-7163;
the first gRNA has a target sequence adjacent to a NNGRRT PAM nucleotide sequence located within nts 6594-6633 and the second gRNA has a target sequence adjacent to a NNGRRT PAM nucleotide sequence located within nts 6973-7163; or
the first gRNA has a target sequence adjacent to a NNNNRYAC PAM nucleotide sequence located within nts 6594-6633 and the second gRNA has a target sequence adjacent to a NNNNRYAC PAM nucleotide sequence located within nts 6973-7163;
wherein the nucleotide positions are given with respect to the FXN polynucleotide gene sequence set forth in GenBank NG_00845 (SEQ ID NO: 4).

68. A gRNA pair for deleting a plurality of endogenous GAA trinucleotide repeats within an intron of a FXN gene within a cell, wherein said pair consists of a first gRNA and a second gRNA, wherein (a) said first gRNA binds to a first polynucleotide sequence within the intron of the FXN gene located upstream of or within the plurality of GAA trinucleotide repeats for introducing a first cut; and (b) said second gRNA binds to a second polynucleotide sequence within the intron of the FXN gene located downstream of or within the plurality of GAA trinucleotide repeats for introducing a second cut downstream from the first cut.

69. The gRNA pair of claim 68, wherein said first cut removes between 30 and 506 nucleotides upstream of the GAA trinucleotide repeats.

70. The gRNA pair of claim 68, wherein the second cut removes between 20 and 478 nucleotides downstream of the GAA trinucleotide repeats.

71. The gRNA pair of claim 68, wherein:
the first gRNA has a target sequence adjacent to a NGG PAM nucleotide sequence located within nts 6201-6633 and the second gRNA has a target sequence adjacent to a NGG PAM nucleotide sequence located within nts 7078-7161;
the first gRNA has a target sequence adjacent to a NNGRRT PAM nucleotide sequence located within nts 6201-6633 and the second gRNA has a target sequence adjacent to a NNGRRT PAM nucleotide sequence located within nts 7078-7161; or
the first gRNA has a target sequence adjacent to a NNNNRYAC PAM nucleotide sequence located within nts 6201-6633 and the second gRNA has a target sequence adjacent to a NNNNRYAC PAM nucleotide sequence located within nts 7078-7161;
wherein the nucleotide positions are given with respect to the FXN polynucleotide gene sequence set forth in GenBank NG_00845 (SEQ ID NO: 4).

72. The gRNA pair of claim 68, wherein:
the first gRNA has a target sequence adjacent to a NGG PAM nucleotide sequence located within nts 6594-6633 and the second gRNA has a target sequence adjacent to a NGG PAM nucleotide sequence located within nts 6973-7163;
the first gRNA has a target sequence adjacent to a NNGRRT PAM nucleotide sequence located within nts 6594-6633 and the second gRNA has a target sequence adjacent to a NNGRRT PAM nucleotide sequence located within nts 6973-7163; or
the first gRNA has a target sequence adjacent to a NNNNRYAC PAM nucleotide sequence located within nts 6594-6633 and the second gRNA has a target sequence adjacent to a NNNNRYAC PAM nucleotide sequence located within nts 6973-7163;
wherein the nucleotide positions are given with respect to the FXN polynucleotide gene sequence set forth in GenBank NG_00845 (SEQ ID NO: 4).

73. A nucleic acid comprising one or more polynucleotide sequences encoding one or both members of the gRNA pair of claim 68.

74. The nucleic acid of claim 73, further comprising a sequence encoding a CRISPR nuclease.

75. A nucleic acid comprising a modified FXN gene comprising ligated first and second intron ends as defined in claim 61.

76. A vector comprising the nucleic acid of claim 73.

77. A combination of vectors comprising:
a gRNA vector comprising a first nucleic acid comprising a polynucleotide sequence encoding the first gRNA and a second nucleic acid comprising a polynucleotide sequence encoding the second gRNA, of the gRNA pair of claim 68; and
a CRISPR nuclease vector comprising a third nucleic acid comprising a polynucleotide sequence encoding one or more CRISPR nucleases.

78. A cell comprising the vector of claim 76.

79. A method for treating Friedreich ataxia in a subject, comprising modifying a FXN gene and increasing FXN expression within a cell of said subject according to the method of claim 61.

80. A method for treating Friedreich ataxia in a subject, comprising contacting a cell of the subject with (i)(a) the gRNA pair of claim 68 or one or more nucleic acids encoding said gRNA pair and (b) a CRISPR nuclease polypeptide or a nucleic acid encoding a CRISPR nuclease polypeptide.


SEQUENCE LISTING (223 sequences)

SEQ ID NO: 1 - protein, 210 aa, homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 196 aa, homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSRYVVDLSVMTGLGKTGCTPTTACPSMSCWPQSSLKP

SEQ ID NO: 3 - protein, 171 aa, homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSRLTWLLWLFHP

SEQ ID NO: 4 - DNA, 71616 bp, homo sapiens
gagaggcctctgcctgaagctcccttaatctgtcagatcacagatcaaaagctatcacacactgcccaagggaccctaaagggagccactctcagaaaataaatccaaacctccttttttctggccactgaaacgttcaataattagttcattatctacatcattcacatgtttaatatttattgactttggaattattgattactttgctgagtatcttatgaatttaatctatattaatattaaggtgatgtatcaaattgcattccagagtgtggatttgactctagtgccataatcagtctcctgggacaaacagctgtttctcttccctcattatagaaaaaaattgcccttggcaaatgtcaaagaacatccttttatcaatctctcttaccaatcaatccaagcaaatgcagtgggatttcttttccccagagttgaagtcacctcctgacaggaagttaagtctttaggcactgaatcatagcactgagctgaagcccaggactaagcaagaatgagtgagaatttggagacttaaggtttggtcatctgtagaggattgggttttgtcttgttttgtttttgttttggtgtcctgcaaggtttctcactgcccttcatcaggtaatatgccctgtccctgaactggccaattgtgttaccccattcccttagcaacagtgattgctactgaagaaacaggaggccaacaaagaatatcactcattcaccagtagagtgtctatgtcagagatattagataaacaggaaaactaacaattacattgcaggctgtggaaacagagagaggtacctaaggcacacctgtgggaatatgaaagtttggtttcataaaaggattctggaggaagtcacctctgacatttgagttgagtcctacaggatatgaagaaagtagccccatgttccggttactatggctgtgtaataaattacctaaaacatactggattaaagctatgaaaacatcttttctgcccagagattctgtgggcctgaaattcacatggaacacataaggcataccttgtttctgctccatgatgtctaaggattcagctggaagactgggaggctgggggctggaatttatctgaagaatgactcatttacatgtctactggttgaccctggctgttggctgagggggcctcagctctctctacattgtttctctactttggctagtttggatttcctcaaaacatgatggctggtcctcagtgtcagcatcccaaaaagagaatgccggacacagtggctcacacctgtaacctcagcaatttgggaggctgaggtgggaggttgcttgagttcaggagttcaagaccagcctgggcaacatagtgagactctgtctctaccaaaaatttttaaaattttaaaaatcagccaggtgtggttatgcacacctatagtcccagttacttaggaggctgaggcaggaggatcacttgattccaggaggtcaaggctgctgtgagctatgaccacaccactgcatttcagcctgggtaacagaatgagactctgtttcaaacaaacaaaaacccaaaaaaaaaaaaaagagagagagagggagttagaaggaagatgcatcatttttatgacctggacttggaagtcaccaagcagcacttctgcagtaccctgttggttggaatagttgtagcccaaacccgaattcgaagggaggagaatagataacatccctgggtgacaggaatgtcaaagtcccaaacagcatatgacatgtgacaaatattggtgtggccttctttggaagatccaatcttccataccaggcaaagggatggaagactaaggaacaacatgagggatagccagagagggaaaaagcatcacttgttctaggaactacaaatagcttgaagaagcaaagatgtctagatgcctcccaatatgcagagtggggtgtacagaagagagtggtaagggcgctgggagagctaaggtgggcaagagagcttcctctgtcatgctaagaaagttggaatttatcttgatggtggtgaaagcagagggctatggttagattcacatttgagatttagatttttagatttaaaatgatcaccctggtgacactggcttaactcacaattttgcccaaggcctatgctaccacagtgcttctgaaactttaaagcacattagaatcacctggaggtcttgttaaaccatggattgctgggccttgaaaccccagagattctgattcagtagatcgagaatagggcctgagaatttgtatttctaacaagtttccaggtgatgctgaggctgctggcccagcgaccacatttgataatcatagccctctgataaatcctatcaaaatatcctaatggcagagcaagggaattctggtgatatcctcccctacccataacctgacagctattaggatctgcctacttgaggctaaaagcaaccaagagaggaacagctacagtgtaccacagagtccctcaacatctttgcccacgccacggtgccccagcttcttaccaagtgtgcctgattcctcttgactacctccaaggaagtggagaaagacaagttcttgcgaagccttcgtcttctctgatatgctattctatgtctatttctttggccaaaaagatggggcaatgatatcaactttgcagggagctggagcatttgctagtgacctttctatgccagaacttgctaagcatgctagctaataatgatgtagcacagggtgcggtggctcacgcctgtaatctcagcactttgggcggccgaggcgggcggatcacctgaggtcaggagttcgagaccagcctggccaacatgatgaaaccccatctctactaaaaatacaaaaattagccaggcgtggtggtgggcacctgcaatcccagctactctggaggctgagacagaatctcttgaacccaggaggtggagattgcagtgagcagagatggcaccactgcattccagcctgggcaacaaagcaagactctgtctcaaataataataataataataactaatgatgcagctttctctctctgagtatataatgcagttctgatgatgtgaggaagggcctcactgttggtgtggcagagtctgagaccatggctggcaatgaaaacactaccctttgatgcctatgggctctccctttatggtttcaaggagggcttctcaatcttggcagaattttggactggatagttctttgttgcacaggtggggggctgtcctgcacatcacaggatgtttcatccctggcctctacctactagatgccagtagaacatacccaccccacagctgcctgttgtgacaatcaaaagcatctccagatactttgcagggggaaaatgatttctccaggcctggcatatacataacagtatttaagcagctgcctagaattaattaaacacagaaggatgtctctcatccagaatgccctggaccacctctttgataggcaatcagatcccacctcctccaccctatttttgaaggccctgtgccaacaccacttcttccatgaatacttccttgattcccccatccctagctctatataaatctcccactcaacactcacacctgttagtttacattcctcttgacacttgtcatttagcatcctaagtatgtaaacatgtctctcttcacgattcacaaagtggctttggaagaactttagtaccttcccatcttctctgccatggaaagtgtacacaactgacattttctttttttttaagacagtatcttgctatgatggccgggctggaatgctgtggctattcacaggcacaatcatagctcactgcagccttgagctcccaggctcaagtgatcctcccgcctcagcctcctgagtagctgagatcacaggcatgcactaccacactcggctcacatttgacatcctctaaagcatatataaaatgtgaagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcagtaagtatccgcgccgggaacagccgcgggccgcacgccgcgggccgcacgccgcacgcctgcgcagggaggcgccgcgcacgccggggtcgctccgggtacgcgcgctggactagctcaccccgctccttctcagggcggcccggcggaagcggccttgcaactcccttctctggttctcccggttgcatttacactggcttctgctttccgaaggaaaaggggacattttgtcctgcggtgcgactgcgggtcaaggcacgggcgaaggcagggcaggctggtggaggggaccggttccgaggggtgtgcggctgtctccatgcttgtcacttctctgcgataacttgtttcagtaatattaatagatggtatctgctagtatatacatacacataatgtgtgtgtctgtgtgtatctgtatatagcgtgtgtgttgtgtgtgtgtgtttgcgcgcacgggcgcgcgcacacctaatattttcaaggctggatttttttgaacgaaatgctttcctggaacgaggtgaaactttcagagctgcagaatagctagagcagcaggggccctggcttttggaaactgacccgacctttattccagattctgccccactccgcagagctgtgtgaccttgggggattcccctaacctctctgagacgtggctttgttttctgtagggagaagataaaggtgacgcccattttgcggacctggtgtgaggattaaatgggaataacatagataaagtcttcagaacttcaaattagttcccctttcttcctttggggggtacaaagaaatatctgacccagttacgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaattacaggcgtgagccaccgcgtctggccatcagcagagtttttaatttaggagaatgacaagaggtggtacagttttttagatggtacctggtggctgttaagggctattgactgacaaacacacccaacttggcgctgccgcccaggaggtggacactgggtttctggatagatggttagcaacctctgtcaccagctgggcctctttttttctatactgaattaatcacatttgtttaacctgtctgttccatagttcccttgcacatcttgggtatttgaggagttgggtgggtggcagtggcaactggggccaccatcctgtttaattattttaaagccctgactgtcctggattgaccctaagctccccctggtctccaaaattcatcagaaactgagttcacttgaaggcctcttccccacccttttctccaccccttgcatctacttctaaagcagctgttcaacagaaacagaatgggagccacacacataattctacattttctagttaaaaagaaaaaaaaatcattttcaacaatatatttattcaacctagtacatacaaaatattatcattccaacatgtaatcagtattttaaaaatcagtaatgagaccaggcacggtggctcacgactgtaatcccaggactttgggaggccgaggcgagtggatcatctgagatcaggagttcaagaccagcctggccaacatggtgaaaccccatctctactaaaaactagctcagcatggtggtgggtgcctgtagtcccagctactcgggaggctgaggcatgagaatcacttgagcccaggaggcagaggttgcagtgagccaagattttgggggattctgtgacatacaaaaaaaatcagtaataagatatcttgcatactcttttcgtactcatatacttccagcatatctcaattcacaatttctaagtaaatgctctatctgtatttacttttataaaattcacaattaaaaatgaaggttcacatagtcaagttgttccaaacacacttaaatgtctcctaggctgggtgtggttgctcacacctgtaatcccagcactttgggaggctgagatgggcggatcacctgaggtcaggagtttgagaccagcctggccaacatggtgaaaccccgtctctactaaaaatacaaaaattagctggatgtggtggcactcacctgtaatcccagctactcaggaggctgaggcaggataattgcttgaacccgggaggtggtggaggttgcagtgagccgagatcgcaccactgccttccaacctgggcgacagagcgagactccgtctcaaaaaaaaaaaaaaggctcctaataactttattactttattatcacctcaaataattaaaattaaatgaagttgaaaatccaggtcctcagtcccattagccacatttctagtgctcagtagccacgggggctggtgaccaccacatgggacagcatatttagtacctgatcattggttctcagatctggctactcagcagaaccaagaatccacagaaacggcttttaaaagcacagccccacagcccccagccccagccttacctacctggaggctgggaaggactctgattccacgaggcagcctatgttttttgatggagggatgtgacaggggctgcatctttaacgtttcctcttaaatactggagacagcttcgaggaggagataactggatgtgtcttagtccatttgatggagggatgtgacggggctgcgtctttaacgtttcctcttaaataccggagacagcttcgagaaggagataactggatgtttcttagtccattttctgttgcttgtgacagaatacctgaaactgggcaatttatatggtaaaaaattttcttcttactgctctggaggctgagaagtccaaagtcaagtcccttcttgctggtggggactttgcagagtattgaggcggcaccgggcgtcatatggtaaggggctgagtgtgctacctcaggtgtctttttcttttcttataaagcctaactagtttcactcccatgataacccattaatctatgaatggattaatccattattgagggaagaaccttcatgacccagtcaccgcttaaaggccccacctctcaatactgccacatcgggaattaagtttcaacatgagtttcggaggtgacaaacattcaaaccatagcatgctgtctcttaaatgactcaataagctcctgtggcatccacttctgcatgccttgggcagcttttagacatctgtccattttcctagagggacaagaccaccacctgtgatcctatgaccttttggctttaggcctaacaagcaggttataccctcactcactttcaaatcatttttattgtcttgcagacaatttacacaagtttacacatagaaaaggatatgtaaatatttatacgctgccgggcgcggtggctcacgcctgtaatcccagcactttgggaggccgaggcaggtggatcacgagttcaggagatggagaccatcctggctaatacgatgaaaccccatctctactaaaaatacaaaaaattagccgggcgtggtgacgggtgcctgtagtccccactactcgggacgctgaggcaggagaatggcgtgaacccgggaggcagagcttgcagtgatccgagatcgtgccactgcactccagcctgggtgacagagcgagactgcatctcaaagaaaaaaataaataaataaataaatatttatactgcttataaactaataataaatgctatggtctgcatgtttgtgtcaccccaccattcatatgttaaaacctaatcaccaaagtgatattaggaggtggggcccttgggaggtgatgaggtatgagggtggagcccatatgattgggattagtgcccttctaaaatagcccaacggagcccagtgacaaggcatcatctatgaaccaggaaactggccctcaccagacaccaaagctgttggtgcattgatcttggatttcccaccctccaggactctaagaaacacatttctattgtttataagccacccagtggctggtattttgttataacatcccagactaagacaaataacaaatacttgtatccctgacaccaggttaagagatagaatttgtttgttcctctggaggcccttgtcttcaccccatcactgccctgtcctccctggaggaatctgccagcccgaattctgttcatcgtaccctccttttcttagagtttgacctcctctgtatctcccccaatccatgtattgcttatatacaaggtattctgctgtatctgttctgctatggcttgccccttttgttcaacactgtttttgtgcgtcatctgcattgatgcatgcagttgtcctttatttgttctcactgctggatagtatctggttgggtaaatatatcacactgtaaatcacactatccaggttcctttaggtgacatttggttgattgcagtgttctgttgttacgatggtgctgctgtgactgttcttgtgcatggacagaagttcctttcaggtgaatttctcagaatggaattgctgggcaaaggggcagccaataatcaactcatttgatgccaaaagtggtggtgccagttcatcctcccctgcgaggtatgggtcctgattcactcttcaagtgctgtggtttgacagggccgggggtgacaaggggacacctgggaaggaaagctgggctccctgctggccatccaggccagtccttaccagggggtaggcaatgattgggtcaagtggttcctgaccactgggcctgagacttcaggcccagaaactatctaatatttcctcaaatgcatcccatgagcaggcactgtgtgagtgagcacacacatctgaagcctcaagctaggcaagcctaccatgacttgtggtccaagggctcacgggtgacctggagttagagggagacatggctgccaggtggctttagaaagaacactcatcatggccaggtgcggtggcttacgcctgtaatcccagcactttgggaggccaaggtgggtggatcatgaggtcaggagtgagaccagcctgaccaacatgctgaaacctgtctctcctaaaaacacaaaaattagctgggcatggaggtgcacgcctgtaatcccagctactcaggaggctgaggcaggagaatcacttgaacccgggaggcggaggttgcaataagcctagattgtgccactgcattccagcctgggcaacagagcaagactccgtctcagaaaaaaaaaaaaaaaggaagaacactcatcctatgaccttgacctccaagctttgcctccctcaagcagaacagaatggagcctcccttaggcagaggcggaagtttgcctctcacctagttctccattcttttgttcagagcctgaataccctcaggctctgtacttggggtatttctgttctcttgttttatgctcacggttgtgaggtttgttgtgagtaccacgatcccttccttcagaggagtaaactgaggttccaaaaggtttagcagttgcccgaggaatattaaattggcaaaagcaggtagaatataaagcaaggagtatttggcaacggttcttttttatgattaaaaacagccgaagaaagacttctacttgtgcctttgaaggagtaactgcatttgaccttcccaccagtaacaaccatcaaatctctattaaattaaacacacacacacacaaacaaaaacagctattgtgaaggtatcagcgactaagacaactaaggtttgaggggccaggatcctggagagatggaaacttccctgaggtgagccccacattctcagacacttttccttggatgttttgagcactgctttaattcctgggaaaacaattccttccactgtgcacagactctggggccagacagcttgggttcaatcccagctctgccacttaatgtctgtgtatctgtgtaggcaagttaccctttggtgcgtcagtttcctcatctgtaaaacacaactatagttgatcctcattcgttaagagtctgtacttgttaatttgctcacttgctaaaatttgttaccccaaaatcagtacccctagccttttggggtcgtttcaaagatgtgtgcagagcggcaaaaaaatgtgagctcctccaggctcatgttcccagccaaggtccaacaaagtgctgccctgccttcttatttcagctgtcatagtgtaaactgtgtccttttcacagtctgattagtgccatgtttttcagatttttatgcttttttcttggttatttctctgttaaaattgtctccaagtgtagtgcaaagtttagcacgaggaggctgtgatgttccttacagagaaaatgcatgtgttagagaagctttgtcaggcatgagttaaggtgctgttgtcctgagatcaattaatttgttgttgttgttgtttgagacagggtctccctctgttgcccaggctgctggagtgcaatggtgtaatcatagctcactgcagcctctacctctctggctcaagcaatcctcccacctcggcctcctgagtagctgggactacaggtacaccccaccacacccagataatgtttttgatatttttttaggtggaattttgctcatcacccaggctggagtgcaatggtgcgatcctggctcactgcaacctccacctcccggattcaagcaattcttctgcctcagcctcctgagtagcacagattacaggcacatgtcatcacgccttgctaatttttgtgtttttagtagaggcggggtttcaccatgttggccaggctagtcttgaactcctgacctcaggtgatccacccgcctccgcctcccaaactgcagagattataggcacgaaccacaatgcccggcctcatgttttttatttttcaagttgaaatgaggtctctctatgttgcccaggttggtctcaaactcttgagctcaagtaatcctcccaccttggcctcccaaagtgcggggattacaggtgtgagctaccatgcccagccaagatcagtgttaatgaatcaactatatatattacataaggtgtctttaaacagaaataaggttatatattgatcgattggtaacaatgttgtgaccagcagcttacagggtacctagccttgtatttctcctataaataatttgctcgttgagtgtttgtggcaactttgtagcacataactaccaagaataaggactgtaataagagtacgtccctcacaggattgtaatgaagactgagtccatttacataaaggctgagagcagtgtcaagcagatggagaacactgtagaatgtgcgatagctctaacagtggttatcatggctgccctctcacttcttcagagacatgtgtttctaaggtctgcactctgccccaccctccccatccactgtcccccagcccgtttcctcctccacttacttcccagccctgtgccttctgccttctcttttctgagtttgctaagggcactgctggctcaagagcagtaactaacagtctctcgcctcttctctccatggcaaccagtgacctttggagaatgtaaaccttatcaccaatctcttaaagcccttcggtgccttcccaggatgacgtccagctgaggtccttggcaagacccagggcgccccctcctcgctccatcacctcccctgtcacctcccctgcatctccctactccagctgcaccactcttgtgccccagtggctcttgtctgattatttccttcatctccccagctggtcagcagagctggtggtaatcaactcagaccctgtcacctggatgtccagcagttagggactaaaaaaaatcaacaggtcacattctgtcctgcagatcatgataataagatctgtcagacagcagtcagcagtcagagccaaatcttctggacttcagcaggattctgcctcttgctatttcctgttgcctctcttagtgaccttttaagagcattgtggatgcctcccagcctcctgctaaccaccctgtaacctgaacagcctgcagcagccctgcccagtagaacttcctgatgtgatggaaatgctgtgtctgcaccactagccacatgtggccacaggattctcgaaactggtggtgcagttgaggagctgactttatattttatctcattaaatttaaatgtaaatagctacgtgtggcttgttggctagcctattggaaaacacgggcttagagagacacagggagaatcactgtaatgcactaaaagaaggtaaaaaaaaaaaaatcctaagaaatattcctaaaatactttaatatagggctgggtgcggtggctcacatccagcattttgggaagctgaggagggcagatcacttgaggccaggagttcaagaccagcctggccaacatggtgaaaccccgtctctactaaaaatacaaaaaatcgggtgcggtggcgggtgcctgtaatcccagctacgcgggaggctgaggcacgagaatcactcgaacccgggaggcgggggttgcagtgagccgagatcgtgccactgcactccagcctgggcgacagagcgagacttcatctcaaaaacaaaaaacaaaaaccaaaaaaaaaaacttcagcatgattatttaaccaaaatgcaggttagttgttcaccggatgcagagtccaattaacaagagcaaggcctggtaccaaaaaaagtgaatttactccgaaactagcttgggtgaggggtacaaagcatcctgcctttctttaaaagtgctgcttccccttggaagtagaaagtggacacttttataaggtaaggggggaagtgtgcaagggcaagtgggggggtccctctgctagttccgtgcatactctacaggacagttgacttggcaccttcctggttagtaataagctgtagcagtggccaagtgggcatgctttcagtatgccctcccagtgaatgaaagtcctgaggcaacccccaagggtggaagtgccaggccaccacccactggaggtgaaagttccgtgatgggtttgctttggtctgcgaatctactgtcatgtggagagatctgtgctctggaagagcatacagttagaaaagcttgccctgaagggaatgtatggtgaaggggaggtgaaaggttatatttgcatttctgaagggctaagtaggaaaccgggaaccaggggagaggagaagagaagagaggataattttttttaagaaaagcaacatattccctttttcttagaaaaaatggagcactcggttacaggcactcgaatgtagaagtagcaatatataaattatgcattaatgggttataattcactgaaaaatagtaacgtacttcttaactttggctttcagagttcgaaccaacgtggcctcaaccagatttggaatgtcaaaaagcagagtgtctatttgatgaatttgaggaaatctggaactttgggccacccagggtaagataaaacaccttccacgtcataggtatcttcctctctccttccctgcctctcccattagaacctggttttcttcctgagcagcaacaatcttaggcatctttccatgtgactgagtatccaccacattatttttaatgaaatagtattagattgcatggatgtgacataatccatttaacccatcccctactgttggacattcaggttgtttccagagtttcaatattattttatttaataccctaatagttagagcaggccatgctgctatcacaaatagacccaaatatttaatagctcaaaccaataacgtttgtgtcctcctctctgggcagtacagggttggcatacctcctgaagtgaattaggaactacactcattccagcttccagtttggtctttatctgtcagtgccttactgtcctctgcattgttgagtctcagtcaccttgtccaagttccattggccagaaagggctagaagcacagaagggctggaagtggcatttgttcctcactcacattctggtgggaagaacttagtggtgtggccttagctgactgtaagggaggctgggaaatatagtctagcgagtgccctggaaaaagccggcacggcattccccatggaaagctgtcaggcacggctacagtctaccccctgccaaccagtatctgcatggaccctccttccacactcagatgcatttacccccagccccaagagagccaaccgatgcccatgtggtcaccacagccacctccgagtccaagatttccaggtgacatgcagtctcctctctcccagctttaggaatggcttcttctgatctacacacagacacagacacacacacacagacacacacacacacacacacacacacacacgatggagaggggcaggataactgcaactgtaactccattcagaaaagaggcacagcactagctgcccgcagcactggagccctgctgggcagcactggatcaacctctgccctggcagaggagcatgttcctccacaaatccctgcttcagcctctcgagaggctcctccttgtctgttattttccttggccacaaggcaggcaggcagtgggaagtgtgccctcctccggggcaaggagccttcacagcccacttcctgctaataacagtttggggttccacagggtgttttaagactccagtcagctattttaggccagactcatttctctctctctctctctttttttttcttttatgaaatcacaccctgagacccaggctggagtgcagtggtgcgatctcggctcactgcagcctccgcctcccgggttcaagcaatcctcctgcctcagcctcctgagtagctgggactataggcgtgcagtgccacacctagctaatttttgtatttttagtaaagacggggtttcaccatgttggccaggctggtcttgaactcctgacctcagatgatccgcccgcctcggcctcccaaagtgctgggattactggcatgagccactgcgcccagcccagactcatttttctttgagagtaggcttttcccaaaagtaggcttctgagctattcactttcaggcagtcccatgtgccaggaaccacatccaaatttcctccgtggatgggagtctcaggctgccttatctccttgcatgtccccatgcccagctgtctcagcctaagggcaggtaccttgaagtcaagttaaacaataagattggagaccagcaatgccctcagcctggtttttgcagcaggactgagtcccttgttttggctcaatgggaagtctttgctgttcaaagccttagcttctctggctgagtgcggtggctcacgcctgtcatcctagctctttgggaggccgaggtgagcagatcactgaggccaggagttcaagaccagcctggccaacatggtgaaaccctgtctctactaaaaatacaaaaagttagccaggcgtggtggcaggcacctgtaatcccagctactcgggagcctgaggcaggagaatcgcttaaacccaggagatggaggctgcagtgagctgagatcatgccattgcactccagcctgggtaacgagcgaaattccatctctaaaaaaaagaaaaaaaaaaggccttagattctccctttgactttccacgtttgtgcagccttttatctccaatgctccatttcattccatctcctggcttattcttttcttgtcacatctactaaaagcaacaagaagccaccggtattcaggaacattctacctgtccccagagctatatgctcagtaggcatacagttggccctccaggttatctgagactcagatttccagagggctttgcatggctcacaaggtctgaagaacctctgagcctcccgcctgcggtgtctgttcattgactttgccacagtctcaaagaggcactgcatgctgcatgtttgaggtttttgctttggtggcatccatttccagcctcggcttccggcattcctcccccagcagactctctgctgctttccccttactccttctggcagttctgggaggttgcatagggcccttgcaggatgccccaagtccagctgcctctggcctctgggaagcacacccttgacctgccatgtgtaggaagacagcccgcttctgccagggcccaactctgccggcaggtagcaccttccaacctcttcactttggactttataactgtcaggtataaagtcggttgtgtccttacgtttctcaaattcttcaagacacgtcaaccagcctctcctacgcattctctccagctcagtctcaaaacacaccctttctctccagctcactctcaaaacacaccctatcaggccaaccactctttttaaaggacagctcctcaccaatccagtcaggtagccttccccacattgtatcctggaagtgggtgatggactgggtggggaagagggtcatatggcaaatctgtatgtcttacagtaattgtctagcagcccctggtgtcttactttaggccccctggaaactttcagatagtggagttgtctgatacatatcttataacctacagatattaatatatcctcacaggggcacaaaagctcttacaaggatgtttattataataatatttttattgttataatttacatgccataaaactaaccattttaaaatgtataatgcaagggtttttagtatattcacaagattgtgcagctgtcactactaattccagaacattttcattattccagaaggaaaccctattcatattagcaatcactcccccattccgcctttccctaaaacccagcaatcactaatctactttctgtctctgtggatttaaagtaattttaaatttgaaaaatagtatctataaggaaatgtatctagtcacaagcatacagcttgatgaatttgtaaaaattgaacagtcctatgaacataccctgtaagctcaagacatagaatgttaccagcccctgcaagcaagctgcctgctcacttctagtcattaacccctccctcttttccttctagtcattaacccttcagagtaactattctgattaccaatagcatagattagttctgcctgttgttttactttatataaactgtctcattaagtataaacatgtttgtgtatacttgtgtatttctttctatcacaatgatgtttgtgagattcatccatgctgttcctatagacaattctattttgcagcgtagtattccattgcatgactataccacaatttatctgtgatattacaaaggaatacttgggcagtttccagtttggggctataggatagttgtgatacaaatattttagtatagtacatgtcttttggtgaacctgggtacacatttctgttgtgtataccccttaagagtggagctgatgatcctggctaacaaggtgaaaccccgtctctactaaaaatacaaaaaattagccgggcgtggtagcgggcgcctgtagtcccagctactcgggaggctgaggcaggagaatggcgtgaacccgggaggcggagcttgcttgcagtgagccgagatcgcgccactgcactccagcctgggcgacagagcgagactccgtctcaaaaaaaaaaaaaaaaaaagagtggagctgatgggtcatagcatgtaaatgcattcaactttagtagatactgtccaacagttttccaaagtgattgtccaacttacttgcctatcagcagtatctgaaaagtctagttgcttcttttcttggccaactctttttttttttttgagatggagttttgctcttgttgcccaggctggagcgcaatggcacgtcctctgctcactgcaacctccgcctcctgggttcaagcaattctcctgcctcagcctcccgagtagctgggattacaggcatgcgccactatgcccggctaattttgtatttttagtagagacagggtttctccatgttggtcaagctggtctcgaactcctaacctcaggtgatccgcccgcctcggcctcccgaagtgctgggattacaggcatgagccaccgcgccaggccggccaactcttttttattttattttattttactttaaagacagggtttcactttgtcacccaggatggaatgcaatggcacgatcacagctcactgcagccttgacctccctggctcgggtgatccctcccacctcaggctcctgaggagctagaactacaggcatgggccatgcccagctaattttttaatttttggtagagacggggtctctgttgtctcagattcctgggttcaagtgatccttctcccttggcctcccaaagttctggtattacaggcatgagccactgcacccagcccatggccagctcttgatacgatctgtctctttcttttctttttttttttttaatttgagaagtgttaaataatctttctttgatattatacataaaccacaccaaaatgtctttcagtaagtaaaatgaaccattttagatacagaaaattctaattagattggcatagttaaggccaaaaatataaagttgacattgctaccttatcttcagcccttgcctttaagaggcaaatgaacacaaaatacaggtgaatcttgcttggttctgagacagtgaaggactttcccccagtatttaaatatatttacataaccagttacataaatctaaatattaaaaaaatctccaatagattttagatggcattcaccatctttgtgaaaagttgaacattactaatgaaatctgatcatatctttagaaggataaacagtgatagcatttactgaatcagaataactgttttttggggttttctttgagacggagttttgctcttgttgcccaggctggagtgcagtggtgccacctcagctcactgcaacctccgccccctggattcaagagattatcctgcctcagcctcccgagtagctgggattacaggctcgccccaccatgcccagctaatttttgtatttttagtagaggcgaggtttcaccatgtcagccaggctggtcttgaactcctgacctcaggtgatccacccgcctcagcctcccaaaatgctaggattacaggcgtgagccaccaggcccagcctattttttttttttttctttttttgagacggagtctcactctgtcacccaggctggagtgcagtggcacaatgtcagctcattgcaacctccacctccggggtttcagtgattctcctgtctcagcctcccaagtagctgggaactacaggcgtgcaccacaagcccagctaatttttgtatttttagtagagacagggttttgccatattggcctggctagtttcaaactcctgacctcaggtgagccacctacctcggcctccgaaagtcctgggattacagacgtgagccactgcactgcctggcccagaaaggactattaattgtagttgcctctgggaatgggggctgcctgcttctttctgtaaccccttctgtgctgtttaaatttttttttttttttttttttttgagacagagtctcgctctgtcgcccaggctggagtgcagtggcgcaatctcggctcactgcaagctccgcctcccaggttcacgccattctcctgcctcagcctcctgagtagctgggactacaggcacccgtcaccacgcccggctaattttttgtattttcagtagagacggggtttcaccatgttagccaggatggtctcgatctcctgaccgtgttatctgcctgcctcggcctcccagagtgctgggattacaggcatgagctaccacgcccggcctttaaatttttactttgggccgggcacggtgccttacgcctgtaatcctaacatttcgagaagctgaggcacgtggtggatcacttgatgtcacgagttcagaccagccactgcactccagcctgggtgacagagtgagactctgtctcaaaaaaaaaaaaaaaagaaagaaaaacttttactttttacatgttattttcatcaatttaatgaatttaaataacaaatgtataaatttgatattaataaaatggaagcatttggtaatcatgttttgggttttgtgcttcctctgcagctctctagatgagaccacctatgaaagactagcagaggaaacgctggactctttagcagagttttttgaagaccttgcagacaagccatacacgtttgaggactatgatgtctcctttggggtacctcttgacttcttttatttttctgtttccccctctaagaattttagttcactaaaatgaagaatttccctccagcagagctaagcatcaagtagcatgtagttgtaggtaggattaaaagactagggttccgggaggtgaaggttgcagtgagccaaaatcacgccactgcactccagcctgggtgacagagcgagactctgtcatagatggatggatggatggatggatggatggatggatggatagatagatagatagatagatagatagctggatagatagataagatagataagacaagactaggcttcaagctgcagtccagctctaccaggcttgttgtgactctgggcaagtcactcagcctctctgagcctcattttccagcttcagtggatacccatgaaggcaaatcagagaggggcctgagtgtgtatttgtccagcaggcagatggagggaacaacaaactagacccgtagttcttcagtagggataagataactgcccaaaagttatttagattacaaagacttgagccctgctcctgtgagacagtgatggggtaggtcgggtgcattcctgggaagcatatttttgaaaagctcacctgggattctaatgtgtatccctaggtcttattcctagagattttgattacttggtctggggtgtggcatgacctgggcagggcactgggatttttaagctccacagatgattccaatatgcagctagtatgagaacttgtttttttttgaaggagtctcactctgtcacccaggctggagtgcagtggcgcaatctcggctcactgctccgcttcctgggttcaagcagttctcctgcctcagcctcccgagtagctgggattataggcatctgccaccatgcccagctaatttttgcattttagtaaagacggggtttcaccatgttggttaggctggtctcgatctcctgacctcaaatggtccacccccatcagccttccaaagttttgggataacaggcgtgagccaccaggtccggcctggtgtgagaacttctgagttggatgaaacattagccccagatcctagaagccagggaagtgctggtctttatcgactggccaccaggtggcagatttgggcaagggtctgcctttgggtttagaattattgcttaggccttaaagtagttcttttttgccagtgggagaaaatccctcaaagatggttttctgggttggttggtttgtttgtctgtttgtttgttttttgagacagagtctccctctgttattcagcctggagtgcagtggcatgatctcactgcaacctctgcctctcgggttcaagcagttctcctgcctcaacctcccaagtagctggaattataggcacacgcccccacacccagctaatttttgtatttttagtagagacagtgtttcaccacgttggccaggctggttttgaactcctgaactcaagtaatcctcccacctcagcctcccaaagtgctaggattacaggtgtgagccaccgcgcctggctcctcaaagatgttaatcctcttgatggcaattgactaataccagaaaatgtcacgaagcgtgcattttggattcaatcatggaattgttgaggacaatcagccatcagactaaagcgatagaaatagtattggaaattgcagcgggagcactgaatggagaaggcactccacataatggaggaggcaaccaagtcttagagaaggtatcaagcctgactataaggacagtgagggaattgaaaaaacaaaaaaggagcaatggagcagggaaggattgaatgcctttcaagtagattcagtaattgctgttagcagcaaaaaatgcagtagtgcctgggcagggctttaaagtgcttgcacaggcagccctagagggccgggctgcttgggaactcttacaaactgacctaccaacttgagcatccacagcctgatcagaggtgggggagttaagggccttctctcccctagcctctactagagcctgtaactgcagggaaaccaagttgcaggctaaactctgcccacacatgcagacattgattagcaagctacaaaaacagtcatgaaacctgtttttataggattagtgaagccccagtttgaccagagtactttgcatgaatgttttgttagaagcaaatgtgccaatattctagcagctgcgtttggtttacttcttcttcttctttttttttttttttgagttgaagcctagctctgtcacccaggctggagtgcagttgtgtgatctcagctcactgcaacctctgcctcccaggttcaagcgattctcccgcctcaacctcctgagtagctgggattacagacatgtaccacaatacagggctaagttttgtatttttagtagaaatggggtttcaccatgttggccaggctggtctcaaactcctgatctcaagtgatccacccgcctcagcctcctaaagtgctgggttaacaggcatgagccacggcacctggcaaaagtcatcttttggtttacttctattgaactgaaaaagtcacaaatatatttatatttaattaaatatatttatataaaaatatggtatttagtattattatttttagagacagggcctcgctctgtcacccaggctggagtgcagtggcacaatcatagctcactgcagcctcaagcttctgggctcaagtgatcgttccacctcagcctccctagtagctgggactacaggcacatgccaccatactcggctaattattttattttatttatgggtctcgctatgtttcccaggctggtctcaaactcctggcctcaagcgattctctcacctcggcctcccaaagcaccgggattacaggtgtgtgccagcacacccagccacaaatctataaatttagaaaggaggactatttctaaagagggtcccactacctgtaggcaggaagcagagcctctggccataactgaaaaacaagcacttccaagaaggggcaaagggaacatgaatttatgctgagaggcgtagctaagcatacatattcaacagattatgggaggatctatgaatattcacaaagggaggatctatgaatatgcacacatgtggagtaagctaacgtgtgcagcatgtctcccatgttcaccttaggcagaaacttaacactaacatgtattacagggcaacaaaatgagactgcatatctacataacctagctatttggtaggctgaagcaggagcatcacttaagactgggagttcgaggcagctgtgagccatgatcgcaccactgttctccagccaggatgacagggcaagaccctgtcttagaccactctgtggtcagtggttatcaggaaggaatgctagtcagttgtgctgaaaccactaaaaaggaagggcagaattaggtgatgagttgataccagtggtgaagtgagtcttttttttttttttctttttgagatggagtcttgctctgttgcccaggctggagtgtagtggtgtgatctcagctcatcgcaacctccacctcctgggttcaagtgattctcttgcctcagcctcccgagtagctgggattacaggcgcctgccaccacgcctggctaatttttttatatttttagtagagactgggttttgccatgttgtcaggctagtcttgaactcctgacctcaggtaatccaactgctttggcctcccaaagtgctgggattacaggcagctccaaagtgctgggattacaggcatgagccaccatgcatggcctgaaataatttttttgaaagggctagtttctatttagcccttaggggaaaaaaaactaatggcagttagggagggaatagaacgagtcctgtttgaactcctttcccatcatggccaaaacttaaaattttttttagatatctctgggctccccttggccaaaagatagtttgttgagtcagttgggagcttagaattttgtttttatttctcacatcattgaatcaatttgaaccaggcgacaaaaccttctgctcccagtagtgggtcagagaaccttcctgattcctgccctgagattgtctctctgaagacaacattaggctagtaggctttccagattctgtaacccattctttcaaaggaagagatgcctatatttttctagccaattcatataccttgagtatcactcaagggcaaaattatttctaacaaatcatttactaattagcaaatgcttaagtgtagatttagaaagctaaagctatacagtggctgccatctatagtttggacttgtgattaactacattgaaatgctaactctgtaccctagagtatgaattcctgattagagtccttcaggtgctaactaatttatgtatttcatgtttgataatattattacttgagctttgtgggagagcagtcttttcctcccctgagatatagctagaagttacctcctttgtgaagccttcctagatactccaagcagacacggtccttcctttctcccttgcccagcactctgaggttgactctgtggagcactgatccctctgtgttataattgtctatttacacgtcagctaccacctataacacactgagttcctcaacagcagggacactgtccattctttgatcccagtgtctggaacagtgccaagtacatagtagggacttaataaatattgattcatatgtaaatgagacttttccaaaacatgctttcgttgatgcctctcagcatttatacaccttttaccaactcgctactggccacatagacaaatgaaagcagtaatccagatacacccaagaggacatctgttcttttttctctctgtggagtgggagacttaagtggcttcttaactggtgtgtcgtctgatcaagtggtccaggtaacaggtggatgccaatgtctggcccaggcatcaccccttactggcactggtcattacagaagacactctaccagagctgaaaggacctcttgtcactaggcagctgtggagtccgctctacttgacctagtaaaatctgcctggagactgttagagtcaccccactacctgaagttacctccaggctgacctcttttttttcccaggtggagtcctggcatcttagatattttaataaggatttgcttgttgacatgttctttattcactaaggtgtcagcatattactgtcttagaactgagggttcttcatcttttttggatcaggacctccctctaagaatctgatgactgctctggtccctctcccaataaaaacttccatactcacctgttaaaaaaaaaaaaaactttaaacaaattaacagagttttattcagcaaagaatgattcataaatcgggaaggctgcaaccagaataggttcagagagactccacggtgtgccacgtggttggagaggatttaggatttatgcacagaaaaaggaaagtgacatgcagaaaatgaaagtgagggcctggtgctggtgcggtgcctcacgcctgtaatcccagcactttgggaggccgaggcgggcagatcatgaggtcaggagatcgagaccatcctggctaacacggtgaaaccctgtctctactaaaaatgcaaaaacttagccgggcgtggtggcaggcacctgtagtcccagctacttgggaggctgaggcaggagaatggtgtgaacctgggaggcggagcttgcagtgagccaggatcccgccactgcactccagcctgggcgacagagcgagacttcatcttaaaaaaaaagaaaagaaaaaggaaaatgaaagtgaggtacagaaacagccaggttggttacagcttggtgtttgccttaaacttggtttgaacagttggccgcctttgattagccaaaactcggtgattggtacaagagtagattgcagttcactatgtacagagaagcccttagatccgaactcaaaataggtaaggaggcagttttagctacacttaagttaacatactcaggagtaccattccagcttcaagctggaagtgtctgcagccccctgagaccacttaatcccaagttaaaaacccctgctcagaggcagcatctttttttttttttttttttttttttttgagagagatctcactctgtcacccaggctggagtgcagtggcacgatctcagctcactgcaaccaccacctcctgggctcaagggattctcttgcctcagtctcccgagtaactgggattacaggcgcgtgccactatgtccagctaatttttttttttgtatttttagtagagatggggtttcaccatgttggcctggctggtcttgaactcttgacctcaagtgatccactggcctcagcctcccaaagtgctggcattagaggtgtgagtcactgttcctggcccagtgaggcaccatctcattggatatggagacaaaggatctggcttagcatcctggatttgtattttctttccaagagtccttaagtgatatctaacttttgcgagctgcagtttcctcagctatgagatgagtgacattaacctcctctcttcagatttataagaggatcaattaaaatggcataggtaaaagtgcatcctagcaagttggtatctactttagaaatgaaggaggtcatatgtatgtgaagtctccagacccaacatgccatcttatatgtgtctatttctacaagtgagctagtgacaacagtaattgctatttttgctcctacatgggtagggctgatcttgactaggaggagtcaataagactcaccagccgggcgtggtggctcacgcctgtaatcccagcactttgggaggccaaggcgggcggatcacgaggtcaggagatcgagaccattctggctaacacggtgaaaccccgtctctactaaaaaaatacaaaaaaattagctgggcgtggtggtgggcgcctgtagtcccagctactcgggaagctgaggcaggagaatggcgtgaacccgggaggcagagcttgcagtgaaccaagatcgagccactgcactctagcctgggtgacagagcgagactccatctcaaaaaaaaaaaaaagactcaccagctgtggccactgtctgtgctaattggctagtgcctgcatctcagaaactgctacatattttgactattccccctgcacttaagggcatgcacactcccaaaatagactcagattgtctaaggaataatgatgatgatgaagagaaagccctctttatctggtctatttgtagtcagttccaaaagcattaagaatttctgctgaactaatgcagctagtttctttcctgtcaccactttccttccaaaatagtttcaagatctgtgggggaaaaaatctatttacagtgaacagactggtgggaggaagttgagcattggggttttctgccctgtgtaaccttgccctaagttgggcagatggtatcacactacctggacatcatctgctcattcactatttgaccagttggtcattcattcacaaatgtcctttttgcaggagggatggaggtgctagacctgcagatgctagcatgaaaagacagatctcctgctgctaaggtgcttaaagtagtggaggtcaggggacaagcaagcagtcaggcagctctgaatgcagaggcaggaagcaccacgaggcaatgggacccacagaggggtagcagggtagaggtgagtgggtctcatgtggggagggaggaagttgactgcagagaaggtgccagggggtgaaaatagcttgagagctgtggagctagaagggctctcacatttgcttattaatatgccctttgaaaaagagtggcctgatacctggagtcactcaaaagatttccaattccgataggaaaaagtcaattttggcttcagtggttgcatgtgcaccccctgatttgctgtatgctgaggcattgtggtgatggacgcaagtgcggagaccttgagcacgcatctgcccctagttcttgccctgagtcctcgaaggaggcaggagagacatcaaggcagacaggcgccgctcatcagtgatgagaccagacctggaactcgcgtcttatactcagtcctctgccctttctgctggattgtggccccccagtatagggtgcaacacacaactggagcatttaagggccacaaagagaacaaattaccaatgattgtgtgttgattctttgagctctttttttttattattatactttaagtgttagggtacatgtgcacaatgtgcaggttagttacatatgtatacatgtgccatgctggtgtgctgcacccattaactcgtcatttagcattaggtatagctcctaaagctatccctccccccttccccctccctccaccccacaacagtccccagagtgtgatgttccccttcctgtgaccatgtgttctcattgttcagttcccacctatgagtgagaatatgcagtgtttgattttttgttcttgcgatagtttactgagaatgatgatttccagtttcatccatgtccctacaaaggacatgaactcatcattttttattgctgcatagtattccatggtgtatatgtgccacattttcttaatccagtctatcattgttggatgagctctttatctcatggaaaaataatttataaaactctgtatgagaggagtgggaaatagtattaacgggtgcggggtttctttttgggacaatggaaatagctggaattagatagtggtgatgtttgcacactttgtgaaatactaaaaactcctgaattatacagttttaagaaacttttatttatttgtttttgagagaagttctctgtgtcacccaggctgaagtgtggtggcgtgatcaccggttattgcagcctcaatctctgaggctcaagcgattctcccacctcagcctaccaagtagatgtgactataggtgcgcaccaccacacccagtgaatttgtaattttttgtaaaaacaaggttttaccatgttgcccagtctggtcttgaactcctgggcccaagcgatcctccctccttgggctcccgaagtgccaggatacaagcatgagtcaccacatgcagcctcagttttaagaaacttttaaataaatgaaatatagtcataccaaaacagtaaaaatgggtttcaggaaaaaaaatgtttttttaaacaaacttacgtattgtataatcccagcccttttaaaaaatgctttcaaaaactggcagtcaactcataaaaggacaaatacttatgattccactgatgaagtagtcaaaagtagtcaaaaatcacagaaacaccaccataaatgtataatttttattttcaattaaaaaaacatcttttttttagtcaaaatcatagaaatagaaagtagacaggtggttactaagggctatgggatggggaaattagtgtctaatgggcatagagtttcagtgttacaaggtgaaaagttctagagttatgctgcccagcagtgtgaatatactttattgttctgtacacttaacatggttaatatggtaaatttagcgttatgtgctttttactatagtaaaattaaaaaaaaaaaaaatggggccgagtgcagtagctcacacctgtaacataatcccagcactttgggaggccgaggtaggaggatcacttgaggccagaagtttgaaaccagcctggtcaatatagcgagacctcatctctacaaaagaaaaatgttaaaattagacaggtgtggtgtctgtagtcccagctctctggaggcagggactgagtcagaggatcacttgagcataggggtttgaggctgcagtgagccatgatcctgccactgctgcagcctgagcaacagagcaagaccctgttgtaaaaacaaacaaacaaaaactggcagctgatacctgagagtgaatatcttttatcgctggttaatgggattgagagaatgcttcatcttatagaaagaacagtgtctttggacccacagagacctggatttaagattagctctgccaattactgagtactctttactatgaacctctgttttcctcatctgtgaaactggaataatgaatcctaccgccaacaattgtagtcaagttggaaacaatttacacaaagtgccaaacaccaagcctggcacagtaggaaccgagtaaatagtggttaatatttttatcagtgtctgcattgctgacgtctccatcatttctatacatttgtttttgaatcagaaaaagatgttattttaaaaaaataacccagtagtgccccttgtcccattcctatcagttatattattattgttactaccctctggaatttcaataactctttgttttttgggttttttgttttgttttgctttgcttttgagacaggatctctgtcgcccagtctggtgtgtagtggtgtgatctcagctcactgcagcctcaacctcctgggctcaggtgatcctcccacttcagcctcccaagtagctgggaccacaggcgcatgccaccacacttggctaatttttgcatttttagtagagacagggttttgccatattgcctaggccggtctggaacttctgggctcaagccatctgcctgcctcggcctcccaaagtgctggaatttcaggcatgagccatgcctggcctaaatagctctctgtgtttgcaaaagtgtgttataagaatcattcagagcctctcgattggatggaggctctagaatgcacagaaaaaggctgccaccgtgtatctctgcaagtcatgcacaagatggggaacagcaggcttccccctgcttaccagttcaaatacagagaactagccctgtagctgtttctttcatatctcacccattctaaagagaccacaggccttagaagtaaaggactcttttgttgaaagagtgttttcaaatttaaatgagcatttattggtcaaagatgcaccaactagtcttttgaagaattcaaggctctttagagaaaaataaagccttggaggagtatctgagaagcttgttagatgcgtgggaagagtctggaaataaaaaacttcatctggagtttctgccttctaccaacagagctgaagctaatgctctcctaagacaagcaaagcagatggtttgcatacttccttaccttccttttacttcctctgtaatagacttgtcatgtctgatgtttgagttgacgtggtactctaatagagttagagtctgcattttttttatgtcctctagtatgttctggttgatggttgagggcaacaaaccagcagtcccagatgccagcaccaagacctgagacaggtcacttaactctccgagcttcaccaccattctcaccttgcagacctcacagggaacagggaaagctctatgagatacaacatcattatgattaatcctattctgattctgaaagcaaagctcttcctacacaaactcctatttctaaatactaaaagacatttctttatggtgtattttgtgtacttgtagaaatggaaagtgttgagataaaacatgaagcaatgatgacaaagtgctaactttttcttgttttaatttctttatgctttttttccacctaatcccctagagtggtgtcttaactgtcaaactgggtggagatctaggaacctatgtgatcaacaagcagacgccaaacaagcaaatctggctatcttctccatccaggtatgtaggtatgttcagaagtcaacatatgtaattcttaaagacttccgaaatgtgacattgtggaccatttaagaaatgtcggctgagcacagtggctgacacctgtaatcccaacactttgagaggctgaggtaggaggatcacttgaggacaggagttcagaaccatcctgggcaacatagtgagtccctgtctctgtaaagaaaataaaaataaagtcacagctgggtgcaggcttacacctgtaatcccagcactttgggaggccaaggcctgtggatcacttgagctcaggagtttgagaccagcctgggcaatgtcacaaagccccacctctactaaaaatataaaaattagccaggtgtggtggcacacgcctatagtcccaactacttggaaggctgaggttgagcctcagcctgagcccaggaggtggaggttgcagtgagccaagatcgcgccactgcactccagcctgggcaacagggccagaccctgtcccaaaaaaaaaaaaaaagtcatcgtcttatgttagcatccttgtaagtgagcctttcctgatattttgcagcctgtctcattctcagtagaaaagtttactctagttacataacttctccctgctgacaatttggatactgtaagcaggcatcaggatattaagatctgaagtgagtagcttataacttttccaaatccagcctagacagttttcctctattaaattattgccctgactttaaaagaagctacttttgaccttgtagcgtttgaacaagttgcactttgtcttcaaagcaagttaaagtttgacctctacttgttttgagcctctcaggtaaagggttatttgaattccctttgcaggttggggttgtgtaccctgtggaggtggtagagtgttatatattgctgctccagggcatttaatccctcctgccttttccattgatgtgctttcaatctagaggaataaaagattgtgttggagacacaatgtggcctgcatagcatctgaaagcctgagaacatgcagggagagacatccctcatccctcagcagcctggctgctgttgaagtggttgtaagaaagtaaaagagaaatgcccacaaaacgttctcagatccagtcattcattagcacttccaaagagagcatgttgactgtgaattgggaaagggccagataaaactagcatagaattctttgaaagactaacggtatttgcattttttaaaaattataaccttactctaccccctaacattgacatcatttttaggtaattaatattttcccatttattattctgtgatctctaatgctttgttcagaataaatagtgtgtttcctttccccacactttcatccaagaagtgtgctagagttcaacaaaaacagcactagaaatcactgtcattctaggaaggccctaattcacagattgtattggtttttagacccagttagtgtgctggaggttggaggattttaacctctgtgggccaactagcctctgtggcctcagtcattcttcctgaccctggctgtgcttgagcctgtgtgttcttatccttcatctccgggggaacgaagtggatcagctcggtccagcgatcacttttggggatcagtggctttgtagatatcgggcaggcacttaccccaaaagaactttccccatatctgaagactgaaaacgtccatatcgtatttggacacactgcccagcaatacgctctagctgtgttcagaagcatgggaatttggaaagatctgctgagcatgccgtttactgtcacagatactatcttcctcaaaaaaaaaaaatatatatatatatatgggggacggggcaggttgagactgggtgagactgaagaggtgccttggccagagcaggccacacccagagaccacaggctccccggtccacctcaggcccctccccttcctgcgccgtttccggcagatccagagtggccaccgccggatgggagtcgggggaagggaggcagagaagcgggccctgaggacaagctctcagtgcttctgtgggaagtggcggcaagacggcagctcccagcgggggatggaggccgagtcagtctgctggtcactggaggccaggatgctgcctaacacagccgtcccgctccgggcctcaccaccagggcggctctccccactcccggcctgctgcccacacagactgcggggttccgggggagcaggacccaggccgttctgcgcctgtcttcttggaaggagcaggccggagcgcgggagcgccgtgtagctgtacctgcgaaggcacaggattccgcgggaagatcccgcagtttcgggccgtcgtcattgtttttatacctgtggcaaatggcatgaccagacacacggttatgtctggagaaacccctgtagaggagcaggaggttgtggacatgctgtggcccggacagtggctgccgagcagttggagcctgcacccgcccaacttggctaaagaagtccccatactctctgtggaaaagatttccagaagctgttgtgtcaatatcaaagcctcaaaacaacaacaacaacaacaaaaacatgaaattatcaacaataaagatcatccttgagtctgctttgaaaagtagggtgaaattctgcagaggcattcaactggcaagataccaccctcatagccagatctgcaggtctcagccatcatgccagggaaaatgctccattcaccactcctcagcttctgcttctggtttcagaggtctctgtattggaggggctttaaagcaagaagggtctttacccacttactcttattcacagatgtgaatatgcaggtccagtggggaaagtgacatgtcctaagtcagaatagagtcaacaagaaaacagggcccaaaatgacttagcctctagtgtataatgggcattgatgagctactggaaatacagagatgaagaaaacacagtcccatcttcaaggagctcaatctagcaagggagacagactctttgtaggtgggaccgggcttccctgcagcagaaggaagcttgaaattggtaacgagcctcagaagggacagaggcaggccaccatgctaccctgagaggatcgcatgtggacacggggctatgacctggccctgctttgacccactagctgtgctgtagggccaggtggagcctggagtggcctgtgctaaggggctactatgagctctttccactcccccaaggcattgcataaataatgtcactttctgtttgcacagcaaaatcagggacacaattttctagaacatggggtgcctcccctccccccagcccaacagaagttctacaatgactgatgggcccttgtttttgtttgagacggaacaccccacagggttccgagtggtgatttgtggcccacaggccactggcaagtggaggcagagctgcagagccctcgggagccacagagggcctgctggccgccacgacatgccaactcagctgctgctggccctcctgtgggcggcagtgctagtgatgtgcagaatcttaggactagtgccaaggaacctataaataccctgggtgacccaggcgtgcactgctgtggtggccttcacagtcagaagatgacaagctgagaaggggagaatcggcccaaggtgagatccacagaaaggccagggccaagatgcggccagcacctcaggctggtggtggtcttacgttgaccatgccagaggccagtccttgattgctccaaaccctctgttcgagggttccaaatgaaatgagcaggtcctcgtgtcaggacctaggttagtttctgaaaaagcatgaaaagcaggcctcctgaacttccccgagtgactgatgcaaagtgcgtcctgcatgcttcacagcaccatggagaggatcttcaggggcaaactgcagactatctgaatgacggcactgaccatcagcaaaccgcagagctgcctgaccaagaaattgcgagacagaagcaatgcttgcaggcgaagaagaaggggccagacacagtggctcacgcctgtaatcccagcactttgggaggccaaggcaggcggatcacttgaggtcaggagtttgagaccagcctgggcaacatagtgaaaccctgtctctactaaaaatacaaaaaattcgccaggcatggtggcaggcacctgtaatcccagctgcttgggagactgagacaggagaattgcttgaacccaggaggcgaaggttgtaacgaactgaaatcgtgccacagcactccatcctgggcgacagagtgagactgtctcaaaaaaaggaggagaagaaggaaaggccaaggcaggaatgaaacaggccatgaatgttggagtgaagcaactggcctcctcgtgctaagcggctactgtgagttctttccactcccccaagacattgcataaataatgtcactttctgacactcaccccgctgaatgtcctgcctctgctcaagggtggtatgatggggacttggcagtggaggggaacagggaaaccagacatggtggtctccccgcttcctggctacaagtccctctgaagaaatccaaaggagtaaagagcttggagagtaggcctctgtagggtgcaagggcacagctggagacggagctcctgaggctgcagctgatgctgcccgctctgcctgaactgcaccaaaaacgtgatgaggccatagcgggagtccacggaggaggatgcctactgcccgacctctagcagagactaagcaaggtgcatgaaaacttgaaccacatgtgtcacacccatgaccactacatgaagatggcccaaaacctggcccaggaattgaagaaagactcttccaatttgctgtaagaaaatggcccagggggcaagcacggtagctcacacctgtaatcctagcactttgggaagctgacgcaggcagatggcttgagctcaggagttccagaccagcctgggcaacatggtgaaaccccgtctctaccaaaaatacaaaaattagccgggtgtggtgatgcatgcctgtggtcccagctactcaggaggctgaggtggaaggattgcctgagtctgtggggcagaggttgcagtgagctgagatcacaccactgcactccagcctgggtgacacagtgagaccccatctcaaaaaaaaaaaaaagaaagaaaacggcccaggaaggctggagggccgccgtgtccattgagagagtgctccaggcactccaaaaagaaaatgaccacaatgggaagaaaccagctgaccatgagaccaagttccaaccttttacaagtggcctgtggctcctggcgccccgcccacagctgacaggggctcagaagtgctagggggaccatgggccaccagggccaccaggagggaggcaggtaacgatgcgagggcttggatgcagaacaccagctggtttgattctgttttccctgtacctgggtcctgaatgcccagaggctcagggaaacaccagccagtgctgctgcctttaaagcacttttgactgatctcttgttaatttagcaactgttattggttgatgctgcagttgctcttattgaagtttgattgatagcattaggatggtaaggcactatttttcaaataaaggttgtttaatataaaaaaaattttgtttttttttctctcagcctttcacattggttcaaaatatctttcatctggctgcatttctgatttttgttttgttttttttttcttaattttatttatttttaattaaaaataattttttttgtcaacatggggtctcactttgttgcctaggttggtctgaaactcgtggcttcaagcaatcctcccacatcagcctcccaaagtgctggggttatgggtgtgagccactgcagcagcctgttttttttgtttgtttgttttttttaatttgacaagttttcaggtcctgtgaaatcagcagtcttacctcccaccttgcgcaccctgaggaggttgcagaataaaggagaattctagggacacgtgggcatcagtgcctgtgctcagagcacctcaggcagtgtggaggggtctagaggttactcaggctctgcctggcaacccgatagcagtatcagagtatagggccaaggggacggtccttgggcttggtgtggtttattagtccttttcctgtgaccctgatggtttggttcactcatttttatctccatactgggaacaggttcaagccccagcatttggttgataatgcaggaatccttgatacttttattgcccaagcttcccttcctggtgacctcatcctagcctcagtctttggaaaagccctccttgagtgctcaggcagactcaggtgccctttcttctgggctcccatgcactctgttcttacctccatcagggtgccacatgcactagtgttatctgctgccgtggccaatcatccatgaggccatgaggaagtggaatgtacatctggtataagaagacatggcagaagccagcctccgatctgtccacacgaatacagcattcccaaagcaacgtgcatgtgccattattcactggatgagcttgaggtggatgaactagcccaccaggctctcaatgtcatgaatttaacactgaattaagaaaaatatgttttaaaaataatagtttaggtgattgctggggtgctaggagaggaaggaatggggaataactgtttaatgggtatagttggccttgtgtatctgtgggttccacatctgattcaaccaaccgtggatcaaaatatttgaaaataaaaaacaaaacaaaaatgatacaaataaaaaccaatataacaactattaacagcatttacattgtactaggcattataagtaatctggagatgacttaaagcatacagaaggatgtgcctaggttagatgcatgtatcgtaccatttcatatcagggacttgagtacccacggattttggtatctgcagatcctggaaccccttccctatggataccaaggaacaacagcactgggtctccttttggggtgatgcagatgttttgaagctaggcagaggtagtggttgcacaacattgtaaatgtactaaatgccaccaaattattcatttttaaatggttaatgtgttatgtgaatttcaccttaacaactaataatattataggtaaggcacaagttacatctgtagcacaaaaatggccctaatttttaaaacactgctccagcatagcaggtatcacatgtgaggtagcaaaagctggagatcaaagtgtgatacctggagacttatcagtaagggtcaaatgttttttcaggttttgagaatcattcttggaattgttccagaagatatatcgtataactcttcttagatgctaagataagaaggcagatatacactagctcattttgtgttattttctagagctttactccagtcaatttcttgggggcagcatttgtggaatcagtggttcatctgaagggctgtgctgtggaattactatgcatttgttttgtcttccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgatgcccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttgtattaatatgtagcaaaggcttttccaatgggtgaataaaaacacattccattaagtcaagctgggagcagtggcatatacctatagtcccagctgcacaggaggctgagacaggaggattgcttgaagccaggaattggagatcagcctgggcaacacagcaagatcctatctcttaaaaaaagaaaaaaaaacctattaataataaaacagtataaacaaaagctaaataggtaaaatattttttctgaaataaaattattttttgagtctgatggaaatgtttaagtgcagtaggccagtgccagtgagaaaataaataacatcatacatgtttgtatgtgtttgcatcttgcttctactgaaagtttcagtgcaccccacttacttagaactcggtgacatgatgtactcctttatctgggacacagcacaaaagaggtatgcagtggggctgctctgacatgaaagtggaagttaaggaatctgggctcttatggggtccttgtgggccagcccttcaggcctattttactttcattttacatatagctctaattggtttgattatctcgttcccaaggcagtgggagatccccatttaaggaaagaaaaggggcctggcacagtggctcatgcctgtaatcccagcactttgggaggctgaggcaagtgtatcacctgaggtcaggagttcaagaccagcctggccaacatggcaaaatcccgtctctactaaaaatattaaaaaattggctgggcgtggtggttcgtgcctataatttcagctactcaggaggctgaggcaggagaatcgctgtaacctggggggtggaggttgcagtgagacgagatcatgccacttcactccagcctggccaacagagccatactccgtctcaaataaataaataaataaataaagggacttcaaacacatgaacagcagccaggggaagaatcaaaatcatattctgtcaagcaaactggaaaagtaccactgtgtgtaccaatagcctccccaccacagaccctgggagcatcgcctcatttatggtgtggtccagtcatccatgtgaaggatgagtttccaggaaaaggttattaaatattcactgtaacatactggaggaggtgaggaattgcataatacaatcttagaaaacttttttttcccctttctattttttgagacaggatctcactttggcactcaggctggaggacagtggtacaatcaaagctcatggcagcctcgacctccctgggcttgggcaatcctcccacaggtgtgcacctccatagctggctaatttgtgtattttttgtagagatggggtttcaccatgttgcccaggctggtctctaacacttaggctcaagtgatccacctgcctcgtcctcccaagatgctgggattacaggtgtgtgccacaggtgttcatcagaaagctttttctattatttttaccttcttgagtgggtagaacctcagccacatagaaaataaaatgttctggcatgacttatttagctctctggaattacaaagaaggaatgaggtgtgtaaaagagaacctgggtttttgaatcacaaatttagaatttaatcgaaactctgcctcttacttgtttgtagacactgacagtggcctcatgttttttttttttttaatctataaaatggagatatctaacatgttgagcctgggcccacaggcaaagcacaatcctgatgtgagaagtactcagttcatgacaactgttgttctcacatgcatagcataatttcatattcacattggaggacttctcccaaaatatggatgacgttccctactcaaccttgaacttaatcaaaatactcagtttacttaacttcgtattagattctgattccctggaaccatttatcgtgtgccttaccatgcttatattttacttgatcttttgcataccttctaaaactattttagccaatttaaaatttgacagtttgcattaaattataggtttacaatatgctttatccagctatacctgccccaaattctgacagatgcttttgccacctctaaaggaagacccatgttcatagtgatggagtttgtgtggactaaccatgcaaggttgccaaggaaaaatcgctttacgcttccaaggtacacactaagatgaaagtaattttagtccgtgtccagttggattcttggcacatagttatcttctgctagaacaaactaaaacagctacatgccagcaagggagaaaggggaaggaggggcaaagttttgaaatttcatgtaaatttatgctgttcaaaacgacgagttcatgactttgtgtatagagtaagaaatgccttttcttttttgagacagagtcttgctctgtcacccaggctggagtgcagtggcacgatctgggctcactacaacctccgcctcctgggttcaagcaattctctgcctcagcctcccgagtagctgggattacaggtgcctgccaccacacccggctaatttttgtatttttagtagagacggggtttcaccatcatggccaggctggtcttgaactcctgacctagtaatccacctgcctccgcctcccaaagtgctgggattacaggcgtgagccactgcacccagccagaaatgccttctaatctttggtttatcttaattagccaggacacttggagtgcatcccgaagtacctgatcagtggcccctttggaatgtgtaaaactcagctcacttatatccctgcatccgctacagagacagaatccaagctcatatgttccatcttctctggctgtatagtttaaggaatggaaggcaccagaacagatttattgaaatgtttattagctgaagatttatttagacagttgaggaaaacatcagcacccagcagtaaaattggctctcaaagattttcttctcctgtggaaagtcagacctctgaggccccatccaggtagaagtactagtgcaagaagggcctctgctgtccacttgtgtttctgtgatctgtgggaacattgttaacgccacatcttgacctcaaattgtttagctcctggccagacacggtggctcacacctgtaatcccagcactttgagaggctgaggcaggtggatcacctgaggttaggagttcgaggccagcctggtcaacatggtaaaaccccgcctctactaaaaatacaaaaattagctggccgtagtggcgcacgcctgttatcccagctactcgggaggctgaggcaggagaattgcttgaacctgggtggtggaggttgcagtgagccgagattacaccactgcactccagcctgggtgacaagagggaaactccattaaaaaaatgtaattcccgtgtctgccatcttaagtgtaaaggtggctaaattatatagaaaaataagacaatatcatttcccaattacattcctttcctaccgcactctatgatgctagctgagatttttccaaaagaaaatggcttaaataaaaccctaagagaaagaaaaactttaaatccctccaaagctcaaaagtaatagaaacagatgagtttggagtcaggatttctctgtaagattgcctaggctgtgtactgcacatctccaggtgccactgttgacagagattataactacaatgtgaagtgaatggtgccactgacagttatgcaaaccgtccagagcatagccacctgatcctgctgggattcctcttgccagtccatcagcagttccccttgaaagtttcaccaaacatcccttaaatctgccctctcctgcccgtccccagtggaggtcctcatcatttttcacctgcatttttgcaggagctttcttatatccaccttcctccttttctctcagcccatcatctagctacacagtctccagggtaagctttcagaaaggcaatctcttgtctgtaaaacctaagcaggaccaaggccaagtttcttagcctgaaaaatgtgcttttctgactgaactgttcaggcactgactctacatataattatgcttttctaccccctcacactcaacactttgactccagcaatcccaaatccccagatccctaagtgtgctgtgctattttcacgtggctctcagacttggccagtgctgtttccattttggtctttattccccacatctctgcctggggggtagattctaccctgaaaaatgttcttggcacagccttgcaaactcctcctccactcagcctctgcctggatgcccttgattgttccatgtcctcagcataccatgtttgtctttcccagcactgacctaccatgtgtcacccctgcttggctgtaccttccatgaggctaggactatgtgtctcctttgttgactgctgttgccctagcatcttgcacagttccttgcacacaattagagctctataaatgtcaaataaatgtgttataattatatgtttaagatagttgttcaaataaactctaaataaccccaactccaagagtgttagcaagaaatataaattttacagaagaatggttggaggtggggagggtgtccacggagtgagttacctcacacaggcacggaaaaacttgaacctcctaaggacatttttaagctctctttcccattttctctcctggattcccattgcctggtctcatttctctcttctccaccacaccacttcctcaaaaattcctttagggtttgttcttaagcttagataggtttcccattctgaaatacaaaggcctgataattagccaacttaccttgttggggatgtggaaggcaagactctcagactccatgactcaggtatattgcaacaattaggctgaaagttccttgagagtaagtgtccaaatcttttcatgtttggttcccagggctcactacagttgttggtatatcataggcactctaatatcttcttaaagaatcaatatcattaaaatggccataactgcccatagcaatttacagattcaatgctatttctatcaaactatcaaggtcatttttgttttattttttttctttgagatagaatctcgctattgtcacccaggctggagtgcagtggcgcgatctcgactcactgcaacctccgcctcccgggttcaagtaattctcctgcctcagcctcccgagtagctgggattacacgtgcctgccaccacacctggctaatttttgtatttttagtagagacaaggcttcaacatgttggccaggctggtcttgaactcctgacctcaggtgatccacctgccttggcctcccaaagtgcagggattacagcatgagccactgtgcccggcccatggtaatttttcacagaatcagaagaaactattctaaaattcatatagcggccaggcgaggtggctcacgcctgtaatcccagcactttgggagacagaggcaggaggatcatctgaggtcaggagttcgagaccagcctgtccaacatggtgaaaccctgtctctactaaaaatacaaaaatttgccagtcgtgatggcgggcacctgtagtcccagctactcgagaggctgaggcaggagaattgcttgaacccgggaggtggaggttgcagtgagccgagatcacgccactgcactccagcctgggcaacagagtgagactccatctcaaaaaaataaataaaataaaataaaataaaattcatatagaaccaaaaaagagcccaaatagccaaagtaatcctgagcaaaaagaacaaagctggaagcatcacattacccaacttcaaactctactacaaggctatagcaactaaaacagcatggcactgctacaaaaacagacaggtagactaacggaacagaatagacaactcagaaataaagccacacacctacagccatctgaacttggacaaactcaacaatattaagtaatggggaaaggactccctattcaaaaagtagtgctgggataactggctatccatatacagaagaatgaaactagactgctacctatccccatatacaaaaattaaatcaagatggattaaagacttaaatgtaagatctcaaactaaaaaatcctagaagagccaggcgcggtggctcatgcctgtaatcccagcactctgggaggctgaggcggatggatcacctgaggataggagttcgaggccaggctggccaacatggtgaaaccctgtctctactaaaaatacaaaaattagctgggcatggtagtgtgtgcctgtaatctcagctactcgggaggctgagacaggagaatcgcttgagcctgggaggcagagtgagcccagatcgcaccattacactccagcctgggtgacaggagcaagattccatctcaaaaaaagaaaaagaaaaaaaaaatcctagaagaaaacctagtaaatgcccttcttatatcagccttgacaaagaagttatgactaaatcctagaaagcaattgcaacaaaaacaaaaatttacaagtgggatctaattaaactaaagagattctgcacagcaagagaagctatcaagggagtaaacagacagcctacagaatgggagaaaatattcacaaattatgcatctgacaaaggtctaatatccagaatctataaggaacttaaatcaacaagcaaaaaccaaataaccccattaaaaagtaggcaaaggacacgaacagacatgtctcaaaagaagaaatacaagtgaccaacgaacatgaaaaaatcctcatcatcactaatcatgagagaaatgcaaatcaaaagcacagtgagatatcatttcataccagcaagaatgactattaaaaaagtcaaaaaataacagatgttgcaagactgcagagaaaagagaacgtttatacactgttggtaggaatgtaaatacattcaaccactgtggagaacagtttggagatttctcaaagaactgaattgaactaccagtcgacccagcaatgccattattgagtatatgcccaaaggaaaataaattgttctatcaaaaagacaaatacacccatgtgttcatcacagcactattcacaatggcaaagacatgaaaccaaaccaggtgctcatcaatggtggattagattgtgtacatatataccaccatatggtacatatacactgtggaatactatgctgccataaaaaagaatgtaatcatgtattttgcagcaatatggatgtagctagaggccattattctaaacaaactaacacagaaacagaaaccaaataatgcatgttctgacttaaaagtgggagctaaacactgaatacacatgggcataaagatgggaacaatagacagtgggggctattagagaggcaagggctgaaaaactacctattcggtgccctgctcactatctgggtgacagagtcattagcactccaaagctcagcatcacacagtatacctttgtaacaaacctgcacatgtaccccctgattctaaaataaaagtcgaaggaaaacaacaaaaacaaaaagaaataactcctgagttggggtctccatctcttagttcagcctattggcagtcccctttttcaagttctaaggagcctgtactagactactcttcatttagtcccataataatccctctttcaattattttgccttcaaacctatagggaagggattggaaatgaagtttcagtcattccctaagtaaaatgtatatacatattttaattgaaacaggatttcactctgttgcccaggctggagtgcagtggtgtggtcatggctcactgcagcctcaacctcctgggctcaagcaatgcttccatctcatcctcccaagtagctgggactacaggctcgtaaattttttagagaacaaaaacacagtctttagatttaaacatgtgaaagcagaaattttaaaaatacaatgaaagagttggaagacagagttgaaattgttcagaaattacagtaaaaatactaagagataggaaatagtcaacttccaaatgagaagaatcacgaaagagagaacagaaaagatagaaaaaaaattatcaaagaaataattcaagaacatttccttaaagtgaagggcatgagattccaggtatattccacatatagaaaaatatcccatacaaaatcacattgttatgaattttcataacatgagggacaaaaaaagataatataagtaaccagagagggaaaaaataaataaacaaaacaagacaaataggtcatatacaaagtaatattcatcacaatagcttcatagttctcaataataacaaaaagcctttaaaattctggttgaagcagttcagacaatgccatcacccaaaaatatgccattttggcatactgattattattagctgaaagcacttgagaaacagcagactgtacaggaagggctttccaacctcctcttttctacctaaaaacaggctagaaaatttcccatgataaaggtgccctccctctactagaaagagaaaaacatccttatcaccagagatagggaatcaatgccaaaatggatctgaacaaacttattggaataacccttgtcttccactacttatccccaatatagctcttagtaatttccccaagcccctttgtcttgtcatttcttcacaaatttatcatttctttgtctaaaacatatataaacttgtctgctatggtgacttcttcgggtctacatttgcttgtgaggactcccaggtacatgtaaaattgtaataagacttgcgtgcttttctactgttaatctttcctgtgtcagtttaattcttaggcctagctggaaacttaagagggtagaacagaaatttttcctttcctacatggtgaagggacattctgtaataaaactagcctcaacattaaaaaaatgtgatgtaataaaaaacaaaggaaaaagaaaacaaaacagaaaagcaattaataacactaggaaacacgaggcattgtacaggataggaaacgtcctgttatgttacacaatgcaacagtgggtattgttttcatcattattataatgaaaatgctaaatagtgatttgaccaacaatccagtttaaaacatttggaggaatgtgaatgtttatggccagaaaatggggagaaaaatggttaaggaaacaaaatctcatcatctagagtgggaaggagactgataattcctaatatgaaccaaaaactcaaacttttttttttttttttgagatggggtctcgctctgtcgcccaggctggagtacagtggcacgatctcagctcactgcaacctctgcctcccaggttcaagagattctcctgcctcagcctcttcagtatttgggactacagttgcacactatgatgtctggctaatttttgtatttttagtagagatggggtttcgccatgttggccaggctggtctcgaactcctgacctcagatgatcagtccgccttggccccccaaagtgctgggattacagacatgagccattgcacctggcctgaaaactcattttatttagatatgttaagggaaatctcaaaataatcagctagaaaaattgaaaatggttgcccatgaggaggggagaactgttattatttatgtcaaataaaatttgtaggaagccattgatttggactgtgctcctgcactaggccccaatagaccaaaccacatggagtcactcttgctaaagttccacgtcaccaaaccaaagctaagtagtttatcttaccttctgggaaattaggggagagaaataatagacaaatccccaaacaggccagttttagctggcatataaggaagtcctctctgttttaaccgtattaggagagtaactttgaaaagaccgtccactttttggtccctgtttctgttttcttctgccttttctgcctataaagctaacttcctctgcccagctcactggagtaccttctctgaatttttagaagacaggctgccctgatccatgaattgcaaatgaaagccaattagatcatttaactaaattcattgtaattttgtcttttgacatttgtaaacaagccttgtagtacttgctaaacaatgggctgggcgcagtagctcacacctgtaatcctagcactttgggaggctgaggtgggtggatcacctgaggtcaggagttcgagaccagcctggtcaacatggtgaaactccgtctctactaaaaattcaaaagttagatgggcatggtagcatgtgcctgtagtcccagctactcaggaagctgaggcaggagaattgcttgaatctgggaggcagaggttgcagtgagctgagatagtgccactgtactccagcctgggcagcagagcaacactctgtctcaaaaaaaaaaacaaaaacaaaaacaaaaaaacaacttgctaaacaacatatgtttattatttggtaaattataaacaataaattcaaaactttaaaaagaaaacattttattgatagctcactgaatacaaatttataaaatattatttatgcattaagtttcagttacacattttcacccatcattacagatgtcatatggagttgctagagtatgagaagagcttcttcatcccaacagctttcaaagtgaagaggcgactcatgcctgtaatcccagcactttgggaggctgaggcgggtggttcacttgaggtcaggagtttgagaccagcctggccaacatggtgaaacctcgtctctactaaaaatacaaaaattagctgggcgtggtggcgcacacctgtaatcccagctactcaggaggctgaggcaggagaatcacttgagcccgtgaggtggaggttgaagtgagccaagatcatgccactgcactccagcctgggtaacaaagcaagattctgtctcaaaaaaaaaaaaaaaaaaaaagtgaacatctgggtcccccagatctcttcagagatatgtaatgttctcctttttccaactacataactctttaagctgggttttcttcatatactccaatgaaaacaacatattgcaacagatggaatgaagaggcaagtagaagaatccagctgttttctattaagccaaacattacaattgtcagctgaagaattctgagattcataaatttggaaagaaaagcttcatttctcataaaagattgcagcctgcagggtggccattctgacaggctaagaaatgtagtctctggccagaagccaaaaacagacactgagggtcagaagaataagatgggcatttatgctgaataggatggccaaatatacatattcaataaactacagtcatgaatattcatgaaaggagaaacatgcacatgctcaattgagcttcatgcctctccatgggacgcgtgtgcaaaaaatggcagcattagcatgatcagagggtggagttttctgtcctctgatatcaaaaggtgaaacagaggacacagaaaccctcactgcacatcctctgtaaactggccagaaccactccattgtgggcagtctgttatcaggaaggaatgctggttagttgtgcagaaactgcaaaaggaaggggcagtgtcagaccattggttgatatcagcggtgcagctcgtctttccaaagggctggtttctgtttaacctgtaggaaggaaatcctaatggcgtttagcaatggagagggtataacaacacatcatggcaagaactcagttttcaaggtttctctggggtccccttggccaagaggtggtgcatccgtttagtcagctgggggacttaggatttcatttttatttctcagagttttataaaactctaaaataattatttgacagccaggtgggagggggtccctggagaaactccaaccagcctgcctactagggtggagccttgggagtttgcagcagggaggagcctggcgcctcctcttcctatgtgaacctgggattctagcagcctggtgggaagcactgtagcaggagactctggccttgcagaggatccctgttcccctcatccctttatttccccttttcacttaataaaaccctgctttactcaccctttaaaccatctgcaagcctaaatttttgtggctgtgggatagacaagaaccttctctttagctgaactaaggaaaagtcctgcaatgatcccattcttcacaccaaatatgttttgtttcaaaagtatagttatttatcataaatatgtcattaatattgttaaatcaaatttagcctaaagctgcctccttatatagtttaagcttgacctaaaggtttctctgtacttagtgaattgtagcctacccagatgtgtaaacaagactgtgaactactcttgtgacaaacattggattttggccaatcaaaggaggtcaactcttgacactgctttcaaataaggcaaatattgagctgtaaacaatctggctgtttctatacctcacttctgttttctgtacgccacttttctgtctctgtccataaatgttcttccaccacgtggctgtgctggagtctctgaacctactctggctgaggaggctgcccaattctcaaactgttcaattaaactcggttaaatttaatttgtctaaggttttcttttaaccatataaacaagtgagtttatgattgttatgtcttttttcttttcttttttgagacaaggtcccactctgtcccccaggctggaatacagtggcatgatcacggctcactgtagtctcgcactcccaggctcaagcgatcctccatctcagcctcctgagtagttgggagtacaagtgcatgcaaccatgcctggctaattttttttttttttgtattttttgtagagatagggttttgctacattgcccaggttgatctcgaactcctgagctcaagtgatcctcttgcctcagccttccaaagtgctgggaccacaggcatgagtcaccacacccagctattatttctaaattaatgaacagatgaacattttcaaaatttctcagttttaattttaaatatgattaaaaggatagatataacacacaaacaaaagctctatggagtcctctataactcaagaatataaagggtcctgagatttttctttaaagagaaccactgcactctcctggcctactagctctccgcaatccatcctgcttctccccttggcaggagagacctgttctagaccctcaaggacccctcataacatcacctagctattatctaaggaatctttctccatttggacttcccatttttttcttccccctttaaggtccccttattcttttcatctaattttgtgtgccacctgcagagtccttcttcttcttcttctccttctccttctccttcttcttctcagagtcttgttctgttgcccaggctggattgcagtggcacgatctcggctcacttcagcctctgccttctgggttccagtgattctcctgcctcaggctcctgggtagctgggactacaggtacccaccatcatgactggctaatttttttgtatttttagtagagacggggtttcacaatgttagccaggatggtctctatctcctgacctcgtgatccggccgcctcggccttccaaagtgctgggattacaggcatgagccaccgcacccggcgactaatttttttttttttttttttttttgagacggagtctcactctgtcgcccaggccggactgcggactgcagtggcacaatctcggctcactgcaagctccgcttcccgggttcacgccattctcctgcctcagcctcccgagtagctgggactacaggcacccgccaccgcgcctggctaattttttgtatttttagtagagacggggtttcaccttgttagccaggatggtctcgatctcctgacctcatgatccacccgcctcggcctcccaaagtgctgggattacaggcgtgagccaccgcgcccggccggcgactaatttttatatttttagtagagacggggtttcgccatgttggctgggctggtcttgaactcctgacctcaggtgatccgcccgccttggcctcccaaagtgttgggattacaggcatgagccaacgcacccggcctgagtcctgcttcttccagatctggtgcccagtcctgacgccagaaagggggtcttgttccagaccccaagagtgttcttggatcttgcctgggaaagaattcagggtaagtcgcagagtataatgaagttaagatagttaattagaggctactcaattacagagtagggcatcctcagaaaacaagaggaggaaggcgctaccttaaatgtagtgcttgcttatgtaggttgtataagaattgtgtactttattacaaaggcttgtgatcagcttgtgacaggctattggtactgttattttcctgttactattgatttcagcaagaatttatgagtacactattatatttaaggcaaaacctattccttaagaatgctttttgttcttaaaatactgggacatttccataagttctgagtctttagttagcaacattaactcattccctcaatcataaacatctcatgaccaagagtgcccagttcctggggaatgtaacccagcaggtttggctttattcggcctttattcaagatggagtcactctggttaggacacctctgacagtccctggaaatccaaaggaacccttctgtgtggcacagggaatggaagaaagaaagagatgaggcaggaaaatagggtctggaggcagaaaacataagccgattcacacttcagctatgacaggaaatatcctctccatagggcgtatgcctgtaactttacttcatcctcttcatttacataggacgtatcctaagtaaccaatggaatcgtctagagggtatttaaactcccaaaaattctgtaacagggcctttgagcccctatgctcgggcccgctcccacactgtggagtgtactttcattttcaataaatcccttcattccttccttgctttctttgtgctttgtgcattttatctaattctttgttcaagacgccaggaacctggacgccctcccctggtaatagagagatgagcctttcaaatgacctgactcctttatcccagccaggtgtgtgcccgaccctgaaaggaggaatagggagggggacgttcaacccggcctcccgctctgtgttagcagcgtctggatgggtcagggtggaggtgggggtgttctaccctgctatttgctcctagagaagcttctctgcttcactagtctcacagttctaaaggcaagaacagccctagtgggatcttccaaggattttagaaaagaatgaataagggaaaaattaaaatattgcagggtgccataaaaacatcccagtaaaacaaacacctttctagatgctcattggaacgtaaatggagctcagcccccatcccttcacaccagatccagtcttcatctttgtggttcactgccccctcaccactcaggaggaaaaccccagcttctgttctggctccccttctctcacttagaatttttcaccagagtttcagaaagatttgtcaggaccactccatgcccaaggtaaaaagtgtaagtggtacaaaaaggtagaaactcatcagacccccaaagagtgtcatttaaccatacaaagccctgataaactccagggcagaagaaaaagctgcatccttgactccactggggcattcttatgtaaactaagatccaagaactgcatcaggagagaaatcaagagccctggggatgttaggatgagccctagaggtgctaagacaggttatttgaaaaaccaaaaagtagactgagattcccttccttttcagggaagaattgagacctttcctttcttactgttcagagtgggggctgataagggtaattatttcctggagccactggctactgccctgggaaggaaatccgctgggttgggggagggaggaaggcagaaccaggcattaactctccctccactacatccctttcccgtacccctcccctcctctccttccccccactccctgcccccgccctccgaaaatgacacttggcctgagaaaggaggaaggtagaataggtggacacttcccttgtcctgctccaggggtgtctcagtgacaaggagatgtgaaaaaagaaggaatcccaaggctccccttggaaagaagggagatctccaggggctttgggaagtcaggttagtactgggaaggctgaagactcccagtagatagcgttcagggctgcatttggctgcaatcctataaaatacattcttctctaaggttggatacaagcatttagaagactggccattaaaaaaataaacagtattaataatattaataatcatgagtgtcagtagtgttgaattttttctggaatcctttcccaagttgcctaatgcccagagaaggaaaataacagtgtttagtagacataaattataggattagtgcaagtagctattgagatgatgagccaaggcttgtaaattggttttgttttggttttcctaattagatgtttgcgcctatctgtgtatgtgtgtgtgtgtttgtgcgtgtgcatgctcgcatgtggttaatttcatgacttttgcctctggctcttcctgattaaaaaaaatacttaaaatggtaggaagtggcacacacccttgatggacctgtgtttatattaaagaattggcttagtaaatttaactgggacaaggaaactgtgaaggactgtatttttgccattatttaataattcatatattcaaccgttactgattgcctattttgaaccaggccacgtgctaggatacaatggttaacaaacacattccctcccctcaaggaattcatggtctagtgaaatacagagatagaaaagaaatagaaaagtatatcaataaaatgcattgtggaaagagttatggtcatagtgtgtactatatgcttatagaggctgcctttgtataaacatacataagactgctttttaaattataaaaggcagtacataggccaggcgtggtggctcacacctgtaatcccagcactttgggaggccgaggcgggtggatcatctgaggccacgagttcgagaccagcctggccaacatggtgaaaccccatttctactaaaaatacaaaaaaaaaaaaaaaattagccaggtgtggtggtgggcgcctcatcccagctatcaggaggctgaggcgggagaatcacttaaacccagacggaggttacagtgagctgaggtggagccattgcactccagcctaggcaacaagagcaaaactccatctcaaaaaaaaaaaaaaaaaaaaaaaggcagtacatagtacaaactgcttgggttttgttgttgttgttttactgtaccatataggttggagatcattccacctagtagctgaacattttaagcagatcatctggctacaggcagtgagtaggatgaactgggagagtgatgagtgagttagagagttagggagggagggtgctgtcggagtgttaccggaaaggggtcccgatccacaccctaagagagggttcttggatctcgcacaagaaagaattcagggcgagtccatacagtaaagtgaaagcaagtttattaagaaagtagagaaataaaagaatggctactccatagacagagcagccccgagggctgctgttgcccatttttatggttattccttgatgatatgctaaacaaggggtggattattcatgcctccctttttagaccatatagggtaacttcctgacgttgccatggcatttgtaaactgtcatggcgctggtgggggcgtagtagtgaggatgaccagaggtcactctcgtggccatcttagtgttggtaggttttggccggctccaacaccggcttgttgttttatcagcaaggtctttatgacccatattctatgcccacctcctgtctcatcctgtgacttagaatgccttaactgtctgggaatgcagcccagtaggtttcagccttattttacccagctcctatttaagataaagttgctctggttcacacgcctctgacaagaacatcttcatgcctgtgcctggttgagagagggaggcctctgcgctgctgctggatctagtgaagattcactcagtctctcaaattcctctacagtttctctaatggaagagaaaagtggtgttattgctgctagggagcaacctagaagttattttatttatgccatagatatggtgggctaagcactgtgccaacgttcaataagtcactgcagattctccataaattattgtgacaagtacaattgtttgtaaggcttagatctaggtgtgtaagtccaaagaagggtgtgaagcatctgtatttctgttatgtagttattaggaaaaaggatgttggggccttaaaatggccatttttaacatttccaaacttgtgttgaattctaagattttataattgtatgtttccagttgagaagagctttgatattggtagctctaaataaataaataccgttgacctggaagagaaggtaaagtttagggagaggcctttttttagctttatatttaaacattttttataaatgtgattcatgggccaggcctggtggctcacacctgtaatcccagcacttttggaggccaatgcaggtggatcacttgaggctaggagttcgagagcagcctggccaacatggtaaaaccccatctctactaaaaattagccaggtgtggtagcacacacctgtaatcccagctactcaggaggctgaggcaggggaatcacttgaacccaggaggcgaaggttgcagtgagccgagattgtgccactgcactccagcctgggtgacagagtcagactccgtctcaaaagcaaaacaaaacaaaatgttattcataatgctcgggttgtaactatagtacttatctagcaaaagcttgcttttttttttttggctttgactaattgaaactgcaagagcttactggcagagtggtgtactggtcaatatttaaccaattctccaaaggggaaaaaccctgatttgtatgtaggatttgtcagtttccatggtataaatagtcttcccacagctggtagggtgaccaacttgttctggtttgccaggggctttcccatttttaggcctgaaagtcctgaatcccagaaaattcctcattccccaggaaatagcttgattggtcaccctaatggctggttgcaagctcccgatatgacagaactggacgagaagttgggcagagatgtgcacatggtaccagcctatgccaggagcagcggcctccagcaccccactgtcagggagtccttggcccagtagaggatggttagcagggcccggctgttgttcatattagctctcaaatttaccaccaaccctgtattagtttcctggagctgctgtaacaaagttccacaaacgggggtcttaaacacagaaatctattatctcacagttctggagggcagaaatagaaaattaaggtatgagcaggactctgctcttttgatggctctagataatccgttgtatgtcttttcctcagcttctggtttcacaggtaatctttggcgatccttgacttgcatctgtgtaactccagtctctacctccatcatcctgtggcattcttctttatttttctttctttttttcttttcgagacagagtttcgctctgttacccaggctggagtgcagtggcgtgatctcggctcactgcaacctctgcctcccaggttcaagcgattctcttgcctctggctcccgagtagctgagattacaggtgtgcgccaccacacccagctaatttttgcatttttagtagaggcggggtttcaccatgctggccaggctggtctcgggctcccgacctcaggtcatctccctgccttggccttctaaagtgctgggattacaagcgtgagccactgcactcggcccatggcattcttcttttggtgcctttgtcttcactgacttcttgtaaggaaatcagtcgtattggattagaggcctaccttattccagtatgatctcattgtcttaatttaactaaaacatctgcaacaaccttatttctaaatgaggtcacattctgaggtattagggtttagtacttcaacatatcttttttttttttttgagacagggtctcattctgtcactcaggctggagtgcagtggtgcaatcacacagctcactgtaactttgaactcctgggctcgagcagtcctcctatctcagcctcccagataggtaagattacaggtacatatcaccatgcctagctaatttttcaaattttttataggggctgggcccagtggctcacaccttgtaatccctgtaatcccaacactttggtaggctgaggcgggcggatcacttgaggtcaagagtttgagaccagcctggccaacatggtaaaatcccatctctactaaaaaaaatacaaaaattagccggatgtggtggtgggtacctatcataccagctactcacaaggctgaggccggaaaatccctggaacccgaggggcggagatcgcagtgaaccgagatcacgccatgcactccagcctgggtgacagagcaagacataaccttaaaaaagaaaaaaaaaaatgtagagatgaagtcttgctgtgttgcccaggctagtctcaaatgcctgggctcaagcaatccttctgcctcagtatcccaaagtgctaggattacaggcatgaggcactgcaccaggcctacatcctcttttttttttttttttttttttttttttgagatagagtcttgctctgtctcccaggctggagtgcagtggcacgacctcggctcactgcaacttccacctcctgggttcaagtgattcttctgcctcagcctccagagtagctaagactacaggcataatatctctcttagatatgacaaataatatcacagagtgtacacccactgtgatgttaggagtaatacctccctatgatattacaagtaatactgcctttagatactacaaataatatcacagggtgtacatctactgtgatattaggagtaatacctcccttagatattacaaataatatcacagggtatacacccacggtgatattaggagtaatatctctcttaagcgatcctcccatctcagcctcacagaattaaaggaattacaggaagagctgctatacctggctggatctatgttttaaaaatataacccagataaccctgtggtcagtgtctaagatgaattggattagaccaagggagaaaaactaaagatgggaatactagtttgggactttgcttgcttgcttgctctcatttagaaaacatttagtagttctacaatgctcaggcactgttctgggagtcacaaatataggattgaataaagtaaataaagcacttgctctcctggagctcacttttcactgggggaatgcagatagtagacacatacatctatagtatcagtaagtgctaatagaaaaatgaagcaggtgagatggatcatgctgagtagaatgtatcttcttttccttccttccttccttccctccctccttccttctttccttccttctttctttccttcctttcttcttttctctttcttcctttctctctctttctttgctttttattgtcttaaaatgtacataacataaaatttaccctcttaaccatttttaagaatacaattcaaggccgggcatggtggctcacacctataatcccagcattttgggaggctgaggcaggcggatcatgaggtcaggagtttgaggccagtctggccaatatgatgaaaccccatctctactaaaaaatacaaaaattagccaggcttggtggcacatgcctgtagtcccagctacccgggaggctgaggcaggagaatagctggaacctgggaggcagaggttgcagtgagctgagatcgcaccactgcactcctgcctggacaagagagcaagactctgtctcaaaaataaataaataaataaataataataataataataatacaattcagtagccttaagtacatttgcattgttatgcagccatcaccaccatccatctccagaatttttttgagtggagctctttttaatagagtagttgaaggcctctgtgacacagtagcatctgagcagaagcttgaatgaagtgagaaaagaatccttttgcatagtttaggggaagtatgttccattcctggtcctggaaatagttaagactatcacaatagtgcaggagaaagatgatacaatacagtttgtgtagctgaaaccccgtcttcagaatgtaaaggagaacagatgggaagtcatgttcctcccagaagtaattcatgtagcagagaagccaatgcagatccacgagacagacaattcagtgctctgcacaagaactgtgctttaagcatggagaggatttttgtatctgtcctgggatcctacatcaaacagcatgtggtgattgtgaacacaaacgtacaagactgtgaaccctaccaagtttccttcttccattagatatgaataaggagtcatgagtttcctttggaatgtcctttagcctgttggtacatgttttgcctgtgacgaatgcagttactcataaatcattgagcacattgggtacagagggcaaaagataaattcctgtatttcctctattcggtcaacagaaatacctctaggccataatccattcatccaatctaataattttgccatccataaaaccttcaggtgttctgaattcaacatctttttttttttttttttttttttttgagacagagtcttgctctgtcacccaggctggagtgcaatggcaggatctcggcttactgcaacctccgcctcccagattcaagcgattctcctgcctcagcctcccgagtagctgggattacaggtgcccgccaccacgcccagctaattttttgtattttcagtagagacggggtgtcaccatgttggccaggctggtctcgaactcttgacctcaggcaatccacccgcctcagcctcccaaagtgctgggattacaggcgtgagccaccatgcctggctgaattcgacatcttgcacctaattcctgttcagttaaagacccaaatcatgatctctgacttacctggatatttgaaagattaacttgctgtggtgataccatactagagtcacaaaatcaagccctaccctgccacagccacctaaaggaaattaggtgatatacaaaagaaattgaccatattgttgtccttttagtgactctcctaattttcttcccctgaaaacttacagagaaatttgagtatgtttgccttaggtggatgcttgtttttttattgatatgaaaagcagtaagaggaaatggagttttttggcctgttaaggaagggcagccactgtaaacacagttgagtgcaaattcacagtgttagaatgttgaagtgtatataatgattttgcaaaattttctacaaggctgatacagtatccaatcaggactaggattagatatattgtcatgtatgtttgcgcaggaaatgcagagactctaaggtgctacaactgcaatttgacatgtgggatagttcactggtaactgttgatctccctgaggtttaagtttacagttccacagctctttatctgaaactcttgggctatgtgttatggaatttagaattttttccgaaatacgttgcatatattgtatattatgacatgatacctccaagaaagacttggagtcacatcctataaacaaacacatgaatatatcccagtgaaatgtatgactatttttactaaaacaaatgagaatcataaatagacttacattacttcaggtcagattttgctgccgaattagtttgggcatcgaacttttggtttcagagacaaaactgtgaaattttagattatattatggggttgtggacccatgtaaccctcctctccgtaattcctaaaagcaagcaattgcatcaaccagtctcatgagtagctgcgattctagaaatcaagaatccggatctgaaattagccgggcatggtggcaggcacttgtaatcccagctactggggaggctgaggcaggagaatcgcttgaacccaggaggaaactgcagtgagctgagatcgtgctgctgcactccagcctgggcaacagagtgagactctgtctcaaaaaaaaaaaaaaaaaaaaaaaaagaatccagatctgggcaggaccgaattgctgacatgcccccggtatagcagagacgttttgcctacatgttacacacctgagtaatagttgtcagcagctgatgaagaagatgaatgtgctcttaatgtccatctttgatttccagtcattttgcttctgggtcttggcttcctgaggaaagaagtctccagtaggtgaatgcagtgatatggagaatactttcttctggctgcatgcagtaactcacacctgtaatcccagcacattgggaggctgaggtgggcagtgcacttgaggttgggagttcgagaccagcctggccaacatggcaaaaccccgtctctactgaaaatacaaaaattagctgggcgtggtgacagacacctgtcatcccagctactcggtaggctgaggcatgagaatcacttgaacttgggaggtagaggttgcagtgagccgagatcgtgcctctgcactccagctgggcaacacagcgagactctgtctcaaaaaaaaaaaagtgtgtgagagagagtactttcttcctgtttcctcataggccagttctctctggcatgtgagtttaacatcagtcacctccttcacacacagcgggtgcattcgtaataggaggtccttagctgggagtttttatggcacatcagtggggcgtgaaaacaccacataggagctaatatatctttgctggctgctttctccggctccgcagcagacagaaaccctatgaatcatatccaggggtcaggtgcaggcaacagacaactaatatctcccaagtgagttgaaaaggatcttgttacccagcatcctaaggaggttgtagccttgggaaccacaggcaagaataattaactcagctcctcggttagtgcctcttcagttcgagatggaatttatttgcaggcatggctccttaatatgccaaacccatgctcaagacatactccttctcctggaaggttaacgtggctcctgtggctgttccatccctgaggaaaagtgaggaccatgctctccaaacaggccatgtgctggactacctctgtttctgtctcctgggattccaatcagcaagtgagcaacgaagcaacccagacagtgtggttcataggatggctgggtaagtggctgtttgttttttccttactgtggatatgtatcagtgaaggaatctgtagaacattcttgatgggaacatttagtcatatcaagtcaataaattaatgtttaggctgggcgcagtggctcacgcctgtaatcccaacaccttgggaggccaaggcgggcagatcatctgaggtcaggagttcaagaccagcctggccaacatggtaaaatcccgtctctactaaaaatacaaaaattagctgggtgtggtggtgcatacttgtagtcccagctactctggaggctgaggcaagagaattgcctgaacctgggagatggaggttgcagtgagctaagagtgcaccattgcactctagcctgggcaacagagtgagactctgtcaaaaaaaaattaaaaaaaaagaaaaatcattattttatttttgacttattattaatataaataattatatcttggccgggcatagtgtctcatgcctataatcccagcactttgggaggccagggcaggcagatcacttgagccaagaagtttaagaccagcctgggcaacacggtgaaaccctgtctctacaaaaaatataaaaaattagctgggagtggtcagcttgcctgcagccctagctacctgggaggctgaggtgggaggatcacctcggcccaggaggtagaggctgcagtgagccatgattgtaccactgcactccagcctgggtgatagagtgatgagaccctgtctcaaaaaaaaaaaaaaaaaaaaaaagaaagaaagaaagaaaaaagaaaggaaaagaaatcatatattggtgaggagacaattcaacacatattttttattgaacacatactatgtgtcagggtaccagatataagctctatctacaaggattttaggagctggagtatgtgtatggggggatgtatgagtgtgtataacaaagacgactcctggggaagaagaggaagacaagccccagaggtatactgcataggcataatacacaacaggctagcaaagaagcaaaccatgggtatggtagagagaatcagaggatacattggggaccatgtctagtgagtgaggtcaggagagacttcaataatctgagtgaatttagacatgggccttgaaaagtggacaaggtttgttgttgttgttgttgttgttgttgttgttgttgttgttgttgtttttgagatggagtctcattctgtcgcccaggctggagtgcagtggtgcgatctcggctcactgcaagctccgcctcccaggttcataacattctcctgcctcagcttcccgagtagctgggactacaggcgcccgccaccacgcccagctacttttttatatttttagtagagacggggtttcaccgtgttagtctggatggtctcgatctcctgaccttgtgatccacccaccttggcctcccaaagtgctgggattacaggcgtgaaccactgcggccggcctaaatttgttttaaaagtacgcataggaaggctgggggctgtggcttatgcctgtaatcacagcactttgggaggccaagacaggcagatcacgaggtcaggagatcgagaccatcctggctaacacagtgaaaccccgtctctccaaaaaaacaaaaaattatccaggcctagtggcacacgcctgtagtcccagctacttgggaggctgaggcaggagaatcgcttgaatctgggaggtggagggtgcagtgagccactgcactccagcctgggtgacagagcaaactaggtctcaaaaaaaaaaaaaaaaaaaagtacatgtgggggacaggtgcagtgtctcagcctgtaatcaatcccagcactttgggaggctgaggtgggtggatcacttgaggtcaggagttcaagaccagcctggccaacatggagaaaccccatctctactaaaaatacaaaaattcgctgggcgtggtggcgcacgtctgtagtcccagctactgggaagactaaagtgagagaactgcttgagcccagaggtcgaggctgtggtgagcggtgatttcaccacttcagtctagcctgggtgacagagagagaccctgtctcatataaacaaataaataaaag

SEQ ID NO: 5 - DNA, 27 bp, Artificial sequence
  Synthetic construct
gtcggaacaggagagcgcacgagggag

SEQ ID NO: 6 - DNA, 20 bp, Artificial sequence
  Synthetic construct
gtggaagcccaatacgtggc

SEQ ID NO: 7 - DNA, 20 bp, Artificial sequence
  Synthetic construct
gctttcctggaacgaggtga

SEQ ID NO: 8 - DNA, 20 bp, Artificial sequence
  Synthetic construct
ggatttcccagcatctctgg

SEQ ID NO: 9 - DNA, 20 bp, Artificial sequence
  Synthetic construct
tcccggttgcatttacactg

SEQ ID NO: 10 - DNA, 20 bp, Artificial sequence
  Synthetic construct
gggttgtcagcagagttgtg

SEQ ID NO: 11 - DNA, 20 bp, Artificial sequence
  Synthetic construct
agggggagcttagggtcaat

SEQ ID NO: 12 - DNA, 20 bp, Artificial sequence
  Synthetic construct
tggcatcttcaagaccctca

SEQ ID NO: 13 - DNA, 20 bp, Artificial sequence
  Synthetic construct
ggagaaaagggtggggaaga

SEQ ID NO: 14 - DNA, 23 bp, Artificial sequence
  Synthetic construct
aagccatacacgtttgaggacta

SEQ ID NO: 15 - DNA, 21 bp, Artificial sequence
  Synthetic construct
ttggcgtctgcttgttgatca

SEQ ID NO: 16 - DNA, 19 bp, Artificial sequence
  Synthetic construct
ggcggagcgggcggcagac

SEQ ID NO: 17 - DNA, 20 bp, Artificial sequence
  Synthetic construct
ggggcgtgcaggtcgcatcg

SEQ ID NO: 18 - DNA, 21 bp, Artificial sequence
  Synthetic construct
ccaacgtggcctcaaccagat

SEQ ID NO: 19 - DNA, 21 bp, Artificial sequence
  Synthetic construct
gggtggcccaaagttccagat

SEQ ID NO: 20 - DNA, 26 bp, Artificial sequence
  Synthetic construct
catttgaacctccactacctccagat

SEQ ID NO: 21 - DNA, 23 bp, Artificial sequence
  Synthetic construct
tgtccaatgtccccaagttcctc

SEQ ID NO: 22 - DNA, 21 bp, Artificial sequence
  Synthetic construct
gttgcagtaagccaggaccac

SEQ ID NO: 23 - DNA, 23 bp, Artificial sequence
  Synthetic construct
gatccacccgcctcatttatttg

SEQ ID NO: 24 - DNA, 24 bp, Artificial sequence
  Synthetic construct
gaggccatatcccagaagaaaact

SEQ ID NO: 25 - DNA, 21 bp, Artificial sequence
  Synthetic construct
caggcagcatgaatggaggag

SEQ ID NO: 26 - DNA, 25 bp, Artificial sequence
  Synthetic construct
caggactgaaagacttgctcgagat

SEQ ID NO: 27 - DNA, 26 bp, Artificial sequence
  Synthetic construct
cagcaggtcagcaaagaacttatagc

SEQ ID NO: 28 - DNA, 22 bp, Artificial sequence
  Synthetic construct
ggctgcccagaacatcatccct

SEQ ID NO: 29 - DNA, 23 bp, Artificial sequence
  Synthetic construct
atgcctgcttcaccaccttcttg

SEQ ID NO: 30 - DNA, 20 bp, Artificial sequence
  Synthetic construct
agatgatcgccaagagcgag

SEQ ID NO: 31 - DNA, 20 bp, Artificial sequence
  Synthetic construct
atccccagcagctctttcac

SEQ ID NO: 32 - DNA, 20 bp, Artificial sequence
  Synthetic construct
tgcggaattcagtggttcgt

SEQ ID NO: 33 - DNA, 20 bp, Artificial sequence
  Synthetic construct
agctacaacaaggcaaggct

SEQ ID NO: 34 - DNA, 21 bp, Artificial sequence
  Synthetic construct
gattggttgccagtgcttaaa

SEQ ID NO: 35 - DNA, 20 bp, Artificial sequence
  Synthetic construct
tcaggtgatccaccttccta

SEQ ID NO: 36 - DNA, 14 bp, Artificial sequence
  Synthetic construct
tgcccataatctca

SEQ ID NO: 37 - DNA, 19 bp, Artificial sequence
  Synthetic construct
gttgcagtgagctgagact

SEQ ID NO: 38 - DNA, 12 bp, Artificial sequence
  Synthetic construct
agtgcagtggct

SEQ ID NO: 39 - DNA, 20 bp, Artificial sequence
  Synthetic construct
atgagccaccgcgtcctgcc

SEQ ID NO: 40 - DNA, 20 bp, Artificial sequence
  Synthetic construct
gatttcctggcaggacgcgg

SEQ ID NO: 41 - DNA, 20 bp, Artificial sequence
  Synthetic construct
aagtcctaacttttaagcac

SEQ ID NO: 42 - DNA, 20 bp, Artificial sequence
  Synthetic construct
tccggagttcaagactaacc

SEQ ID NO: 43 - DNA, 20 bp, Artificial sequence
  Synthetic construct
agtcttgaactccggacctc

SEQ ID NO: 44 - DNA, 20 bp, Artificial sequence
  Synthetic construct
ctaggaaggtggatcacctg

SEQ ID NO: 45 - DNA, 20 bp, Artificial sequence
  Synthetic construct
caggcgcgcgacaccacgcc

SEQ ID NO: 46 - DNA, 20 bp, Artificial sequence
  Synthetic construct
gagaatcgcttgagcccggg

SEQ ID NO: 47 - DNA, 20 bp, Artificial sequence
  Synthetic construct
ccgcagcctctggagtagct

SEQ ID NO: 48 - DNA, 20 bp, Artificial sequence
  Synthetic construct
cggagtgcattgggcgatct

SEQ ID NO: 49 - DNA, 20 bp, Artificial sequence
  Synthetic construct
aaagaaaagttagccgggcg

SEQ ID NO: 50 - DNA, 20 bp, Artificial sequence
  Synthetic construct
caagatcgcccaatgcactc

SEQ ID NO: 51 - DNA, 19 bp, Artificial sequence
  Synthetic construct
tttcaagccgtggcgtaac

SEQ ID NO: 52 - DNA, 19 bp, Artificial sequence
  Synthetic construct
gacgcccattttgcggacc

SEQ ID NO: 53 - DNA, 19 bp, Artificial sequence
  Synthetic construct
agttacgccacggcttgaa

SEQ ID NO: 54 - DNA, 19 bp, Artificial sequence
  Synthetic construct
ataccatgtcctccccttg

SEQ ID NO: 55 - DNA, 19 bp, Artificial sequence
  Synthetic construct
ataatcccagctactcggg

SEQ ID NO: 56 - DNA, 19 bp, Artificial sequence
  Synthetic construct
gtctcgaactcccaacctc

SEQ ID NO: 57 - DNA, 19 bp, Artificial sequence
  Synthetic construct
cactttgggagggcgaggt

SEQ ID NO: 58 - DNA, 19 bp, Artificial sequence
  Synthetic construct
tccagcctgggcaacaaga

SEQ ID NO: 59 - DNA, 20 bp, Artificial sequence
  Synthetic construct
taaaagttaggacttagaaa

SEQ ID NO: 60 - DNA, 20 bp, Artificial sequence
  Synthetic construct
actttgggaggcctaggaag

SEQ ID NO: 61 - DNA, 20 bp, Artificial sequence
  Synthetic construct
tttgtattttttagtagata

SEQ ID NO: 62 - DNA, 20 bp, Artificial sequence
  Synthetic construct
gccgcagcctctggagtagc

SEQ ID NO: 63 - DNA, 20 bp, Artificial sequence
  Synthetic construct
cccatgctgtccacacaggc

SEQ ID NO: 64 - DNA, 20 bp, Artificial sequence
  Synthetic construct
ttccctcttgttgcccaggc

SEQ ID NO: 65 - RNA, 20 nt, Artificial sequence
  Synthetic construct
augagccaccgcguccugcc

SEQ ID NO: 66 - RNA, 20 nt, Artificial sequence
  Synthetic construct
gauuuccuggcaggacgcgg

SEQ ID NO: 67 - RNA, 20 nt, Artificial sequence
  Synthetic construct
aaguccuaacuuuuaagcac

SEQ ID NO: 68 - RNA, 20 nt, Artificial sequence
  Synthetic construct
uccggaguucaagacuaacc

SEQ ID NO: 69 - RNA, 20 nt, Artificial sequence
  Synthetic construct
agucuugaacuccggaccuc

SEQ ID NO: 70 - RNA, 20 nt, Artificial sequence
  Synthetic construct
cuaggaagguggaucaccug

SEQ ID NO: 71 - RNA, 20 nt, Artificial sequence
  Synthetic construct
caggcgcgcgacaccacgcc

SEQ ID NO: 72 - RNA, 20 nt, Artificial sequence
  Synthetic construct
gagaaucgcuugagcccggg

SEQ ID NO: 73 - RNA, 20 nt, Artificial sequence
  Synthetic construct
ccgcagccucuggaguagcu

SEQ ID NO: 74 - RNA, 20 nt, Artificial sequence
  Synthetic construct
cggagugcauugggcgaucu

SEQ ID NO: 75 - RNA, 20 nt, Artificial sequence
  Synthetic construct
aaagaaaaguuagccgggcg

SEQ ID NO: 76 - RNA, 20 nt, Artificial sequence
  Synthetic construct
caagaucgcccaaugcacuc

SEQ ID NO: 77 - RNA, 19 nt, Artificial sequence
  Synthetic construct
uuucaagccguggcguaac

SEQ ID NO: 78 - RNA, 19 nt, Artificial sequence
  Synthetic construct
gacgcccauuuugcggacc

SEQ ID NO: 79 - RNA, 19 nt, Artificial sequence
  Synthetic construct
aguuacgccacggcuugaa

SEQ ID NO: 80 - RNA, 19 nt, Artificial sequence
  Synthetic construct
auaccauguccuccccuug

SEQ ID NO: 81 - RNA, 19 nt, Artificial sequence
  Synthetic construct
auaaucccagcuacucggg

SEQ ID NO: 82 - RNA, 19 nt, Artificial sequence
  Synthetic construct
gucucgaacucccaaccuc

SEQ ID NO: 83 - RNA, 19 nt, Artificial sequence
  Synthetic construct
cacuuugggagggcgaggu

SEQ ID NO: 84 - RNA, 19 nt, Artificial sequence
  Synthetic construct
uccagccugggcaacaaga

SEQ ID NO: 85 - RNA, 20 nt, Artificial sequence
  Synthetic construct
uaaaaguuaggacuuagaaa

SEQ ID NO: 86 - RNA, 20 nt, Artificial sequence
  Synthetic construct
acuuugggaggccuaggaag

SEQ ID NO: 87 - RNA, 20 nt, Artificial sequence
  Synthetic construct
uuuguauuuuuuaguagaua

SEQ ID NO: 88 - RNA, 20 nt, Artificial sequence
  Synthetic construct
gccgcagccucuggaguagc

SEQ ID NO: 89 - RNA, 20 nt, Artificial sequence
  Synthetic construct
cccaugcuguccacacaggc

SEQ ID NO: 90 - RNA, 20 nt, Artificial sequence
  Synthetic construct
uucccucuuguugcccaggc

SEQ ID NO: 91 - RNA, 76 nt, S. pyogenes
agagcuagaaauagcaaguuaaaauaaggcuaguccguuaucaacuugaaaaaguggcaccgagucggugcuuuuu

SEQ ID NO: 92 - RNA, 77 nt, S. aureus
uaguacucuggaaacagaaucuacuaaaacaaggcaaaaugccguguuuaucucgucaacuuguuggcgagauuuuu

SEQ ID NO: 93 - RNA, 20 nt, Artificial sequence
  Synthetic construct
uaauuucuacucuuguagau

SEQ ID NO: 94 - protein, 1391 aa, Artificial sequence
  Synthetic construct
GIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 95 - protein, 1398 aa, Artificial sequence
  Synthetic construct
PKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 96 - protein, 1422 aa, Artificial sequence
  Synthetic construct
DYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 97 - protein, 1076 aa, Artificial sequence
  Synthetic construct
GIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 98 - protein, 1084 aa, Artificial sequence
  Synthetic construct
APKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 99 - protein, 1113 aa, Artificial sequence
  Synthetic construct
APKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYAYPYDVPDYAYPYDVPDYA

SEQ ID NO: 100 - DNA, 142 bp, Artificial sequence
  Synthetic construct
aggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 101 - DNA, 136 bp, Artificial sequence
  Synthetic construct
cggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 102 - DNA, 178 bp, Artificial sequence
  Synthetic construct
cttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 103 - DNA, 58 bp, Artificial sequence
  Synthetic construct
acctggccaacatggtgaaacccagtatctactaaaaaatacaaaaaaaaaaaaaaaa

SEQ ID NO: 104 - DNA, 76 bp, Artificial sequence
  Synthetic construct
gtccggagttcaagactaacctggccaacatggtgaaacccagtatctactaaaaaatacaaaaaaaaaaaaaaaa

SEQ ID NO: 105 - DNA, 81 bp, Artificial sequence
  Synthetic construct
ctgaggtccggagttcaagactaacctggccaacatggtgaaacccagtatctactaaaaaatacaaaaaaaaaaaaaaaa

SEQ ID NO: 106 - DNA, 22 bp, Artificial sequence
  Synthetic construct
aataaagaaaagttagccgggc

SEQ ID NO: 107 - DNA, 86 bp, Artificial sequence
  Synthetic construct
aataaagaaaagttagccgggcgtggtgtcgcgcgcctgtaatcccagctactccagaggctgcggcaggagaatcgcttgagccc

SEQ ID NO: 108 - DNA, 49 bp, Artificial sequence
  Synthetic construct
aataaagaaaagttagccgggcgtggtgtcgcgcgcctgtaatcccagc

SEQ ID NO: 109 - DNA, 114 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaaga

SEQ ID NO: 110 - DNA, 20 bp, Artificial sequence
  Synthetic construct
aataaagaaaagttagccgg

SEQ ID NO: 111 - DNA, 126 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgca

SEQ ID NO: 112 - DNA, 519 bp, Artificial sequence
  Synthetic construct
acgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 113 - DNA, 625 bp, Artificial sequence
  Synthetic construct
acctggtgtgaggattaaatgggaataacatagataaagtcttcagaacttcaaattagttcccctttcttcctttggggggtacaaagaaatatctgacccagttacgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 114 - DNA, 507 bp, Artificial sequence
  Synthetic construct
gaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 115 - DNA, 455 bp, Artificial sequence
  Synthetic construct
ggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 116 - DNA, 456 bp, Artificial sequence
  Synthetic construct
gggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 117 - DNA, 493 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctccc

SEQ ID NO: 118 - DNA, 578 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaac

SEQ ID NO: 119 - DNA, 597 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacc

SEQ ID NO: 120 - DNA, 403 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctct

SEQ ID NO: 121 - DNA, 159 bp, Artificial sequence
  Synthetic construct
aaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 122 - DNA, 93 bp, Artificial sequence
  Synthetic construct
aaggtggatcacctgaggtccggagttcaagactaacctggccaacatggtgaaacccagtatctactaaaaaatacaaaaaaaaaaaaaaaa

SEQ ID NO: 123 - DNA, 30 bp, Artificial sequence
  Synthetic construct
ctactaaaaaatacaaaaaaaaaaaaaaaa

SEQ ID NO: 124 - DNA, 50 bp, Artificial sequence
  Synthetic construct
aataaagaaaagttagccgggcgtggtgtcgcgcgcctgtaatcccagct

SEQ ID NO: 125 - DNA, 334 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcc

SEQ ID NO: 126 - DNA, 412 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgccca

SEQ ID NO: 127 - DNA, 233 bp, Artificial sequence
  Synthetic construct
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 128 - DNA, 577 bp, Artificial sequence
  Synthetic construct
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 129 - DNA, 212 bp, Artificial sequence
  Synthetic construct
ggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 130 - DNA, 259 bp, Artificial sequence
  Synthetic construct
gtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 131 - DNA, 337 bp, Artificial sequence
  Synthetic construct
gacgcccattttgcggacctggtgtgaggattaaatgggaataacatagataaagtcttcagaacttcaaattagttcccctttcttcctttggggggtacaaagaaatatctgacccagttacgccacggctttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcc

SEQ ID NO: 132 - DNA, 335 bp, Artificial sequence
  Synthetic construct
gacgcccattttgcggacctggtgtgaggattaaatgggaataacatagataaagtcttcagaacttcaaattagttcccctttcttcctttggggggtacaaagaaatatctgacccagttacgccacggcttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaactcaggtgatccgcccacctcgccctcc

SEQ ID NO: 133 - DNA, 690 bp, Artificial sequence
  Synthetic construct
gacgcccattttgcggacctggtgtgaggattaaatgggaataacatagataaagtcttcagaacttcaaattagttcccctttcttcctttggggggtacaaagaaatatctgacccagttacgccacggcttttgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaattacaggcgtgagccaccgcgtctggccatcagcagagtttttaatttaggagaatgacaagaggtggtacagttttttagatggtacctggtggctgttaagggctattgactgacaaacacacccaacttggcgctgccgcccaggaggtggacactgggtttctggatagatggttagcaacctctgtcaccagctgggcctctttttttctatactgaattaatcacatttgtttaacctgtctgttccatagttcccttgcacatcttgggtatttgaggagttgggtgggtggcagtggcaactggggccaccatcctgtttaattattttaa

SEQ ID NO: 134 - DNA, 679 bp, Artificial sequence
  Synthetic construct
  misc_feature (2)..(2): n is a, c, g, or t
  misc_feature (5)..(6): n is a, c, g, or t
  misc_feature (8)..(8): n is a, c, g, or t
  misc_feature (80)..(80): n is a, c, g, or t
  misc_feature (504)..(504): n is a, c, g, or t
  misc_feature (519)..(519): n is a, c, g, or t
  misc_feature (533)..(533): n is a, c, g, or t
  misc_feature (543)..(543): n is a, c, g, or t
  misc_feature (548)..(548): n is a, c, g, or t
  misc_feature (597)..(598): n is a, c, g, or t
  misc_feature (624)..(624): n is a, c, g, or t
  misc_feature (635)..(635): n is a, c, g, or t
  misc_feature (641)..(641): n is a, c, g, or t
  misc_feature (644)..(644): n is a, c, g, or t
  misc_feature (648)..(648): n is a, c, g, or t
  misc_feature (663)..(663): n is a, c, g, or t
  misc_feature (666)..(666): n is a, c, g, or t
  misc_feature (669)..(669): n is a, c, g, or t
tnaanntnaacgaatcgaccgattgttaggtaatcgtcacctccacaaagagcgactcgctgtatcgctcgagggatccnaattcaggaggtaaaaaccatgatccacacgtgttatttggcccacattgtgtttgaagaaactttgggattggttgccagtgcttaaaagttaggacttagaaaatggatttcctggcaggacggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgngcttccacccctgcntgtgtggacagcangggttgtcancaganttgtgttttgttttgtttttttgagacagagtttccctcttgttgcccnncctggagcgcagtggctcaatctcanctcacagcaanatctgnatntggntttaatgattctccngcntanttcttccgtt

SEQ ID NO: 135 - DNA, 335 bp, Artificial sequence
  Synthetic construct
gactcgagaattctgacgtcattattatcagccacacgtgttatttggcccacattgtgtttgaagaaactttgggattggttgccagtgcttaaaagttaggacttagaaaatggatttcctggcaggacgtgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcc

SEQ ID NO: 136 - DNA, 336 bp, Artificial sequence
  Synthetic construct
gactcgagaattctgacgtcattattatcagccacacgtgttatttggcccacattgtgtttgaagaaactttgggattggttgccagtgcttaaaagttaggacttagaaaatggatttcctggcaggacgctgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcc

SEQ ID NO: 137 - DNA, 388 bp, Artificial sequence
  Synthetic construct
gacgcccattttgcggacctggtgtgaggattaaatgggaataacatagataaagtcttcagaacttcaaattagttcccctttcttcctttggggggtacaaagaaatatctgacccagttacgccacggcttgaaaggaggaaacccaaagaatggctgtggggatgaggaagattcctcaagtgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcc

SEQ ID NO: 138 - DNA, 247 bp, Artificial sequence
  Synthetic construct
gtgaggattaaatgggaataacatagataaagtcttcagaacttcaaattagttcccctttcttcctttggggggtacaaagaaatatctgacccagttacgccacggcttgctcaggtgatccgcccacctcgccctcccaaagtgctggaattacaggcgtgagccaccgcgtctggccatcagcagagtttttaatttaggagaatgacaagaggtggtacagttttttagatggtacctggtg

SEQ ID NO: 139 - DNA, 332 bp, Artificial sequence
  Synthetic construct
atagataaagtcttcagaacttcaaattagttcccctttcttcctttggggggtacaaagaaatatctgacccagttacgccacggcttgaaaggaggaaacccaaagaatggctgtggggatgaggaagattcctcaactcaggtgatccgcccacctcgccctcccaaagtgctggaattacaggcgtgagccaccgcgtctggccatcagcagagtttttaatttaggagaatgacaagaggtggtacagttttttagatggtacctggtggctgttaagggctattgactgacaaacacacccaacttggcgctgccgcccaggaggt

SEQ ID NO: 140 - DNA, 349 bp, Artificial sequence
  Synthetic construct
gttgctctccggagtttgtactttaggcttgaacttcccacacgtgttatttggcccacattgtgtttgaagaaactttgggattggttgccagtgcttaaaagttaggacttagaaaatggatttcctggctgttgcccaggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaat

SEQ ID NO: 141 - DNA, 428 bp, Artificial sequence
  Synthetic construct
ctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaat

SEQ ID NO: 142 - DNA, 389 bp, Artificial sequence
  Synthetic construct
gttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggggctggagtgcagtggctcagtctcagctcactgcaacctctgcctcctgggttcaagtgattctcctgcctcagcctcccgagtagctgggattatcggctaattttgtatttttagtagagacagatttctccatgttggtcaggctggtctcgaactcccaacctcaggtgatccgcccacctcgccctcccaaagtgctggaat

SEQ ID NO: 143 - DNA, 20 bp, Artificial sequence
  Synthetic construct
aagaatggctgtggggatga

SEQ ID NO: 144 - DNA, 22 bp, Artificial sequence
  Synthetic construct
ctttcatctcccctaatacatg

SEQ ID NO: 145 - DNA, 22 bp, Artificial sequence
  Synthetic construct
gtggcctgcctctttcatctcc

SEQ ID NO: 146 - DNA, 22 bp, Artificial sequence
  Synthetic construct
catatttgtgttgctctccgga

SEQ ID NO: 147 - DNA, 22 bp, Artificial sequence
  Synthetic construct
tcttcaaacacaatgtgggcca

SEQ ID NO: 148 - DNA, 22 bp, Artificial sequence
  Synthetic construct
ggcaaccaatcccaaagtttct

SEQ ID NO: 149 - DNA, 22 bp, Artificial sequence
  Synthetic construct
tccacacaggcaggggtggaag

SEQ ID NO: 150 - DNA, 22 bp, Artificial sequence
  Synthetic construct
gaggagatctaaggaccatcat

SEQ ID NO: 151 - DNA, 22 bp, Artificial sequence
  Synthetic construct
gcagacatttattacttggctt

SEQ ID NO: 152 - DNA, 22 bp, Artificial sequence
  Synthetic construct
gcccaatacgtggcagctcaga

SEQ ID NO: 153 - DNA, 22 bp, Artificial sequence
  Synthetic construct
aactctgctgacaacccatgct

SEQ ID NO: 154 - RNA, 74 nt, Campilobacter jejuni
agucccugaaaagggacuaaaauaaagaguuugcgggacucugcgggguuacaauccccuaaaaccgcuuuuuu

SEQ ID NO: 155 - protein, 987 aa, Artificial sequence
  Synthetic construct
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGP

SEQ ID NO: 156 - protein, 994 aa, Artificial sequence
  Synthetic construct
MARILAFDIGISSIGWAFSENDELKDCGVRIFTKVENPKTGESLALPRRLARSARKRLARVILHIAKRRGYDDIKNSDDKEKGAILKAIKQNEEKLANYQSVGEYLYKEYFQKFKENSKEVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGPPKKKRKV

SEQ ID NO: 157 - protein, 994 aa, Artificial sequence; identical to SEQ ID NO: 156
  Synthetic construct

SEQ ID NO: 158 - DNA, 321 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 159 - DNA, 310 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 160 - DNA, 264 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 161 - DNA, 220 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 162 - DNA, 201 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaa

SEQ ID NO: 163 - DNA, 323 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 164 - DNA, 241 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 165 - DNA, 286 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 166 - DNA, 302 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 167 - DNA, 346 bp, Artificial sequence
  Synthetic construct
aataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 168 - DNA, 100 bp, Artificial sequence
  Synthetic construct
aattcatatttgcatgtcgctatgtgttctgggaaatcaccataaacgtgaaatgtctttggatttgggaatcttataagttctgtatgagaccacggta

SEQ ID NO: 169 - DNA, 799 bp, Artificial sequence
  Synthetic construct
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 170 - DNA, 379 bp, Artificial sequence
  Synthetic construct
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtctagctttatttgtgaaatttgtgatgctattgctttatttgtaaccattataagctgcaataaacaagttaacaacaacaattgcattcattttatgtttcaggttcagggggagatgtgggaggtttttt

SEQ ID NO: 171 - DNA, 644 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 172 - DNA, 562 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 173 - DNA, 607 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 174 - DNA, 623 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 175 - DNA, 667 bp, Artificial sequence
  Synthetic construct
gtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 176 - DNA, 633 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 177 - DNA, 551 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 178 - DNA, 596 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 179 - DNA, 612 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 180 - DNA, 656 bp, Artificial sequence
  Synthetic construct
gatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 181 - DNA, 587 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 182 - DNA, 505 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 183 - DNA, 550 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 184 - DNA, 566 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 185 - DNA, 610 bp, Artificial sequence
  Synthetic construct
ggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 186 - DNA, 543 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 187 - DNA, 461 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 188 - DNA, 506 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 189 - DNA, 522 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 190 - DNA, 566 bp, Artificial sequence
  Synthetic construct
cccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 191 - DNA, 523 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggctt

SEQ ID NO: 192 - DNA, 442 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatg

SEQ ID NO: 193 - DNA, 487 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttgg

SEQ ID NO: 194 - DNA, 503 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatct

SEQ ID NO: 195 - DNA, 547 bp, Artificial sequence
  Synthetic construct
aactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtccttagatctcctctaggaaagcagacatttattacttggcttctgtgcactatctgagctgccacgtattgggcttccacccctgcctgtgtggacagc

SEQ ID NO: 196 - DNA, 592 bp, Artificial sequence
  Synthetic construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 197 - RNA, 22 nt, Artificial sequence
  Synthetic construct
cuuucaucuccccuaauacaug

SEQ ID NO: 198 - RNA, 22 nt, Artificial sequence
  Synthetic construct
guggccugccucuuucaucucc

SEQ ID NO: 199 - RNA, 22 nt, Artificial sequence
  Synthetic construct
cauauuuguguugcucuccgga

SEQ ID NO: 200 - RNA, 22 nt, Artificial sequence
  Synthetic construct
ucuucaaacacaaugugggcca

SEQ ID NO: 201 - RNA, 22 nt, Artificial sequence
  Synthetic construct
ggcaaccaaucccaaaguuucu

SEQ ID NO: 202 - RNA, 22 nt, Artificial sequence
  Synthetic construct
uccacacaggcagggguggaag

SEQ ID NO: 203 - RNA, 22 nt, Artificial sequence
  Synthetic construct
gaggagaucuaaggaccaucau

SEQ ID NO: 204 - RNA, 22 nt, Artificial sequence
  Synthetic construct
gcagacauuuauuacuuggcuu

SEQ ID NO: 205 - RNA, 22 nt, Artificial sequence
  Synthetic construct
gcccaauacguggcagcucaga

SEQ ID NO: 206 - RNA, 22 nt, Artificial sequence
  Synthetic construct
aacucugcugacaacccaugcu

SEQ ID NO: 207 - DNA, 140 bp, Homo sapiens
gtgttatttggcccacattgtgtttgaagaaactttgggattggttgccagtgcttaaaagttaggacttagaaaatggatttcctggcaggacgcggtggctcatgcccataatctcagcactttgggaggcctaggaa

SEQ ID NO: 208 - DNA, 191 bp, Homo sapiens
gtgtggccatgatggtccttagatctcctctaggaaagcagacatttattacttggcttctgtgcactatctgagctgccacgtattgggcttccacccctgcctgtgtggacagcatgggttgtcagcagagttgtgttttgttttgtttttttgagacagagtttccctcttgttgcccaggctggagt

SEQ ID NO: 209 - DNA, 433 bp, Homo sapiens
cagttacgccacggcttgaaaggaggaaacccaaagaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaa

SEQ ID NO: 210 - DNA, 84 bp, Homo sapiens
gtgtggacagcatgggttgtcagcagagttgtgttttgttttgtttttttgagacagagtttccctcttgttgcccaggctgga

SEQ ID NO: 211 - DNA, 20 bp, Artificial Sequence
  Synthetic construct
tgtcgctatgtgttctggga

SEQ ID NO: 212 - DNA, 25 bp, Artificial Sequence
  Synthetic construct
ttacgccacggcttgaaaggaggaa

SEQ ID NO: 213 - DNA, 24 bp, Artificial Sequence
  Synthetic construct
agtttcctcttgttgcccaggctg

SEQ ID NO: 214 - DNA, 14 bp, Artificial Sequence
  Synthetic construct
ttacgccacggctt

SEQ ID NO: 215 - DNA, 15 bp, Artificial Sequence
  Synthetic construct
ttgttgcccaggctg

SEQ ID NO: 216 - DNA, 13 bp, Artificial Sequence
  Synthetic construct
ttacgccacggct

SEQ ID NO: 217 - DNA, 14 bp, Artificial Sequence
  Synthetic construct
tgttgcccaggctg

SEQ ID NO: 218 - DNA, 25 bp, Artificial Sequence
  Synthetic construct
ttcctggcaggacgcggtggctcat

SEQ ID NO: 219 - DNA, 25 bp, Artificial Sequence
  Synthetic construct
gaaaagttagccgggcgtggtgtcg

SEQ ID NO: 220 - DNA, 14 bp, Artificial Sequence
  Synthetic construct
ttcctggcaggacg

SEQ ID NO: 221 - DNA, 11 bp, Artificial Sequence
  Synthetic construct
gcgtggtgtcg

SEQ ID NO: 222 - DNA, 25 bp, Artificial Sequence
  Synthetic construct
agattcctcaaggggaggacatggt

SEQ ID NO: 223 - DNA, 12 bp, Artificial Sequence
  Synthetic construct
agattcctcaag